(19) United States
(12) Patent Application Publication
(10) Pub. No.: US 2023/0381176 A1
(43) Pub. Date: Nov. 30, 2023

(54) A PHARMACEUTICAL COMPOSITION AND USE THEREOF FOR TREATMENT OF CANCER

(71) Applicants: Ascentage Pharma (Suzhou) Co., Ltd., Suzhou (CN); Ascentage Pharma Group Corp Limited, Hong Kong (CN)

(72) Inventors: Yifan Zhai, Suzhou (CN); Dajun Yang, Suzhou (CN); Douglas Fang, Suzhou (CN); Ran Tao, Suzhou (CN); Guangfeng Wang, Suzhou (CN)

(21) Appl. No.: 18/028,317

(22) PCT Filed: Sep. 24, 2021
(86) PCT No.: PCT/CN2021/120260

§ 371 Date: Mar. 24, 2023

(30) Foreign Application Priority Data
Sep. 25, 2020 (WO) PCT/CN2020/117895

(51) Int. Cl. A61K 31/506; A61K 31/519; A61K 39/395; A61K 31/517; A61K 31/4184; A61K 31/407; A61K 31/4045; A61K 31/502; A61K 31/47; A61K 31/496; A61P 35/00

(52) U.S. Cl. CPC A61K 31/506; A61K 31/519; A61K 39/3955; A61K 31/517; A61K 31/4184; A61K 31/407; A61K 31/4045; A61K 31/502; A61K 31/47; A61K 31/496; A61P 35/00

(57) ABSTRACT

Provided herein in the biomedical field are a pharmaceutical composition, use thereof for treating or suppressing a cancer, reducing its severity, lowering its risk or inhibiting its metastasis in an individual, and a method for treating or suppressing a cancer, reducing its severity, lowering its risk or inhibiting its metastasis in an individual. The method comprises administering to the individual a therapeutically effective amount of an ALK inhibitor, and a therapeutically effective amount of one or more other anticancer reagents. It also relates to a kit comprising an ALK inhibitor, and one or more other anticancer reagents.

### TECHNICAL FIELD

The present invention pertains to the biomedical field, and particularly relates to a method for treating or suppressing a cancer, reducing its severity, lowering its risk or inhibiting its metastasis in an individual. The method comprises administering to the individual a therapeutically effective amount of an ALK inhibitor, and a therapeutically effective amount of one or more other anticancer reagents. The invention also relates to a pharmaceutical composition or kit comprising an ALK inhibitor, and one or more other anticancer reagents.

### BACKGROUND OF THE INVENTION

Proliferative diseases represent a serious threat to modern society. Cancerous growths pose serious challenges for modern medicine due to their unique characteristics, including uncontrollable cell proliferation, an ability to invade local and even remote tissues, lack of differentiation, lack of detectable symptoms and lack of effective therapy and prevention. Worldwide, more than 10 million people are diagnosed with cancer every year, and cancer causes six million deaths every year or 12% of the deaths worldwide.

Small molecule ALK inhibitors can be designed to target ALK mutations, so as to treat related diseases and conditions including cancer. ALK mutations targeted drugs include: crizotinib as first-generation, ceritinib, alectinib and brigatinib as second-generation, lorlatinib as third-generation, and the like. However, the targeted drugs usually exhibit resistance about 1 year after administration. Thus, overcoming the drug resistance of the targeted drugs as well as other anticancer reagents and improving the efficacy are some of the main objectives in drug research and development.

### SUMMARY OF THE INVENTION

In one aspect, the invention provides a method for treating or suppressing a cancer, reducing its severity, lowering its risk or inhibiting its metastasis in an individual, said method comprising administering to the individual a therapeutically effective amount of an ALK inhibitor, and a therapeutically effective amount of one or more anticancer reagents selected from a CDK4/6 inhibitor, an Mek inhibitor, a BRAF inhibitor, a chemotherapeutic agent, an MDM2 inhibitor, an HDAC inhibitor, a PD-1 inhibitor, a PARP inhibitor, a VEGF inhibitor and a BCR-ABL inhibitor.

In another aspect, the invention provides a use of an ALK inhibitor in manufacture of a medicament in combination with one or more anticancer reagents selected from a CDK4/6 inhibitor, an Mek inhibitor, a BRAF inhibitor, a chemotherapeutic agent, an MDM2 inhibitor, an HDAC inhibitor, a PD-1 inhibitor, a PARP inhibitor, a VEGF inhibitor and a BCR-ABL inhibitor for treating or suppressing a cancer, reducing its severity, lowering its risk or inhibiting its metastasis in an individual.

In another aspect, the invention provides a use of a pharmaceutical composition for treating or suppressing a cancer, reducing its severity, lowering its risk or inhibiting its metastasis in an individual, said pharmaceutical composition comprising an ALK inhibitor as well as one or more anticancer reagents selected from a CDK4/6 inhibitor, an Mek inhibitor, a BRAF inhibitor, a chemotherapeutic agent, an MDM2 inhibitor, an HDAC inhibitor, a PD-1 inhibitor, a PARP inhibitor, a VEGF inhibitor and a BCR-ABL inhibitor.

In another aspect, the invention provides an ALK inhibitor which is used in combination with one or more anticancer reagents selected from a CDK4/6 inhibitor, an Mek inhibitor, a BRAF inhibitor, a chemotherapeutic agent, an MDM2 inhibitor, an HDAC inhibitor, a PD-1 inhibitor, a PARP inhibitor, a VEGF inhibitor and a BCR-ABL inhibitor for treating or suppressing a cancer, reducing its severity, lowering its risk or inhibiting its metastasis in an individual.

In another aspect, the invention provides a pharmaceutical composition comprising an ALK inhibitor, and one or more anticancer reagents selected from a CDK4/6 inhibitor, an Mek inhibitor, a BRAF inhibitor, a chemotherapeutic agent, an MDM2 inhibitor, an HDAC inhibitor, a PD-1 inhibitor, a PARP inhibitor, a VEGF inhibitor and a BCR-ABL inhibitor, as well as a pharmaceutically acceptable carrier.

In another aspect, the invention provides a kit, comprising:
(a) a first component in a first container, the first component comprising an ALK inhibitor, and optionally a pharmaceutically acceptable carrier;
(b) a second component in a second container, the second component comprising one or more anticancer reagents selected from a CDK4/6 inhibitor, an Mek inhibitor, a chemotherapeutic agent, an MDM2 inhibitor, an HDAC inhibitor, a PD-1 inhibitor, a PARP inhibitor, a VEGF inhibitor and a BCR-ABL inhibitor, and optionally a pharmaceutically acceptable carrier; and
(c) an optional specification.

### BRIEF DESCRIPTION OF THE DRAWINGS

FIG. 1 shows that a combination of Compound 5 with Compound 34 with the following structure enhances inhibition effect on the proliferation of NCI-H2228 tumor cells.

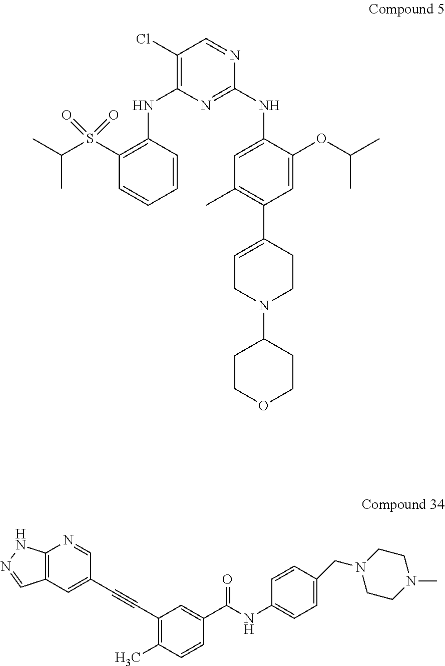 [SMILES: Cc1cc(Nc2ncc(Cl)c(Nc3ccccc3S(=O)(=O)C(C)C)n2)c(OC(C)C)cc1C1=CCN(C2CCOCC2)CC1 ; Cc1ccc(C(=O)Nc2ccc(CN3CCN(C)CC3)cc2)cc1C#Cc1cnc2[nH]ncc2c1]

FIG. 2 shows that a combination of Compound 5 and Compound 33 with the following structure including any tautomer forms demonstrates enhanced cell viability inhibition in Uveal melanoma MP41 cells.

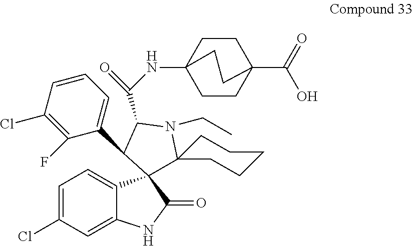 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

FIG. 3 shows synergistic anti-tumor effect of Compound 5 in combination with palbociclib in Mesothelioma MT16036 PDX model and corresponding body weight change.

FIG. 4 shows synergistic anti-tumor effect of Compound 5 in combination with ribociclib in Neuroblastoma SH-SYSY (ALK F1174L) model and corresponding body weight change.

FIG. 5 shows synergistic anti-tumor effect of Compound 5 in combination with trametinib/selumetinib in A549 NSCLC model and corresponding body weight change.

FIG. 6 shows synergistic anti-tumor effect of Compound 5 in combination with carboplatin in PTK2 high NSCLC PDX model and corresponding body weight change.

FIG. 7 shows Synergistic anti-tumor effect of Compound 5 in combination with carboplatin in ovarian PA-1 model and corresponding body weight change.

FIG. 8 shows synergistic anti-tumor effect of Compound 5 in combination with paclitaxel or Compound 5 in combination with carboplatin in PTK2 high ovarian PDX OV2423 model, as well as corresponding body weight change.

FIG. 9 shows synergistic anti-tumor effect of Compound 5 in combination with paclitaxel or Compound 5 in combination with paclitaxel plus carboplatin in ovarian PDX OV1658 model, as well as corresponding body weight change.

FIG. 10 shows synergistic anti-tumor effect of Compound 5 in combination with paclitaxel or Compound 5 in combination with paclitaxel plus carboplatin in PTK2 high ovarian PDX OV1385 model, as well as corresponding body weight change.

FIG. 11 shows synergistic anti-tumor effect of Compound 5 in combination with paclitaxel in PTK2 high ovarian PDX model OV2018 and corresponding body weight change.

FIG. 12 shows synergistic anti-tumor effect of Compound 5 in combination with paclitaxel or Compound 5 in combination with paclitaxel plus carboplatin in OVCAR3 ovarian tumor model, as well as corresponding body weight change.

FIG. 13 shows that Compound 5 may overcome resistance to chemotherapeutic agents in OVCAR3 ovarian tumor model.

FIG. 14 shows synergistic anti-tumor effect of Compound 5 in combination with Compound 33 in A549 NSCLC xenograft tumor model, as well as corresponding body weight change.

FIG. 15 shows synergistic anti-tumor effect of Compound 5 in combination with Compound 33 in Neuroblastoma SH-SYSY xenograft tumor model, as well as corresponding body weight change.

FIG. 16 shows synergistic anti-tumor effect of Compound 5 in combination with panobinostat in A549 NSCLC xenograft tumor model, as well as corresponding body weight change.

FIG. 17 shows synergistic anti-tumor effect of Compound 5 in combination with anti-PD-1 antibody in syngeneic tumor model of colon CT26, as well as corresponding body weight change.

FIG. 18 shows synergistic anti-tumor effect of Compound 5 in combination with Olaparib in NSCLC LU-01-0751 PDX xenograft tumor model, as well as corresponding body weight change.

FIG. 19 shows synergistic anti-tumor effect of Compound 5 in combination with Lenvatinib in PTK2-high liver PDX model of LI-03-1140, as well as corresponding body weight change.

FIG. 20 shows synergistic anti-tumor effect of Compound 5 in combination with BRAFi+MEKi in TP53wt, BRAFv600E, NRASwt, PTENwt, CDKN2Amut C32 cutaneous melanoma model as well as corresponding body weight change.

FIG. 21 shows synergistic anti-tumor effect of Compound 5 in combination with Palbociclib/Trametinib/TMZ in U-87-MG subcutaneous glioblastoma, as well as corresponding body weight change.

### DETAILED DESCRIPTION OF THE INVENTION

Unless otherwise defined below, all technical and scientific terms used herein have the same meanings as commonly understood by one of ordinary skill in the art. References to techniques used herein are intended to refer to techniques that are generally understood in the art, including those obvious changes or equivalent replacements of the techniques for those skilled in the art. While it is believed that the following terms are well understood by those skilled in the art, the following definitions are set forth to better explain the invention.

As used herein, the terms “including”, “comprising”, “having”, “containing” or “comprising”, and other variants thereof, are inclusive or open, and do not exclude other unlisted elements or method steps.

As used herein, “ALK” refers to anaplastic lymphoma kinase, and “ALK inhibitor” refers to an agent having an inhibitory effect on ALK. In some embodiments, the ALK inhibitor also has an inhibitory effect on one or more other targets (e.g., FAK (focal adhesion kinase) and/or ROS1 (a tyrosine protein kinase encoded by ROS1 proto-oncogene in human)).

As used herein, “CDK4/6 inhibitor” refers to an agent that selectively and efficiently inhibits cyclin-dependent kinase 4 or cyclin-dependent kinase 6 (CDK4/6).

As used herein, “Mek inhibitor” refers to an agent that inhibits mitogen-activated protein kinase (MEK), and MEK is a major protein in the RAS/RAF/MEK pathway, which signals toward cell proliferation and survival, and frequently activated in tumors that have mutations in the RAS or RAF oncogenes or in growth receptor tyrosine kinases.

As used herein, BRAF inhibitor refers to an agent that inhibits BRAF, such as Dabrafenib, Sorafenib, Regorafenib, Pazopanib, Vemurafenib etc.

As used herein, “chemotherapeutic agent” refers to chemotherapeutic drugs that can kill tumor cells, and these drugs can act on different stages of tumor cell growth and reproduction, thereby inhibit or kill tumor cells.

As used herein, “MDM2 inhibitor” refers to Murine Double Minute 2 (MDM2) inhibitor which interferes with the binding of MDM2 oncoprotein to the tumor suppressor p53 protein, and serves as pharmacological p53 activators.

As used herein, “HDAC inhibitor” refers to an agent that inhibits histone deacetylases (HDAC), and has been described to cause growth arrest with subsequent differentiation or apoptosis of tumor cells, whereas normal cells are not affected.

As used herein, “PD-1 inhibitor” refers to an agent that targets the programmed death 1 (PD-1) signaling pathway, and can be anti-PD-1 antibody. The anti-PD-1 antibody can be monoclonal antibody or bispecific antibody, it may be full length antibody or antibody fragment, as long as it can block the binding between PD-1 and PD-L1.

As used herein, “PARP inhibitor” refers to an agent that inhibits poly ADP ribose polymerase (PARP).

As used herein, “VEGF inhibitor” refers to an agent that targets vascular endothelial growth factor (VEGF) signaling pathway, wherein VEGF is a major regulatory factor of angiogenesis, and in most human tumors is involved in tumor growth and metastasis.

As used herein, “BCR-ABL inhibitor” refers to an agent that targets the fusion gene of abelson murine leukemia (Abl) and breakpoint cluster region (Bcr).

The term “alkyl” as used herein, alone or as part of another group, refers to an unsubstituted straight or branched aliphatic hydrocarbon containing from 1 to 12 carbon atoms (ie, C₁₋₁₂ alkyl) or an indicated number of carbon atoms, for example, C₁ alkyl such as methyl, C₂ alkyl such as ethyl, C₃ alkyl such as n-propyl or isopropyl, C₁₋₃ alkyl such as methyl, ethyl, n-propyl or isopropyl, or the like. In one embodiment, the alkyl is C₁₋₄ alkyl. Non-limiting examples of C₁₋₁₂ alkyl include methyl, ethyl, n-propyl, isopropyl, n-butyl, sec-butyl, tert-butyl, isobutyl, 3-pentyl, hexyl, heptyl, octyl, nonyl and decyl. Examples of C₁₋₄ alkyl include methyl, ethyl, n-propyl, isopropyl, n-butyl, sec-butyl, tert-butyl, and isobutyl.

The term “cycloalkyl” as used herein, alone or as part of another group, refers to a saturated or partially unsaturated (containing one or two double bonds) cyclic aliphatic hydrocarbon, which comprises 1 or 2 rings having 3 to 12 carbon atoms or an indicated number of carbon atoms (i.e., C₃₋₁₂ cycloalkyl).

In one embodiment, the cycloalkyl has two rings. In one embodiment, the cycloalkyl has one ring. In another embodiment, the cycloalkyl group is selected from the group consisting of C₃₋₈ cycloalkyl groups. In another embodiment, the cycloalkyl group is selected from the group consisting of C₃₋₆ cycloalkyl groups. Non-limiting examples of cycloalkyl include cyclopropyl, cyclobutyl, cyclopentyl, cyclohexyl, cycloheptyl, cyclooctyl, norbornyl, decahydronaphthyl, adamantyl, cyclohexenyl, and cyclopentenyl.

The term “heterocycle” or “heterocyclyl” as used herein, alone or as part of another group, refers to a saturated or partially unsaturated (e.g., comprising one or two double bonds) cyclic group, which comprises 1, 2 or 3 rings having 3 to 14 ring members (i.e., 3- to 14-membered heterocyclyl), wherein at least one carbon atom of one of the rings is replaced by a heteroatom. Each heteroatom is independently selected from the group consisting of atoms of oxygen, sulfur (including sulfoxide and sulfone) and/or nitrogen (which may be oxidized or quaternized). The term “heterocyclyl” is intended to include a group wherein —CH₂— in the ring is replaced by —C(═O)—, for example, cyclic ureido (such as 2-imidazolidinone) and cyclic amido (such as β-lactam, γ-lactam, δ-lactam, ε-lactam) and piperazin-2-one. In one embodiment, the heterocyclyl is a 3- to 8-membered cyclic group comprising 1 ring and 1 or 2 oxygen and/or nitrogen atoms. In one embodiment, the heterocyclyl is a 4-, 5- or 6-membered cyclic group comprising 1 ring and 1 or 2 oxygen and/or nitrogen atoms. In one embodiment, the heterocyclyl is a 4- or 6-membered cyclic group comprising 1 ring and 1 or 2 oxygen and/or nitrogen atoms. The heterocyclyl can be attached to the remainder of molecule via any available carbon or nitrogen atom. Non-limiting examples of the heterocyclyl include dioxanyl, tetrahydropyranyl, 2-oxopyrrolidin-3-yl, piperazin-2-one, piperazin-2,6-dione, 2-imidazolidinone, piperidinyl, morpholinyl, piperazinyl, pyrrolidinyl and dihydroindolyl.

As used herein, the term “enantiomeric excess” or “ee” refers to a measure of how much one enantiomer is present relative to another enantiomer. For a mixture of R and S enantiomers, the enantiomeric excess in form of percentage is defined as |R−S|*100, wherein R and S respectively represents mole or weight parts thereof in the mixture, and R+S=1. After knowing the optical rotation of chiral substance, the enantiomeric excess in form of percentage is defined as ([α]_{obs}/[α]ₘₐₓ)*100, wherein [α]_{obs} represents the optical rotation of the mixture of enantiomers, [α]ₘₐₓ represents the optical rotation of pure enantiomer. Enantiomeric excess can be determined using a variety of analytical techniques, including NMR spectroscopy, chiral column chromatography, or optical rotation. The compound of the present invention may have an ee of about 70% or more, such as about 80% or more, 90% or more, 91% or more, 92% or more, 93% or more, 94% or more, 95% or more, 96% or more, 97% or more, 98% or more, or 99% or more.

The term “pharmaceutically acceptable salt”, as used herein, includes both acid addition salts and base addition salts of a compound.

Suitable acid addition salts are formed from acids which form non-toxic salts. Examples include acetate, adipate, aspartate, benzoate, besylate, bicarbonate/carbonate, bisulfate/sulfate, borate, camphorsulfonate, citrate, cyclohexylaminosulfonate, ethanedisulfonate, ethanesulfonate, formate, fumarate, glucoheptonate, gluconate, glucuronate, hexafluorophosphate, hibenzate, hydrochloride/chloride, hydrobromide/bromide, hydroiodide/iodide, isethionate, lactate, malate, maleate, malonate, methanesulfonate, methylsulfate, naphthylate, 2-naphthalenesulfonate, nicotinate, nitrate, orotate, oxalate, palmitate, pamoate, phosphate/hydrogen phosphate/dihydrogen phosphate, pyroglutamate, aldarate, stearate, succinate, tannate, tartrate, tosylate, trifluoroacetate and xinofoate.

Suitable base addition salts are formed from bases which form non-toxic salts. Examples include aluminum salts, arginine salts, benzathine benzylpenicillin salts, calcium salts, choline salts, diethylamine salts, diethanolamine salts, glycine salts, lysine salts, magnesium salts, meglumine salts, ethanolamine salts, potassium salts, sodium salts, tromethamine salts and zinc salts.

For a review of suitable salts, see “Handbook of Pharmaceutical Salts: Properties, Selection, and Use” by Stahl and Wermuth (Wiley-VCH, 2002). Methods for preparing the pharmaceutically acceptable salts of the compounds of the invention are known to those skilled in the art.

The term “solvate” as used herein is a substance formed by combination, physical binding and/or solvation of a compound of the invention with a solvent molecule, such as a disolvate, a monosolvate or a hemisolvate, wherein the ratio of the solvent molecule to the compound of the invention is about 2:1, about 1:1 or about 1:2, respectively. This kind of physical bonding involves ionization and covalent bonding (including hydrogen bonding) in different degrees. In some cases (e.g., when one or more solvent molecules are incorporated into crystal lattice of crystalline solid), the solvate can be isolated.

Thus, the solvate comprises both solution phase and isolatable solvates. The compounds of the invention may be in solvated forms with pharmaceutically acceptable solvents (such as water, methanol and ethanol), and the present application is intended to encompass both solvated and unsolvated forms of the compounds of the invention.

One type of solvate is a hydrate. “Hydrate” relates to a specific subset of solvates wherein the solvent molecule is water. Solvates generally function in the form of pharmacological equivalents. The preparation of solvates is known in the art, see for example, M. Caira et al, J. Pharmaceut. Sci., 93(3): 601-611 (2004), which describes the preparation of a solvate of fluconazole with ethyl acetate and water. Similar methods for the preparation of solvates, hemisolvates, hydrates and the like are described by van Tonder et al, AAPS Pharm. Sci. Tech., 5(1): Article 12 (2004) and A. L. Bingham et al, Chem. Commun. 603-604 (2001). A representative and non-limiting method for the preparation of solvate involves dissolving a compound of the invention in a desired solvent (organic solvent, water or a mixture thereof) at a temperature above 20° C. to about 25° C., and then the solution is cooled at a rate sufficient to form a crystal, and the crystal is separated by a known method such as filtration. Analytical techniques such as infrared spectroscopy can be used to confirm the presence of the solvent in the crystal of the solvate.

“Pharmaceutically acceptable carrier” in the context of the present invention refers to a diluent, adjuvant, excipient or vehicle together with which the therapeutic agent is administered, and which is suitable for contacting a tissue of human and/or other animals within the scope of reasonable medical judgment, and without excessive toxicity, irritation, allergic reactions, or other problems or complications corresponding to a reasonable benefit/risk ratio.

The pharmaceutically acceptable carriers that can be used in the pharmaceutical compositions or kits of the invention include, but are not limited to, sterile liquids such as water and oils, including those oils derived from petroleum, animals, vegetables or synthetic origins, for example, peanut oil, soybean oil, mineral oil, sesame oil, etc. Water is an exemplary carrier when the pharmaceutical composition is administered intravenously. It is also possible to use physiological saline and an aqueous solution of glucose and glycerin as a liquid carrier, particularly for injection. Suitable pharmaceutical excipients include starch, glucose, lactose, sucrose, gelatin, maltose, chalk, silica gel, sodium stearate, glyceryl monostearate, talc, sodium chloride, skimmed milk powder, glycerin, propylene glycol, water, ethanol and the like. The pharmaceutical composition may further contain a small amount of a wetting agent, an emulsifier or a pH buffering agent as needed. Oral formulations may contain standard carriers such as pharmaceutical grades of mannitol, lactose, starch, magnesium stearate, sodium saccharin, cellulose, magnesium carbonate, and the like. Examples of suitable pharmaceutically acceptable carriers are as described in Remington's Pharmaceutical Sciences (1990).

The pharmaceutical compositions and the components of the kit of the invention may act systemically and/or locally. For this purpose, they may be administered via a suitable route, for example by injection (e.g., intravenous, intraarterial, subcutaneous, intraperitoneal, intramuscular administration, including instillation) or transdermal administration; or by oral, buccal, nasal, transmucosal, topical administration, in form of ophthalmic preparation or by inhalation.

For these routes of administration, the pharmaceutical compositions and the components of the kit of the invention may be administered in a suitable dosage form.

The dosage forms include, but are not limited to, tablets, capsules, troches, hard candy, pulvis, sprays, creams, ointments, suppositories, gels, pastes, lotions, ointments, aqueous suspensions, injectable solutions, elixirs, syrups.

The term “container” as used herein refers to a container for holding a pharmaceutical component. This container can be used for preparation, storage, transportation and/or stand-alone/bulk sale, which is intended to include bottles, cans, vials, flasks, syringes, tubes (e.g., those used in cream products), or any other containers for preparation, containment, storage or distribution of a drug product.

The term “specification/instruction” as used herein refers to an insert, a tag, a label, etc., which records information about a pharmaceutical component located in the container. The information as recorded is typically determined by the regulatory agency (e.g., the United States Food and Drug Administration) that governs the area in which the product is to be sold. Preferably, the package leaflet specifically lists an indication for which the use of the pharmaceutical component is approved. The package leaflet can be made of any material from which information contained therein or thereon can be read. Preferably, the package leaflet is a printable material (e.g., paper, plastic, cardboard, foil, adhesive paper or plastic, etc.) on which the desired information can be formed (e.g., printed or applied).

The term “effective amount” as used herein refers to an amount of active ingredient that, after administration, will relieve to some extent one or more symptoms of the condition being treated.

As used herein, “individual” includes a human or a non-human animal Exemplary human individual includes a human individual (referred to as a patient) suffering from a disease (such as the disease described herein) or a normal individual. “Non-human animal” in the present invention includes all vertebrates, such as non-mammals (e.g., birds, amphibians, reptiles) and mammals, such as non-human primates, domestic animals, and/or domesticated animals (e.g., sheep, dogs, cats, cows, pigs, etc.).

As used herein, “cancer metastasis” refers to a cancer that spreads (metastasizes) from its original site to another area of the body. Almost all cancers have the potential to metastasize. Whether metastasis will occur depends on complex interactions between multiple tumor cell factors (including type of cancer, degree of maturation (differentiation) of tumor cells, location and age of cancer, and other factors that are not fully understood). There are three ways of metastasis: local expansion from a tumor to a surrounding tissue, arrival through bloodstream to a distant site, or arrival through lymphatic system to an adjacent or distant lymph node. Each cancer can have a representative diffusion route. Tumors are named according to their primary sites (for example, breast cancer that has metastasized to the brain is called metastatic breast cancer that metastasizes to the brain).

As used herein, “resistance” refers to that a cancer cell is resistant to chemotherapy. Cancer cells may acquire resistance to chemotherapy through a range of mechanisms, including mutation or overexpression of drug targets, inactivation of drugs, or elimination of drugs from cells.

### Therapeutic Methods and Uses

In one embodiment, the invention provides a pharmaceutical composition comprising an ALK inhibitor, and one or more anticancer reagents selected from a CDK4/6 inhibitor, an Mek inhibitor, a chemotherapeutic agent, an MDM2 inhibitor, an HDAC inhibitor, a PD-1 inhibitor, a PARP inhibitor, a VEGF inhibitor and a BCR-ABL inhibitor.

In a preferred embodiment, the ALK inhibitor is a compound of Formula I or a pharmaceutically acceptable salt or solvate thereof:

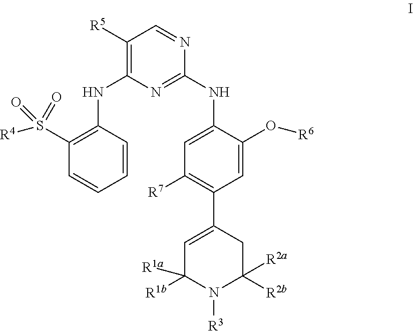

wherein:
R^{1a} and R^{1b} are independently selected from the group consisting of hydrogen, C₁₋₆ alkyl, and C₃₋₈ cycloalkyl;
R^{2a} and R^{2b} are independently selected from the group consisting of hydrogen, C₁₋₆ alkyl, and C₃₋₈ cycloalkyl;
R³ is selected from the group consisting of hydrogen, C₁₋₆ alkyl, C₃₋₆ cycloalkyl, and 4- to 8-membered heterocyclyl,
R⁴ is selected from the group consisting of C₁₋₄ alkyl and C₃₋₆ cycloalkyl;
R⁵ is halo;
R⁶ is selected from the group consisting of C₁₋₄ alkyl and C₃₋₆ cycloalkyl; and
R⁷ is selected from the group consisting of hydrogen, C₁₋₄ alkyl, and C₃₋₆ cycloalkyl,
with proviso that when R^{1a}, R^{1b}, R^{2a}, and R^{2b} are each hydrogen, then R³ is selected from the group consisting of C₃₋₆ cycloalkyl and 4- to 8-membered heterocyclyl.

In a preferred embodiment, the ALK inhibitor is a compound of Formula II or a pharmaceutically acceptable salt or solvate thereof:

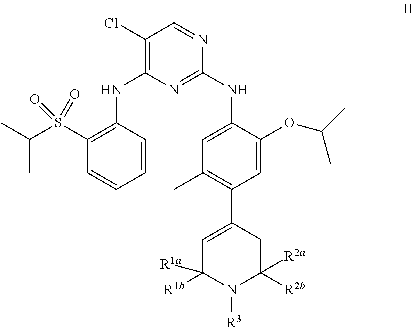

wherein:
R^{1a} and R^{1b} are independently selected from the group consisting of hydrogen, C₁₋₄ alkyl, and C₃₋₆ cycloalkyl;
R^{2a} and R^{2b} are independently selected from the group consisting of hydrogen, C₁₋₄ alkyl, and C₃₋₆ cycloalkyl; and
R³ is selected from the group consisting of hydrogen, C₁₋₄ alkyl, C₃₋₆ cycloalkyl, and 4- to 8-membered heterocyclyl.

In a preferred embodiment, the ALK inhibitor is a compound of Formula III or a pharmaceutically acceptable salt or solvate thereof:

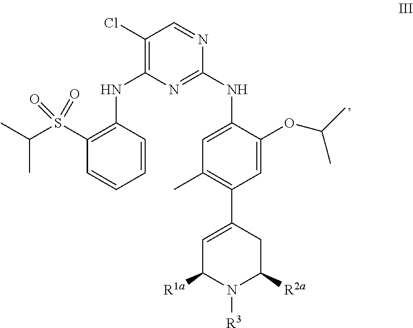

wherein:
R^{1a} and R^{2a} are each independently selected from the group consisting of C₁₋₄ alkyl, and C₃₋₆ cycloalkyl; and
the compound has an enantiomeric excess of about 90% or more. In some embodiments, the compound has an enantiomeric excess of about 91% or more, about 92% or more, about 93% or more, about 94% or more, about 95% or more, about 96% or more, about 97% or more, about 98% or more, or about 99% or more.

In a preferred embodiment, the ALK inhibitor is a compound of Formula IV or a pharmaceutically acceptable salt or solvate thereof:

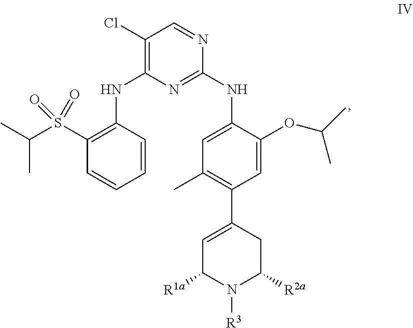

wherein:
R^{1a} and R^{2a} are each independently selected from the group consisting of C₁₋₄ alkyl, and C₃₋₆ cycloalkyl; and
the compound has an enantiomeric excess of about 90% or more.

In some embodiments, the compound has an enantiomeric excess of about 91% or more, about 92% or more, about 93% or more, about 94% or more, about 95% or more, about 96% or more, about 97% or more, about 98% or more, or about 99% or more.

In a preferred embodiment, the ALK inhibitor is a compound of Formula V or a pharmaceutically acceptable salt or solvate thereof:

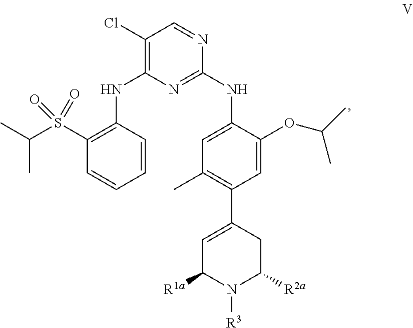

wherein:
R^{1a} and Rea are each independently selected from the group consisting of C₁₋₄ alkyl, and C₃₋₆ cycloalkyl; and
   the compound has an enantiomeric excess of about 90% or more. In some embodiments, the compound has an enantiomeric excess of about 91% or more, about 92% or more, about 93% or more, about 94% or more, about 95% or more, about 96% or more, about 97% or more, about 98% or more, or about 99% or more.

In a preferred embodiment, the ALK inhibitor is a compound of Formula VI or a pharmaceutically acceptable salt or solvate thereof:

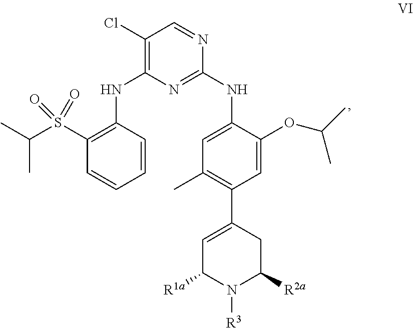

wherein:
R^{1a} and R^{2a} are each independently selected from the group consisting of C₁₋₄ alkyl, and C₃₋₆ cycloalkyl; and
the compound has an enantiomeric excess of about 90% or more. In some embodiments, the compound has an enantiomeric excess of about 91% or more, about 92% or more, about 93% or more, about 94% or more, about 95% or more, about 96% or more, about 97% or more, about 98% or more, or about 99% or more.

In a preferred embodiment, the ALK inhibitor is a compound in the following table or a pharmaceutically acceptable salt or solvate thereof:

| No. | Structure | Name |
|---|---|---|
| 1 | 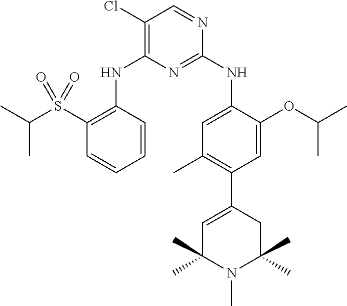 | 5-chloro-N²-(2-isopropoxy-5-methyl-4-(1,2,2,6,6- pentamethyl-1,2,3,6-tetrahydropyridin-4-yl)phenyl)- N⁴-(2-(isopropylsulfonyl)phenyl)pyrimidine-2,4- diamine; |
| 2 | 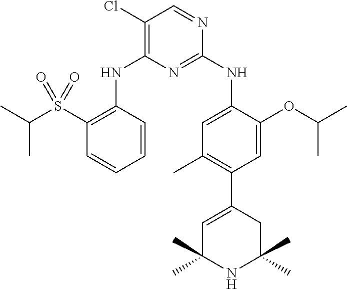 | 5-chloro-N²-(2-isopropoxy-5-methyl-4-(2,2,6,6- tetramethyl-1,2,3,6-tetrahydropyridin-4-yl)phenyl)- N⁴-(2-(isopropylsulfonyl)phenyl)pyrimidine-2,4- diamine; |
| 3 | 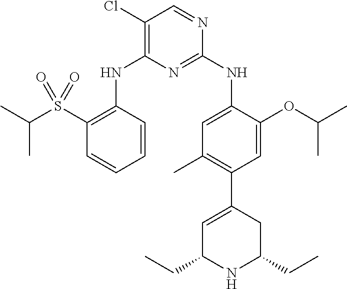 [SMILES: CC[C@H]1CC(c2cc(OC(C)C)c(Nc3ncc(Cl)c(Nc4ccccc4S(=O)(=O)C(C)C)n3)cc2C)=C[C@@H](CC)N1] | 5-chloro-N²-(4-((cis)-2,6-diethyl-1,2,3,6- tetrahydropyridin-4-yl)-2-isopropoxy-5-methylphenyl)- N⁴-(2-(isopropylsulfonyl)phenyl)pyrimidine-2,4- diamine; |
| 4 | 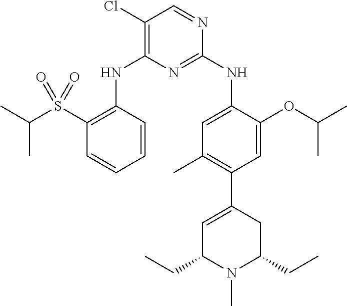 [SMILES: CC[C@H]1CC(c2cc(OC(C)C)c(Nc3ncc(Cl)c(Nc4ccccc4S(=O)(=O)C(C)C)n3)cc2C)=C[C@@H](CC)N1C] | 5-chloro-N²-(4-((cis)-2,6-diethyl-1-methyl-1,2,3,6- tetrahydropyridin-4-yl)-2-isopropoxy-5-methylphenyl)- N⁴-(2-(isopropylsulfonyl)phenyl)pyrimidine-2,4- diamine; |
| 5 | 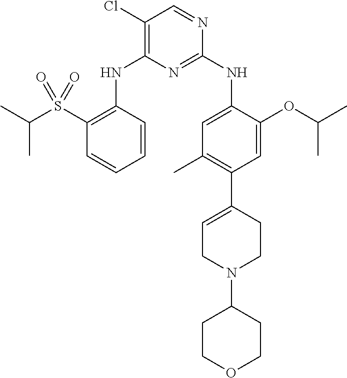 [SMILES: Cc1cc(Nc2ncc(Cl)c(Nc3ccccc3S(=O)(=O)C(C)C)n2)c(OC(C)C)cc1C1=CCN(C2CCOCC2)CC1] | 5-chloro-N²-(2-isopropoxy-5-methyl-4-(1-(tetrahydro- 2H-pyran-4-yl)-1,2,3,6-tetrahydropyridin-4-yl) phenyl)-N⁴-(2-(isopropylsulfonyl)phenyl)pyrimidine- 2,4-diamine; |
| 6 | 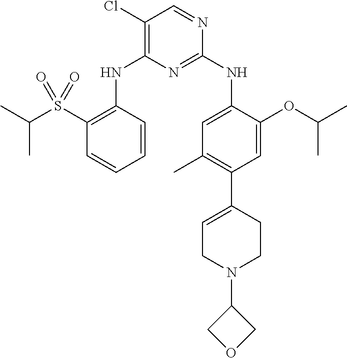 [SMILES: Cc1cc(Nc2ncc(Cl)c(Nc3ccccc3S(=O)(=O)C(C)C)n2)c(OC(C)C)cc1C1=CCN(C2COC2)CC1] | 5-chloro-N²-(2-isopropoxy-5-methyl-4-(1-(oxetan- 3-yl)-1,2,3,6-tetrahydropyridin-4-yl)phenyl)-N⁴-(2- (isopropylsulfonyl)phenyl)pyrimidine-2,4-diamine; |
| 7 | 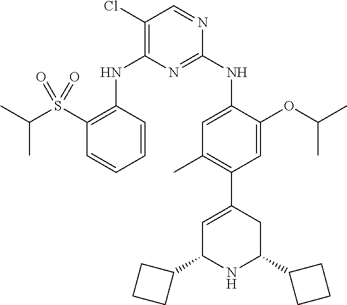 [SMILES: Cc1cc(Nc2ncc(Cl)c(Nc3ccccc3S(=O)(=O)C(C)C)n2)c(OC(C)C)cc1C1=C[C@@H](C2CCC2)N[C@@H](C2CCC2)C1] | 5-chloro-N²-(4-((cis)-2,6-dicyclobutyl-1,2,3,6- tetrahydropyridin-4-yl)-2-isopropoxy-5-methylphenyl)- N⁴-(2-(isopropylsulfonyl)phenyl)pyrimidine-2,4- diamine; |
| 8 | 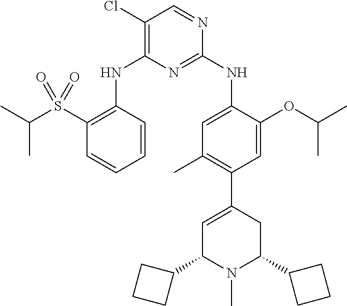 [SMILES: Cc1cc(Nc2ncc(Cl)c(Nc3ccccc3S(=O)(=O)C(C)C)n2)c(OC(C)C)cc1C1=C[C@@H](C2CCC2)N(C)[C@@H](C2CCC2)C1] | 5-chloro-N²-(4-((cis)-2,6-dicyclobutyl-1-methyl-1, 2,3,6-tetrahydropyridin-4-yl)-2-isopropoxy-5- methylphenyl)-N⁴-(2-(isopropylsulfonyl)phenyl) pyrimidine-2,4-diamine; |
| 9 | 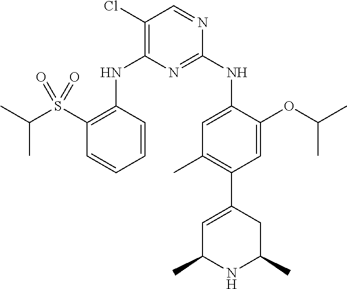 [SMILES: Cc1cc(Nc2ncc(Cl)c(Nc3ccccc3S(=O)(=O)C(C)C)n2)c(OC(C)C)cc1C1=C[C@H](C)N[C@H](C)C1] | 5-chloro-N²-(4-((cis)-2,6-dimethyl-1,2,3,6- tetrahydropyridin-4-yl)-2-isopropoxy-5-methylphenyl)- N⁴-(2-(isopropylsulfonyl)phenyl)pyrimidine-2,4- diamine; |
| 10 | 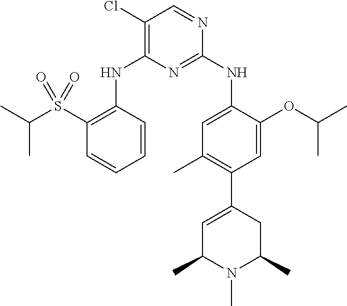 [SMILES: Cc1cc(Nc2ncc(Cl)c(Nc3ccccc3S(=O)(=O)C(C)C)n2)c(OC(C)C)cc1C1=C[C@H](C)N(C)[C@H](C)C1] | 5-chloro-N²-(2-isopropoxy-5-methyl-4-((cis)-1,2,6- trimethyl-1,2,3,6-tetrahydropyridin-4-yl)phenyl)- N⁴-(2-(isopropylsulfonyl)phenyl)pyrimidine-2,4- diamine; |
| 11 | 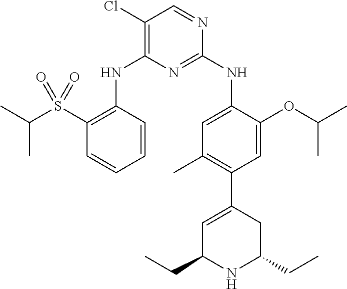 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1] | 5-chloro-N²-(4-((trans)-2,6-diethyl-1,2,3,6- tetrahydropyridin-4-yl)-2-isopropoxy-5-methylphenyl)- N⁴-(2-(isopropylsulfonyl)phenyl)pyrimidine-2,4- diamine; |
| 12 | 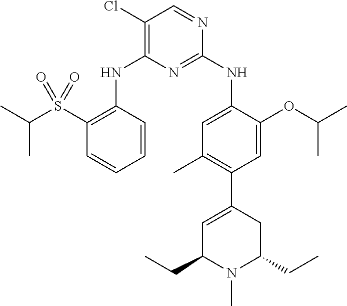 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1C] | 5-chloro-N²-(4-((trans)-2,6-diethyl-1-methyl-1,2,3, 6-tetrahydropyridin-4-yl)-2-isopropoxy-5- methylphenyl)-N⁴-(2-(isopropylsulfonyl)phenyl) pyrimidine-2,4-diamine; |
| 13 | 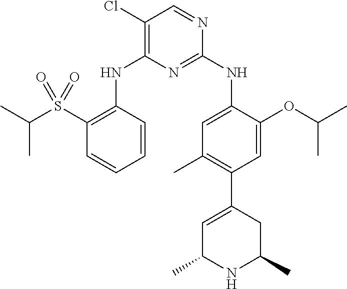 [SMILES: Cc1cc(Nc2ncc(Cl)c(Nc3ccccc3S(=O)(=O)C(C)C)n2)c(OC(C)C)cc1C1=C[C@@H](C)N[C@H](C)C1] | 5-chloro-N²-(4-((trans)-2,6-dimethyl-1,2,3,6- tetrahydropyridin-4-yl)-2-isopropoxy-5-methylphenyl)- N⁴-(2-(isopropylsulfonyl)phenyl)pyrimidine-2,4- diamine; |
| 14 | 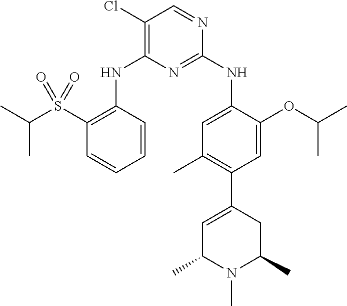 [SMILES: Cc1cc(Nc2ncc(Cl)c(Nc3ccccc3S(=O)(=O)C(C)C)n2)c(OC(C)C)cc1C1=C[C@@H](C)N(C)[C@H](C)C1] | 5-chloro-N²-(2-isopropoxy-5-methyl-4-((trans)-1,2,6- trimethyl-1,2,3,6-tetrahydropyridin-4-yl)phenyl)- N⁴-(2-(isopropylsulfonyl)phenyl)pyrimidine-2,4- diamine; |
| 15 | 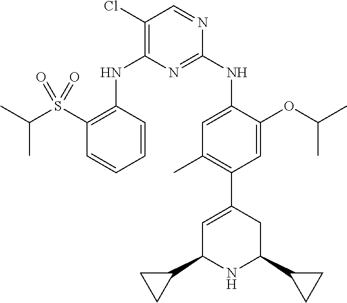 [SMILES: Cc1cc(Nc2ncc(Cl)c(Nc3ccccc3S(=O)(=O)C(C)C)n2)c(OC(C)C)cc1C1=C[C@H](C2CC2)N[C@H](C2CC2)C1] | 5-chloro-N²-(4-((cis)-2,6-dicyclopropyl-1,2,3,6- tetrahydropyridin-4-yl)-2-isopropoxy-5-methylphenyl)- N⁴-(2-(isopropylsulfonyl)phenyl)pyrimidine-2,4- diamine; |
| 16 | 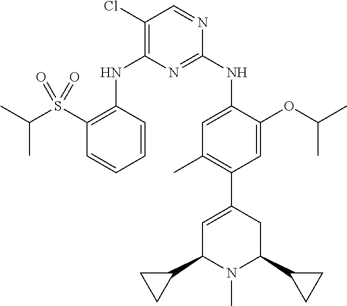 [SMILES: Cc1cc(Nc2ncc(Cl)c(Nc3ccccc3S(=O)(=O)C(C)C)n2)c(OC(C)C)cc1C1=C[C@H](C2CC2)N(C)[C@H](C2CC2)C1] | 5-chloro-N²-(4-((cis)-2,6-dicyclopropyl-1-methyl- 1,2,3,6-tetrahydropyridin-4-yl)-2-isopropoxy-5- methylphenyl)-N⁴-(2-(isopropylsulfonyl)phenyl) pyrimidine-2,4-diamine; |
| 17 | 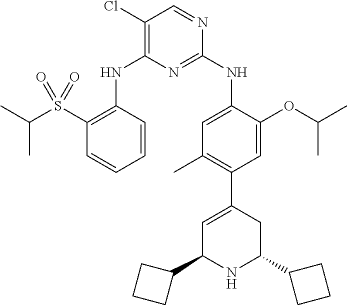 [SMILES: Cc1cc(Nc2ncc(Cl)c(Nc3ccccc3S(=O)(=O)C(C)C)n2)c(OC(C)C)cc1C1=C[C@H](C2CCC2)N[C@@H](C2CCC2)C1] | 5-chloro-N²-(4-((trans)-2,6-dicyclobutyl-1,2,3,6- tetrahydropyridin-4-yl)-2-isopropoxy-5-methylphenyl)- N⁴-(2-(isopropylsulfonyl)phenyl)pyrimidine-2, 4-diamine; |
| 18 | 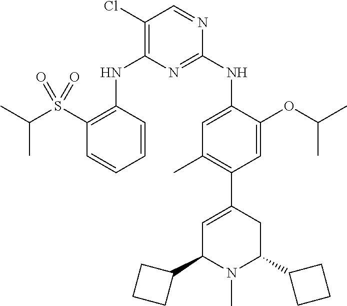 [SMILES: Cc1cc(Nc2ncc(Cl)c(Nc3ccccc3S(=O)(=O)C(C)C)n2)c(OC(C)C)cc1C1=C[C@H](C2CCC2)N(C)[C@@H](C2CCC2)C1] | 5-chloro-N²-(4-((trans)-2,6-dicyclobutyl-1-methyl- 1,2,3,6-tetrahydropyridin-4-yl)-2-isopropoxy-5- methylphenyl)-N⁴-(2-(isopropylsulfonyl)phenyl) pyrimidine-2,4-diamine; |
| 19 | 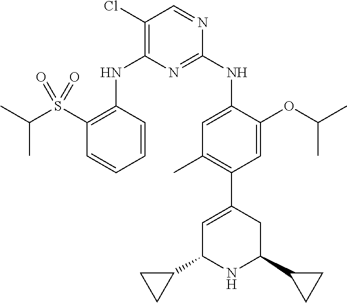 [SMILES: Cc1cc(Nc2ncc(Cl)c(Nc3ccccc3S(=O)(=O)C(C)C)n2)c(OC(C)C)cc1C1=C[C@@H](C2CC2)N[C@H](C2CC2)C1] | 5-chloro-N²-(4-((trans)-2,6-dicyclopropyl-1,2,3,6- tetrahydropyridin-4-yl)-2-isopropoxy-5-methylphenyl)- N⁴-(2-(isopropylsulfonyl)phenyl)pyrimidine-2, 4-diamine; |
| 20 | 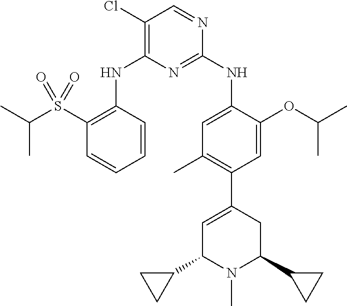 [SMILES: Cc1cc(Nc2ncc(Cl)c(Nc3ccccc3S(=O)(=O)C(C)C)n2)c(OC(C)C)cc1C1=C[C@@H](C2CC2)N(C)[C@H](C2CC2)C1] | 5-chloro-N²-(4-((trans)-2,6-dicyclopropyl-1-methyl- 1,2,3,6-tetrahydropyridin-4-yl)-2-isopropoxy-5- methylphenyl)-N⁴-(2-(isopropylsulfonyl)phenyl) pyrimidine-2,4-diamine; |
| 21 | 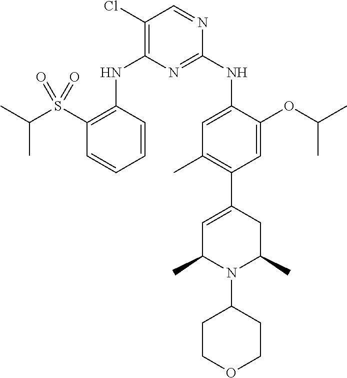 [SMILES: Cc1cc(Nc2ncc(Cl)c(Nc3ccccc3S(=O)(=O)C(C)C)n2)c(OC(C)C)cc1C1=C[C@H](C)N(C2CCOCC2)[C@H](C)C1] | 5-chloro-N²-(4-((cis)-2,6-dimethyl-1-(tetrahydro- 2H-pyran-4-yl)-1,2,3,6-tetrahydropyridin-4-yl)-2- isopropoxy-5-methylphenyl)-N⁴-(2-(isopropylsulfonyl) phenyl)pyrimidine-2,4-diamine; |
| 22 | 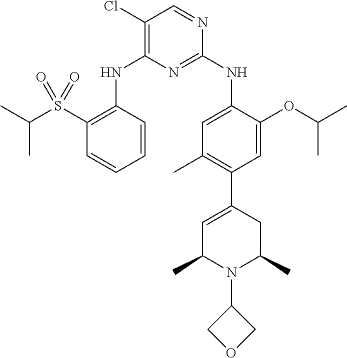 [SMILES: Cc1cc(Nc2ncc(Cl)c(Nc3ccccc3S(=O)(=O)C(C)C)n2)c(OC(C)C)cc1C1=C[C@H](C)N(C2COC2)[C@H](C)C1] | 5-chloro-N²-(4-((cis)-2,6-dimethyl-1-(oxetan-3-yl)- 1,2,3,6-tetrahydropyridin-4-yl)-2-isopropoxy-5- methylphenyl)-N⁴-(2-(isopropylsulfonyl)phenyl) pyrimidine-2,4-diamine; |
| 23 | 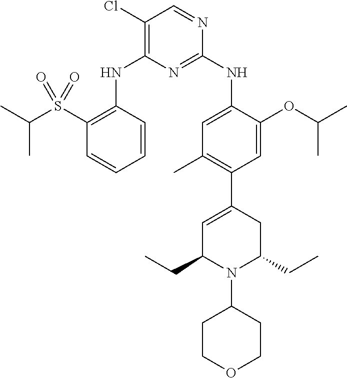 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1C1CCOCC1] | 5-chloro-N²-(4-((trans)-2,6-diethyl-1-(tetrahydro- 2H-pyran-4-yl)-1,2,3,6-tetrahydropyridin-4-yl)-2- isopropoxy-5-methylphenyl)-N⁴-(2-(isopropylsulfonyl) phenyl)pyrimidine-2,4-diamine; |
| 24 | 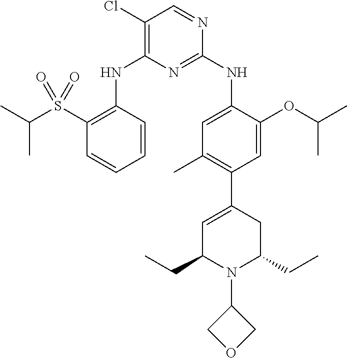 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1C1COC1] | 5-chloro-N²-(4-((2S,6S)-2,6-diethyl-1-(oxetan-3-yl)- 1,2,3,6-tetrahydropyridin-4-yl)-2-isopropoxy-5- methylphenyl)-N⁴-(2-(isopropylsulfonyl)phenyl) pyrimidine-2,4-diamine; |
| 25 | 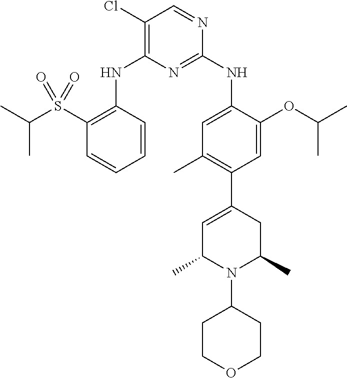 [SMILES: Cc1cc(Nc2ncc(Cl)c(Nc3ccccc3S(=O)(=O)C(C)C)n2)c(OC(C)C)cc1C1=C[C@@H](C)N(C2CCOCC2)[C@H](C)C1] | 5-chloro-N²-(4-((trans)-2,6-dimethyl-1-(tetrahydro- 2H-pyran-4-yl)-1,2,3,6-tetrahydropyridin-4-yl)-2- isopropoxy-5-methylphenyl)-N⁴-(2-(isopropylsulfonyl) phenyl)pyrimidine-2,4-diamine; |
| 26 | 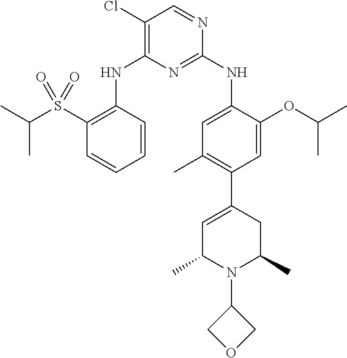 [SMILES: Cc1cc(Nc2ncc(Cl)c(Nc3ccccc3S(=O)(=O)C(C)C)n2)c(OC(C)C)cc1C1=C[C@@H](C)N(C2COC2)[C@H](C)C1] | 5-chloro-N²-(4-((trans)-2,6-dimethyl-1-(oxetan-3-yl)- 1,2,3,6-tetrahydropyridin-4-yl)-2-isopropoxy-5- methylphenyl)-N⁴-(2-(isopropylsulfonyl)phenyl) pyrimidine-2,4-diamine; |
| 27 | 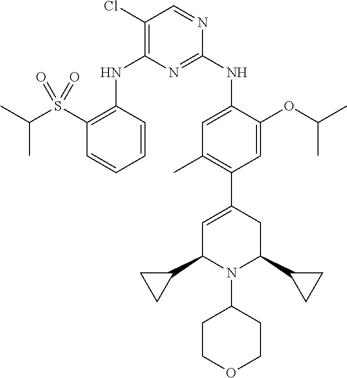 [SMILES: Cc1cc(Nc2ncc(Cl)c(Nc3ccccc3S(=O)(=O)C(C)C)n2)c(OC(C)C)cc1C1=C[C@H](C2CC2)N(C2CCOCC2)[C@H](C2CC2)C1] | 5-chloro-N²-(4-((cis)-2,6-dicyclopropyl-1-(tetrahydro- 2H-pyran-4-yl)-1,2,3,6-tetrahydropyridin-4-yl)- 2-isopropoxy-5-methylphenyl)-N⁴-(2- (isopropylsulfonyl)phenyl)pyrimidine-2,4-diamine; |
| 28 | 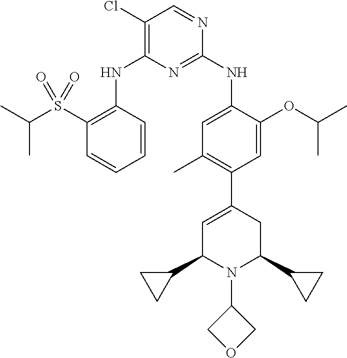 [SMILES: Cc1cc(Nc2ncc(Cl)c(Nc3ccccc3S(=O)(=O)C(C)C)n2)c(OC(C)C)cc1C1=C[C@H](C2CC2)N(C2COC2)[C@H](C2CC2)C1] | 5-chloro-N²-(4-((cis)-2,6-dicyclopropyl-1-(oxetan- 3-yl)-1,2,3,6-tetrahydropyridin-4-yl)-2-isopropoxy- 5-methylphenyl)-N⁴-(2-(isopropylsulfonyl)phenyl) pyrimidine-2,4-diamine; |
| 29 | 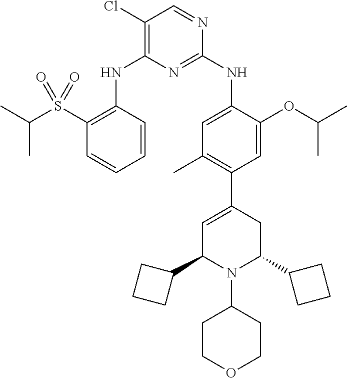 [SMILES: Cc1cc(Nc2ncc(Cl)c(Nc3ccccc3S(=O)(=O)C(C)C)n2)c(OC(C)C)cc1C1=C[C@H](C2CCC2)N(C2CCOCC2)[C@@H](C2CCC2)C1] | 5-chloro-N²-(4-((trans)-2,6-dicyclobutyl-1-(tetrahydro- 2H-pyran-4-yl)-1,2,3,6-tetrahydropyridin-4-yl)- 2-isopropoxy-5-methylphenyl)-N⁴-(2- (isopropylsulfonyl)phenyl)pyrimidine-2,4-diamine; |
| 30 | 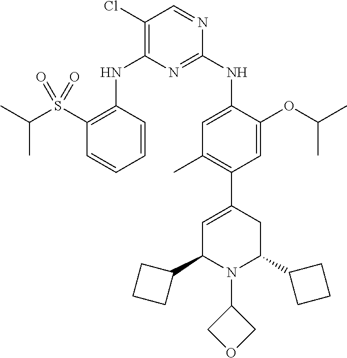 [SMILES: Cc1cc(Nc2ncc(Cl)c(Nc3ccccc3S(=O)(=O)C(C)C)n2)c(OC(C)C)cc1C1=C[C@H](C2CCC2)N(C2COC2)[C@@H](C2CCC2)C1] | 5-chloro-N²-(4-((trans)-2,6-dicyclobutyl-1-(oxetan- 3-yl)-1,2,3,6-tetrahydropyridin-4-yl)-2-isopropoxy- 5-methylphenyl)-N⁴-(2-(isopropylsulfonyl) phenyl)pyrimidine-2,4-diamine; |
| 31 | 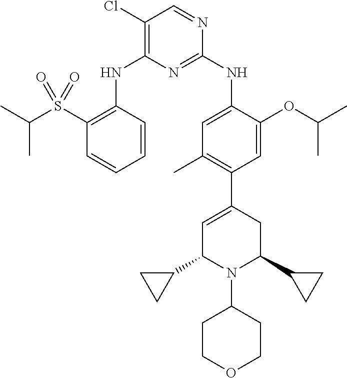 [SMILES: Cc1cc(Nc2ncc(Cl)c(Nc3ccccc3S(=O)(=O)C(C)C)n2)c(OC(C)C)cc1C1=C[C@@H](C2CC2)N(C2CCOCC2)[C@H](C2CC2)C1] | 5-chloro-N²-(4-((trans)-2,6-dicyclopropyl-1- (tetrahydro-2H-pyran-4-yl)-1,2,3,6-tetrahydropyridin- 4-yl)-2-isopropoxy-5-methylphenyl)-N⁴-(2-(isopropyl sulfonyl)phenyl)pyrimidine-2,4-diamine; or |
| 32 | 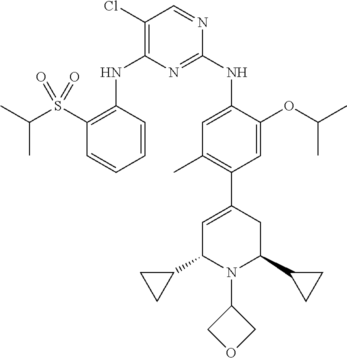 [SMILES: Cc1cc(Nc2ncc(Cl)c(Nc3ccccc3S(=O)(=O)C(C)C)n2)c(OC(C)C)cc1C1=C[C@@H](C2CC2)N(C2COC2)[C@H](C2CC2)C1] | 5-chloro-N²-(4-((trans)-2,6-dicyclopropyl-1-(oxetan- 3-yl)-1,2,3,6-tetrahydropyridin-4-yl)-2-isopropoxy- 5-methylphenyl)-N⁴-(2-(isopropylsulfonyl) phenyl)pyrimidine-2,4-diamine |

In a preferred embodiment, the ALK inhibitor is 5-chloro-N²-(2-isopropoxy-5 -methyl-4-(1-(tetrahydro-2H-pyran-4-yl)- 1,2,3,6-t etrahydropyridin-4-yl)phenyl)-N⁴-(2-(isopropylsulfonyl)phenyl)pyrimidine-2,4-diamine, or a pharmaceutically acceptable salt or hydrate thereof.

In a preferred embodiment, the CDK4/6 inhibitor is compound of the formulae (VII) or (VIII), or pharmaceutically acceptable salt thereof:

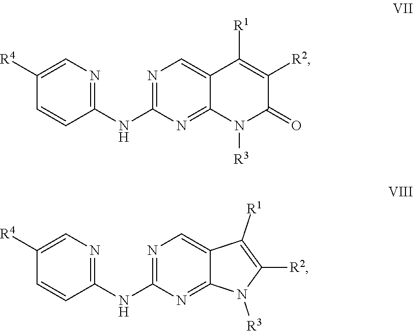

wherein in formulae (VII) or (VIII),
R¹ is selected from the group consisting of hydrogen, C₁-C₄ alkyl, and C₃-C₆ cycloalkyl;
R² is selected from the group consisting of hydrogen, C₁-C₄ alkyl, COR⁵, and CONR⁵R⁶;
R3 is C₃-C₆ cycloalkyl;
R⁴ is 4- to 6-membered heterocyclyl;
R⁵ and R⁶ are independently selected from hydrogen and C₁-C₄ alkyl.

In a preferred embodiment, the CDK4/6 inhibitor is palbociclib, ribociclib or a pharmaceutically acceptable salt thereof.

In a preferred embodiment, the Mek inhibitor is compound of the formulae (IX) or (X), or pharmaceutically acceptable salt thereof:

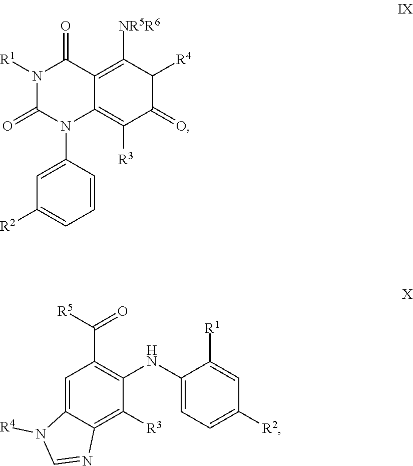

wherein in formulae (IX),
R¹ is selected from the group consisting of hydrogen, C₁-C₄ alkyl, and C₃-C₆ cycloalkyl;
R² is NHC(O)R⁷;
R³, R⁴, R⁵ and R⁷ are independently selected from the group consisting of hydrogen and C₁-C₄ alkyl;
R⁶ is selected from the group consisting of hydrogen, C₆-C₁₀ aryl group, wherein the aryl group is optionally substituted with one or two halogen groups;
wherein in formulae (X),
R¹ is selected from the group consisting of C₁-C₄ alkyl and halogen;
R² is selected from the group consisting of —OCF₃ and halogen;
R³ is selected from the group consisting of hydrogen and halogen;
R⁴ is selected from the group consisting of C₁-C₄ alkyl, and C₃-C₆ cycloalkyl;
R⁵ is NR⁶OR⁷, wherein R⁶ and R⁷ are independently selected from the group consisting of hydrogen and C₁-C₄ alkyl, and when R⁷ is C₁-C₄ alkyl, it is optionally substituted with hydroxy group.

In a preferred embodiment, the Mek inhibitor is trametinib, selumetinib or a pharmaceutically acceptable salt thereof.

In a preferred embodiment, the chemotherapeutic agent comprises platinum or belongs to terpenoid alkaloid.

In a preferred embodiment, the chemotherapeutic agent is carboplatin or paclitaxel.

In a preferred embodiment, the MDM2 inhibitor is compound of the formula (XI) or pharmaceutically acceptable salt thereof:

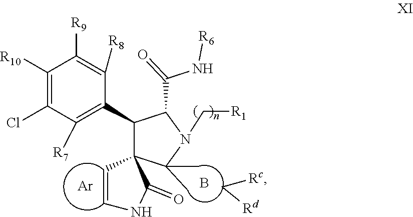

wherein,

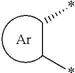

is

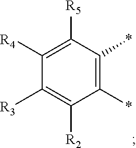

### B is

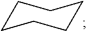

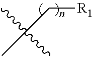

is hydrogen, CH₃, CH₂CH₃, C₃ alkyl or C₄ alkyl;
R₂ is hydrogen; R₃ is halogen; R₄ and R₅ are hydrogen;
R₆ is

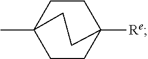

R₇ is halogen; each of R₈, R₉, and R₁₀ is H;
R^{e} is —C(O)OH, —C(O)NH₂, or —C(O)NHSO₂CH₃.

In a preferred embodiment, the MDM2 inhibitor is Compound 33 with the following structure including any tautomer forms, or a pharmaceutically acceptable salt thereof:

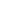

In a preferred embodiment, the HDAC inhibitor is compound of the formula (XII) or pharmaceutically acceptable salt thereof:

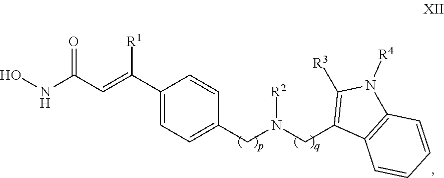

wherein,
R¹ and R⁴ are independently selected from the group consisting of hydrogen and C₁-C₄ alkyl;
R² is selected from the group consisting of hydrogen, —CH₂OH, —CH₂CH₂OH, and —CH₂CH₂CH₂OH;
R³ is selected from the group consisting of hydrogen, C₁-C₄ alkyl, and C₃-C₆ cycloalkyl;
p equals 1-3, and q equals 1-3.

In a preferred embodiment, the HDAC inhibitor is panobinostat or a pharmaceutically acceptable salt thereof.

In a preferred embodiment, the PD-1 inhibitor is anti-PD-1 antibody.

In a preferred embodiment, the PARP inhibitor is compound of the formula (XIII) or pharmaceutically acceptable salt thereof:

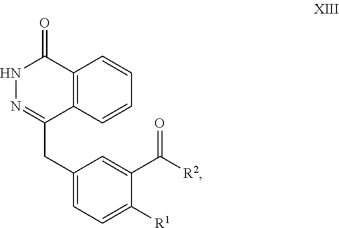

wherein,
R¹ is selected from the group consisting of hydrogen, C₁-C₄ alkyl, and halogen;
R² is 4- to 8-membered heterocyclyl, which is optionally substituted with —C(O)R³, wherein R³ is C₃-C₆ cycloalkyl.

In a preferred embodiment, the PARP inhibitor is Olaparib or a pharmaceutically acceptable salt thereof.

In a preferred embodiment, the VEGF inhibitor is compound of the formula (XIV) or pharmaceutically acceptable salt thereof:

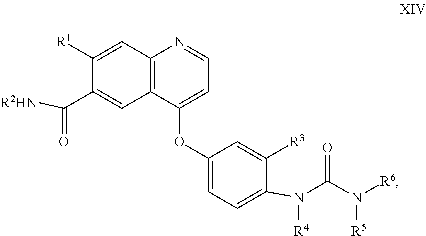

wherein,
R¹ is selected from the group consisting of C₁-C₄ alkyl, and C₁-C₄ alkyloxy;
R², R⁴ and R⁵ are independently selected from the group consisting of hydrogen, and C₁-C₄ alkyl;
R³ is selected from the group consisting of halogen, and C₁-C₄ alkyl;
R⁶ is selected from the group consisting of C₁-C₄ alkyl, and C₃-C₆ cycloalkyl.

In a preferred embodiment, the VEGF inhibitor is Lenvatinib or a pharmaceutically acceptable salt thereof.

In a preferred embodiment, the BCR-ABL inhibitor is compound of the formula (XV) or pharmaceutically acceptable salt thereof:

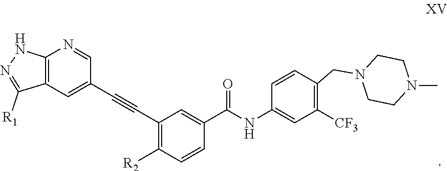

wherein,
R₁ is hydrogen, C₁-C₄ alkyl, C₃-C₆ cycloalkyl, C₁-C₄ alkyloxy, or phenyl; and R₂ is hydrogen, C₁-C₄ alkyl, C₃-C₆ cycloalkyl, or halogen.

In a preferred embodiment, the BCR-ABL inhibitor is Compound 34 with the following structure including any tautomer forms, or a pharmaceutically acceptable salt thereof:

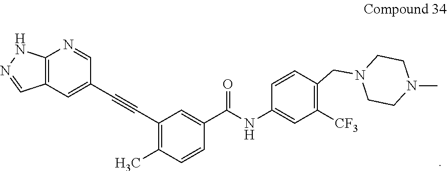 [SMILES: Cc1ccc(C(=O)Nc2ccc(CN3CCN(C)CC3)c(C(F)(F)F)c2)cc1C#Cc1cnc2[nH]ncc2c1]

In a preferred embodiment, the pharmaceutical composition is for treating or suppressing a cancer, reducing its severity, lowering its risk or inhibiting its metastasis in an individual, and the cancer is selected from the group consisting of bladder cancer, breast cancer, cervical cancer, colon cancer (including colorectal cancer), esophageal cancer, esophageal squamous cell carcinoma, head and neck cancel; liver cancer, lung cancer (including small cell lung cancer and non-small cell lung cancer), melanoma, myeloma, rhabdomyosarcoma, inflammatory myofibroblastic tumor, neuroturbo chargeoma, pancreatic cancer, prostate cancel; kidney cancer, renal cell carcinoma, sarcoma (including osteosarcoma), skin cancer (including squamous cell carcinoma), gastric cancer, testicular cancer, thyroid cancer, uterine cancel; mesothelioma, neuroblastoma, cholangiocarcinoma, leiomyosarcoma, lipo sarcoma, nasopharyngeal carcinoma, neuroendocrine carcinoma, ovarian cancer, salivary gland cancer, metastasis caused by spindle cell carcinoma, anaplastic large cell lymphoma, thyroid undifferentiated carcinoma, non-Hodgkin's lymphoma, Hodgkin's lymphoma, and hematological malignancies, such as acute myeloid leukemia (AML), acute lymphoblastic leukemia (ALL), diffuse large B-cell lymphoma (DLBCL), follicular lymphoma (FL), chronic lymphocytic leukemia (CLL), chronic myeloid leukemia (CML), uveal melanoma, glioblastoma;

In a preferred embodiment, the weight ratio between the ALK inhibitor and the one or more anticancer reagents is 0.005-5000: 0.005-5000, for example, 0.05-1500:0.005-5000, 0.1-6:0.005-4, 100:0.5-400, 100:1-350, 100:2-300, 100:5-200, 100:10-150, 100:20-100, 100:30-90, 100:20-80.

In some preferred embodiment, the cancer is mesothelioma, neuroblastoma, non-small cell lung cancer, lung adenocarcinoma (LUAD), ovarian cancer, uveal melanoma, glioblastoma, colon cancer, and liver cancer.

In a preferred embodiment, the ALK inhibitor is administrated in an amount of from about 0.005 mg/day to about 5000 mg/day, such as an amount of about 0.005, 0.05, 0.5, 5, 10, 20, 30, 40, 50, 100, 150, 200, 250, 300, 350, 400, 450, 500, 550, 600, 650, 700, 750, 800, 850, 900, 950, 1000, 1500, 2000, 2500, 3000, 3500, 4000, 4500 or 5000 mg/day.

In a preferred embodiment, the ALK inhibitor is administrated in an amount of from about 1 ng/kg to about 200 mg/kg, about 1 μg/kg to about 100 mg/kg, or about 1 mg/kg to about 50 mg/kg per unit dose, for example, administrated in an amount of about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg per unit dose, and administrated with one or more (e.g., 1, 2, 3, 4, 5, 6, 7, 8, 9 or 10) unit doses per day.

In a preferred embodiment, the one or more anticancer reagents are administrated in an amount of from 0.005 mg/day to about 5000 mg/day, for example, about 0.005, 0.05, 0.5, 5, 10, 20, 30, 40, 50, 100, 150, 200, 250, 300, 350, 400, 450, 500, 550, 600, 650, 700, 750, 800, 850, 900, 950, 1000, 1500, 2000, 2500, 3000, 3500, 4000, 4500 or 5000 mg/day.

In a preferred embodiment, the one or more anticancer reagents are administrated in an amount of from about 1 ng/kg to about 200 mg/kg, from about 1 μg/kg to about 100 mg/kg, or from about 1 mg/kg to about 50 mg/kg per unit dose, for example, administrated in an amount of about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg /kg, about 70 mg/kg, about 80 mg/kg, about 90 mg/kg, about 100 mg/kg, about 125 mg/kg, about 150 mg/kg, about 175 mg/kg, about 200 mg/kg per unit dose, and administered with one or more (e.g., 1, 2, 3, 4, 5, 6, 7, 8, 9, or 10) unit doses per day.

In a preferred embodiment, the ALK inhibitor, and the one or more anticancer reagents are administered together, simultaneously, sequentially or alternately.

In a preferred embodiment, the ALK inhibitor, and the one or more anticancer reagents are administered continuously for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days, or at least 50 days.

In a preferred embodiment, the ALK inhibitor, and the one or more anticancer reagents are administered for one or more (e.g., 1, 2, 3, 4, 5, 6, 7, 8, 9 or 10) courses of treatment, in which each of the courses lasts at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days, or at least 50 days; and there is an interval of 0, 1, 2, 3, 4, 5, 6, 7, 8, 9, 10 days, two weeks, three weeks or four weeks between every two courses of treatment.

In a preferred embodiment, when there are a plurality of courses of treatment, the amount of the ALK inhibitor and/or anticancer reagents administered in each course of treatment is same or different. In a more preferred embodiment, the amount of the ALK inhibitor and/or anticancer reagents administered during the previous course of treatment is 1-10 times, preferably 1-5 times, such as 1.5, 2, 2.5, 3, 3.5, 4, 4.5 or 5 times, the amount administered during the subsequent course of treatment.

In a preferred embodiment, the ALK inhibitor, and the one or more anticancer reagents are administrated via the same (e.g., oral) or different routes (e.g., oral and parenteral (e.g., injection), respectively).

In a preferred embodiment, the anticancer reagent is administrated in a lower dose in comparison with the dose of the anticancer reagent that is administered alone or when the one or more ALK inhibitors are not administered.

In a preferred embodiment, the ALK inhibitor enhances the therapeutic efficacy of the anticancer reagent in treatment of a cancer and/or reduces a side-effect of the anticancer reagent in treatment of a cancer.

In a preferred embodiment, the invention provides a use of an ALK inhibitor in manufacture of a medicament for enhancing the efficacy of an anticancer reagent in treatment of a cancer and/or reducing a side-effect of an anticancer reagent in treatment of a cancer.

In a preferred embodiment, the individual suffers from an advanced cancer.

In a preferred embodiment, the individual suffers from a refractory cancer, a recurrent cancer or a drug-resistant cancer.

In another embodiment, the invention provides a method for treating or suppressing a cancer, reducing its severity, lowering its risk or inhibiting its metastasis in an individual, comprising administering to the individual a therapeutically effective amount of an ALK inhibitor, and a therapeutically effective amount of one or more anticancer reagents selected from a CDK4/6 inhibitor, an Mek inhibitor, a BRAF inhibitor a chemotherapeutic agent, an MDM2 inhibitor, an HDAC inhibitor, a PD-1 inhibitor, a PARP inhibitor, a VEGF inhibitor and a BCR-ABL inhibitor;

In a preferred embodiment, the ALK inhibitor is as defined above and the anticancer reagent is as defined above.

In another embodiment, the invention provides a use of a pharmaceutical composition in manufacture of a medicament for treating or suppressing a cancer, reducing its severity, lowering its risk or inhibiting its metastasis in an individual, the pharmaceutical composition comprising an ALK inhibitor, and one or more anticancer reagent, as well as optionally a pharmaceutically acceptable carrier;

In a preferred embodiment, the ALK inhibitor is as defined above and the anticancer reagent is as defined above.

In another embodiment, the invention provides a kit, comprising:
(a) a first component in a first container, the first component comprising an ALK inhibitor (preferably an ALK inhibitor as defined above), and optionally a pharmaceutically acceptable carrier;
(b) a second component in a second container, the second component comprising an anticancer reagent (preferably an anticancer reagent as defined above), and optionally a pharmaceutically acceptable carrier; and
(c) an optional specification.

### EXAMPLES

In order to make the objects and technical solutions of the present invention clearer, the present invention will be further described below in conjunction with specific example. It should be understood that the examples are not intended to limit the scope of the invention. Further, specific experimental methods not mentioned in the following examples were carried out in accordance with a conventional experimental method.

### Example 1. Cell Viability WST Assay—Combination Treatment with Compound 5 and Compound 34 in NCI-112228 Cells

The compounds of formulae (I) to (VI) or a pharmaceutically acceptable salt thereof including Compound 5 (5-chloro-N²-(2-isopropoxy-5-methyl-4-(1-(tetrahydro-2H-pyran-4-yl)-1,2,3,6-tetrahydropyridin-4-yl)phenyl)-N⁴-(2-(isopropylsulfonyl)phenyl)pyrimidine-2,4-diamine) are synthesized according to the production methods described in WO 2018/044767, which is incorporated herein by reference in its entirety and for all purposes, or a method analogous thereto.

The compounds of formula (XV) or a pharmaceutically acceptable salt thereof including Compound 34 can be obtained according to the production methods described in U.S. Pat. No. 8,846,671 B2, issued Sep. 30, 2014, which is incorporated herein by reference in its entirety and for all purposes, or a method analogous thereto.

Cell plating: Anti-proliferative effects were detected by a CCK-8 (Cell Counting Kit-8, Shanghai life iLab, China) assay based on water soluble tetrazolium salt (WST). The cells were seeded in 96-well plates, and only 95 μL of complete medium was added to each negative control group. 95 μL of complete medium cell suspension was added to each well to be tested, and the cell density was (5-10)×10\hat{ }4/hole.

Dosing (protection from light): In 96-well culture plates, according to the sensitivity of different cells to different drugs, the highest concentration was selected as 3.7 μM, and 6 concentrations were obtained by serial dilution in a ratio of 1:3. 5 μL of compound was added to each well and 2-3 replicate wells were made for per concentration. After the compound was added, 96-well plates were incubated in a 5% CO₂ incubator at 37° C. After 72 hours of action by using 6 different concentrations of Compound 34 with 3 fixed doses of Compound 5, the combination effect of Compound 5 an Compound 34 was tested.

Reading: At the end of the culture, the old solution was removed from the well to be tested, and 100 μl/well CCK-8 test solution (containing 10% CCK-8, 5% FBS in the corresponding medium) was added. The plates were continuously incubated at 37 ° C. for 2-4 hours in a CO₂ incubator.

The OD values were measured using a microplate reader (SpectraMax Plus 384, Molecular Devices, LLC., US) under A450 nm. Using the average OD value of 3 replicate wells, the percentage of cell viability was calculated by the following formula:

(O.D. of test well−O.D. of blank control well)/(O.D. of cell control well−O.D. of blank control well)×100%.

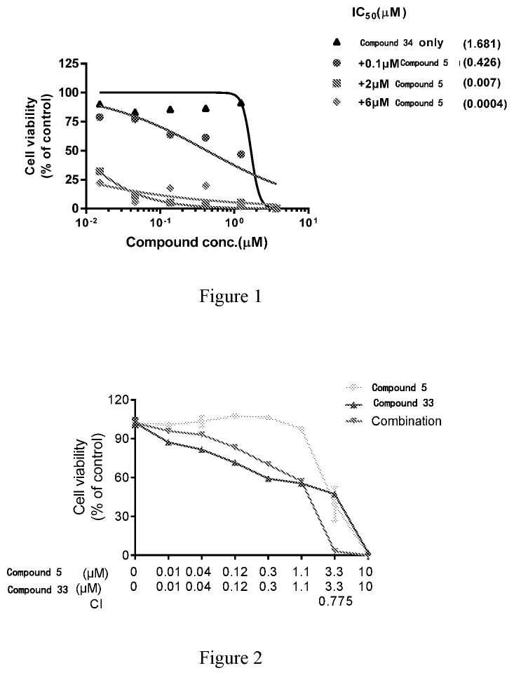

IC₅₀ values were calculated using Graphpad Prism 6.0 software for nonlinear regression data analysis method. The results are shown in FIG. 1 and Table 1.

For combination experiments, cell viability was calculated by normalization of the mean OD values of 3 replicate wells of single drug control. The comparison of the IC₅₀ values obtained from the curves of combined drugs of administration and single drug of administration shows that the two compounds achieved synergistic effect (the curve of the combined drugs of administration shifted to the left).

TABLE 1
Combination of Compound 5 and Compound 34 enhances
inhibition effect on NCI-H2228 tumor cell proliferation

| | | | | Combined | |
|---|---|---|---|---|---|
| | | | | admin- | |
| | | | | istration | IC₅₀ |
| | | | | IC₅₀ | (single |
| Compound | Compound | Compound | Compound | (A + B)* | B)/IC₅₀ |
| A | B | A IC₅₀ | B IC₅₀ (μM) | (μM) | (A + B) |
| Compound | Compound | 0.906 | 1.681 | 0.426/ | 4/240/ |
| 5 | 34 | | | 0.007/ | 4202 |
| | | | | 0.0004 | |

Notation: The three IC₅₀ values of the combinations respectively correspond to the concentration of Compound 5 at 0.1 μM, 2 μM and 6 μM, and the concentrations for Compound 34 are 6 concentrations obtained by serial dilution in a ratio of 1:3 with the highest concentration of 3.7 μM.

As shown in Table 1, Compound 34 alone or a combination of Compound 5 and Compound 34 was used to treat NCI-H2228 cells for 72 hours, and such combination shows a synergistic antiproliferative activity in this model.

### Example 2. Cell viability CTG Assay—Combination Treatment with Compound 5 and Compound 33 in Uveal Melanoma MP41 Cells

The compounds of formula (XI) or a pharmaceutically acceptable salt thereof including Compound 33 and any tautomer forms was prepared using the one or more procedures described in U.S. Pat. No. 9745314, and Aguilar et al. J. Med. Chem. 2017(60), 2819-2839.

Cell viability was determined using CellTiter-Glo® luminescent cell viability assay (Promega) by following manufacturer's instruction. Cell viability was calculated as cell viability=(mean RLU sample−mean RLU blank)/(RLU cell control−RLU blank)×100. IC₅₀ value was calculated using GraphPad Prism. Combination index (CI) value was calculated by CalcuSyn software (BIOSOFT, UK). CI<0.9 indicate a synergistic combination effect. CI<0.1 scored as 5+ indicates very strong synergistic combination effect, CI between 0.1 and 0.3 scored as 4+ indicates strong synergistic combination effect, CI between 0.3 and 0.7 scored as 3+ indicates medium synergistic combination effect.

Uveal melanoma MP41 cells (ATCC) were treated with Compound 5, MDM2 inhibitor Compound 33 or a combination of Compound 5 and Compound 33 respectively, wherein the culture of RPMI 1640+10% FBS (Fetal Bovine Serum)+1% P/S (Penicillin-Streptomycin) was used.

The results are shown in FIG. 2, wherein Compound 5 and Compound 33 demonstrated enhanced cell viability inhibition in Uveal melanoma MP41 cells, CI=0.775.

### Example 3. Evaluation Method for In Vivo Pharmacodynamic Experiment

A subcutaneous xenograft tumor model of human tumor immunodeficient mice was established by cell inoculation: tumor cells in logarithmic growth phase were collected, counted, resuspended in 1×PBS, and the cell suspension concentration was adjusted to 2.5-5 ×10⁷/mL. The tumor cells were inoculated subcutaneously in the right side of immunodeficient mice with a 1 mL syringe (4 gauge needle), 5-10×10⁶/0.2 mL/mouse. All animal experiments were strictly in accordance with the specifications for the use and management of experimental animals in GenePharma Co., Ltd. and Suzhou Ascentage Pharma Co., Ltd. The calculation of relevant parameters refers to the Chinese NMPA “Guidelines for Non-Clinical Research Techniques of Cytotoxic Anti-tumor Drugs”.

Animal body weight and tumor size were measured twice weekly during the experiment. The state of the animal and the presence or absence of death were observed every day. The growth of tumor and the effects of treatment on normal behavior of animals were monitored routinely, specifically involving experimental animal activity, feeding and drinking, weight gain or loss, eyes, clothing hair and other abnormalities. The deaths and clinical symptoms observed during the experiment were recorded in the raw data. All operations for administration and measurement of mouse body weight and tumor volume were performed in a clean bench. According to the requirements of the experimental protocol, after the end of the last administration, plasma and tumor tissues were collected, weighed and photographed. The plasma and tumor samples were frozen at −80° C. for ready-to-use.

Tumor volume (TV) is calculated as: TV=a×b²/2, wherein a and b represent the length and width of the tumor to be measured, respectively.

The relative tumor volume (RTV) is calculated as: RTV=Vₜ/V₁, wherein V₁ is the tumor volume at the start of grouping and administration, and Vₜ is the tumor volume measured on the t day after administration.

The evaluation index of anti-tumor activity is the relative tumor proliferation rate T/C (%), and the calculation formula thereof is: relative tumor proliferation rate T/C (%)=(T_{RTV}/C_{RTV})×100%, T_{RTV} is the RTV of treatment group, C_{RTV} is the RTV of solvent control group.

Tumor regression rate (%) is calculated as: the number of tumor-bearing mice which exhibit SD (stable disease), PR (partial regression) and CR (complete regression) after treatment/the total number of the mice in this group×100%.

Change of body weight (%)=(measured body weight−body weight at the start of grouping)/body weight at the start of grouping×100%.

Evaluation criteria for therapeutic efficiency: According to the Chinese NMPA “Guidelines for Non-Clinical Research Techniques of Cytotoxic Anti-tumor Drugs” (November 2006), when T/C (%) value is <40% and statistical analysis shows p<0.05, efficiency is confirmed. A dose of drug is considered to be severely toxic if the body weight of mouse is reduced by more than 20% or the number of drug-related deaths exceeds 20%.

According to the description by Clarke R., Issues in experimental design and endpoint analysis in the study of experimental cytotoxic agents in vivo in breast cancer and other models [J]. Breast Cancer Research & Treatment, 1997, 46(2-3): 255-278, synergy analysis was evaluated using the following formula: synergy factor=((A/C)×(B/C))/(AB/C); A=RTV value of drug A alone group; B=RTV value of drug B alone group; C=RTV value of the solvent control group, and AB=RTV value of the A and B combination group. Synergistic factor>1 indicates that synergy is achieved; synergy factor=1 indicates that additive effect is achieved; and synergy factor<1 indicates that antagonistic effect is achieved.

Use of mRECIST (Gao et al., 2015) measured tumor responses included stable disease (SD), partial tumor regression (PR), and complete regression (CR), determined by comparing tumor volume change at day t to its baseline: tumor volume change (%)=(Vₜ−V₁)/V₁. The BestResponse was the minimum value of tumor volume change (%) for t≥10. For each time t, the average of tumor volume changes from t=1 to t was also calculated. BestAvgResponse was defined as the minimum value of this average for t≥10. The criteria for response (mRECIST) were adapted from RECIST criteria (Gao et al., 2015; Therasse et al., 2000) and defined as follows: mCR, BestResponse<−95% and BestAvg Response<−40%; mPR, BestResponse<−50% and BestAvgResponse<−20%; mSD, BestResponse<35% and BestAvgResponse<30%; mPD, not otherwise categorized. SD, PR, and CR were considered responders and used to calculate response rate (%). Disease control rate (DCR) is calculated with the proportion of animals demonstrating CR, PR, or SD based on mRECIST; Overall response rate (ORR) is calculated with the proportion of animals demonstrating CR or PR based on mRECIST. Body weight of animals were monitored simultaneously. The change in body weight was calculated based on the animal weight of the first day of dosing (day 1). Tumor volume and changes in body weight (%) were represented as the mean±standard error of the mean (SEM).

The evaluation method as described in Example 3 is used in Examples 4-20.

### Example 4. Combination Treatment with Compound 5 and Palbociclib in Subcutaneous (s.c.) Mesothelioma MT16036 (TP53 mut, FAK Amp and CDK4 High) PDX Xenograft Tumor Model

In this experiment, a human MT16036 cell-derived xenograft (TP53 mut, FAK amp and CDK4 high) tumor model (Crown Bioscience) was established to evaluate the anti-tumor effect of Compound 5 in combination with CDK4/6 inhibitor palbociclib (Yishiming(Beijing) Pharm-Chemicals Tech. Co., Ltd). The dosing regimen was as follows:
Compound 5: 50 mg/kg, orally, once per day, for a total of 33 days;
Palbociclib: 50 mg/kg, orally, once per day, for a total of 33 days;

The dosing regimen of each drug in the dosing regimen for the combination is the same as the dosing regimen for the single drug.

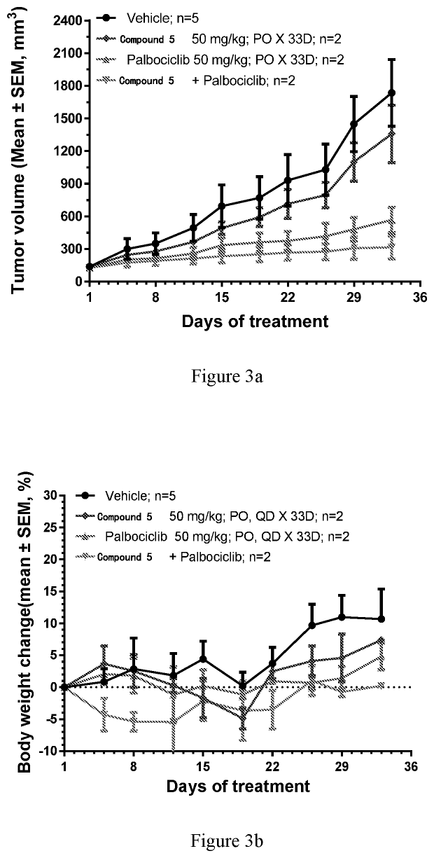

As shown in Table 2 and FIG. 3a, after 33 days of administration, the T/C (%) values of Compound 5, palbociclib and the combination of palbociclib with Compound 5 were 80.31%, 34.24% and 19.47% respectively. After 33 days of administration, the combination group had a synergistic factor of 1.41, indicating synergistic effects. Furthermore, animals from the combination group achieved ½ SD.

In addition, as shown in FIG. 3b, little body weight loss (less than 10%) was observed in the combination group during the study and the body weight was recovered at the end of study.

In conclusion, Compound 5 single agent showed minor antitumor activity, palbociclib single agent showed moderate antitumor activity, while the combination treatment showed enhanced antitumor activity, synergistic antitumor effect and acceptable toxicity in s.c. Mesothelioma PDX.

TABLE 2
Synergistic anti-tumor effect of Compound 5 in combination with palbociclib
in Mesothelioma MT16036 PDX mouse xenograft tumor model

| | RTV on | | | |
|---|---|---|---|---|
| | Day 33 after | T/C on Day 33 | Synergistic | Modified response |
| | administration | after | factor on Day | evaluation criteria |
| | (mean ± | administration | 33 after | in solid tumors |
| Treatment | standard error) | (%) | administration | (mRECIST) |
| Vehicle | 12.62 ± 0.24 | — | — | 2/2 PD |
| control | | | | |
| Compound 5 | 10.13 ± 2.97 | 80.31 | — | 2/2 PD |
| Palbociclib | 4.32 ± 0.69 | 34.24 | — | 2/2 PD |
| Compound 5 + | 2.45 ± 0.78 | 19.47 | 1.41 | 1/2 SD, 1/2 PD |
| palbociclib | | | | |

### Example 5. Combination treatment with Compound 5 and Ribociclib in s.c. Neuroblastoma SH-SY5Y (ALK F1174L) Xenograft Tumor Model

In this experiment, an Neuroblastoma SH-SY5Y (ALK F1174L) xenograft tumor model was established to evaluate the anti-tumor effect of Compound 5 in combination with CDK4/6 inhibitor ribociclib (Yishiming(Beijing) Pharm-Chemicals Tech. Co., Ltd). The dosing regimen was as follows:
Compound 5: 50 mg/kg, orally, once per day, for a total of 21 days;
Ribociclib: 75 mg/kg, orally, once per day, for a total of 21 days;

The dosing regimen of each drug in the dosing regimen for the combination is the same as the dosing regimen for the single drug.

The cell source is ATCC, and the cell culture is F12K: MEM=1:1 with sodium pyruvate (1×) and NEAA (1×), 90%; fetal bovine serum, 10%; P/S, 1%.

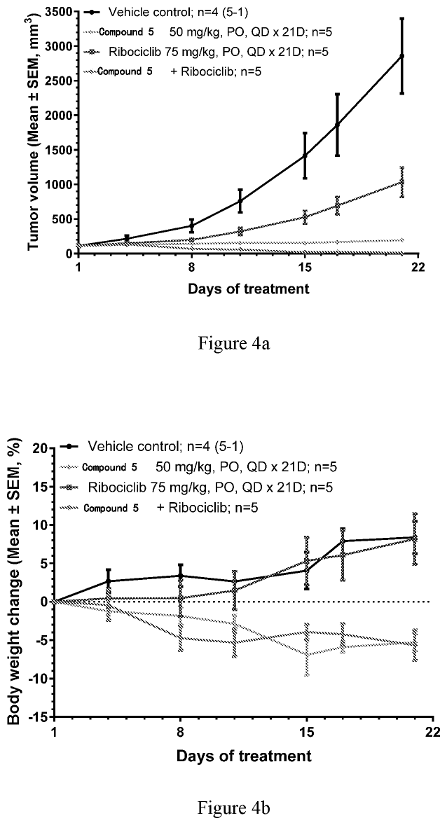

As shown in Table 3 and FIG. 4a, after 21 days of administration, the T/C (%) values of Compound 5, ribociclib and the combination of ribociclib with Compound 5 were 6.74%, 33.7% and 0.37% respectively. After 21 days of administration, the combination group had a synergistic factor of 6.07, indicating strong synergistic effects. Animals from combination group achieved ⅖ CR, ⅗ PR, ORR (overall response rate)=100%.

In addition, as shown in FIG. 4b, little body weight loss (less than 10%) was observed in Compound 5 single agent group and the combination group during the study.

In conclusion, ribociclib single agent showed moderate antitumor activity, Compound 5 single agent showed strong antitumor activity, while the combination treatment showed significantly enhanced tumor regression, strong synergistic antitumor effect and acceptable toxicity in s.c. Neuroblastoma SH-SY5Y xenograft.

TABLE 3
Synergistic anti-tumor effect of Compound 5 in combination with ribociclib in
Neuroblastoma SH-SY5Y (ALK F1174L) mouse xenograft tumor model

| | RTV on Day | | | | mRECIST on |
|---|---|---|---|---|---|
| | 21 after | | Synergistic | | Day 21 after |
| | administration | T/C on Day | factor | mRECIST | administration |
| | (mean ± | 21 after | on Day | (Disease | (Overall |
| | standard | administration | 21 after | control | regression |
| Treatment | error) | (%) | administration | rate, %) | rate, %) |
| Vehicle | 26.51 ± 4.39 | — | — | 5/5 PD | 5/5 PD |
| control | | | | | |
| Compound 5 | 1.79 ± 0.22 | 6.74 | — | 3/5 SD, | 2/5 SD, 3/5 PD |
| | | | | 2/5 PD (60%) | |
| Ribociclib | 8.93 ± 1.38 | 33.7 | — | 5/5 PD | 5/5 PD |
| Compound 5 + | 0.10 ± 0.05 ^{†§} | 0.37 | 6.07 | 2/5 CR, 3/5 | 2/5 CR, |
| ribociclib | | | | PR (100%) | 3/5 PR (100%) |

^{†}p < 0.05, compared to Compound 5 group, ^{§}p < 0.05, compared to ribociclib group

### Example 6. Combination Treatment with Compound 5 and Trametinib/Selumetinibin in s.c. A549 NSCLC Xenograft Tumor Model

In this experiment, a human A549 cell-derived xenograft lung tumor model was established to evaluate the anti-tumor effect of Compound 5 in combination with MEK inhibitor trametinib/selumetinib (Selleck). The dosing regimen was as follows:
Compound 5: 100 mg/kg, orally, once per day, for a total of 28 days;
Trametinib: 0.5 mg/kg, orally, once per day, for a total of 28 days; Selumetinib: 50 mg/kg, orally, once per day, for a total of 28 days;

The dosing regimen of each drug in the dosing regimen for the combination is the same as the dosing regimen for the single drug.

The cell source is Cobioer, and the cell culture is RPMI 1640 medium with 300 mg/L (2 mM) L-glutamine adjusted to contain 2.0 g/L sodium bicarbonate, 90%; fetal bovine serum, 10%; P/S 1%.

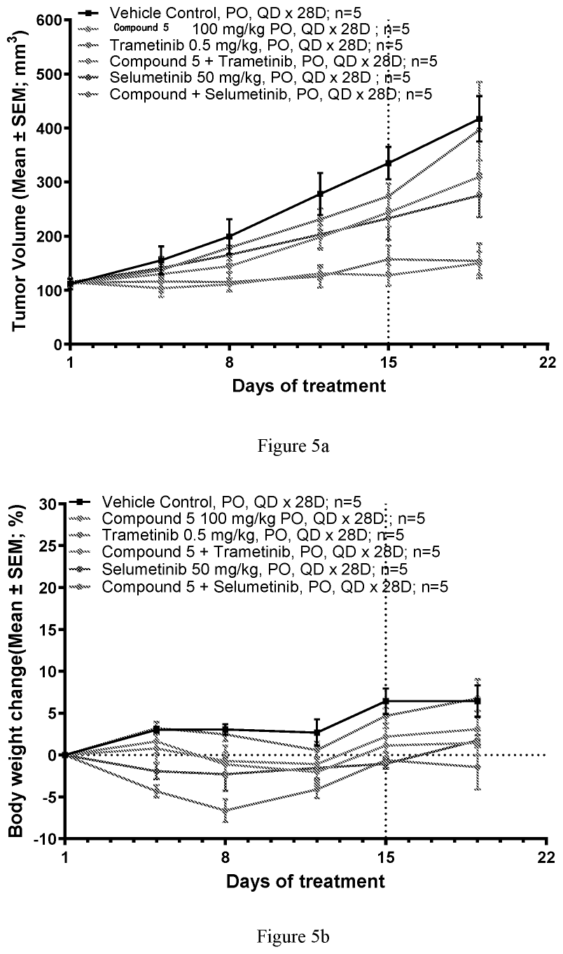

As shown in Table 4 and FIG. 5a, after 19 days of administration, the T/C (%) values of Compound 5, trametinib and the combination of trametinib with Compound 5 were 72.2%, 93.0% and 34.9% respectively. After 19 days of administration, the T/C (%) values of Compound 5, selumetinib and the combination of selumetinib with Compound 5 were 72.2%, 62.5% and 35.8% respectively, and the combination group had a synergy factor of 1.26, indicating synergistic effects.

In addition, as shown in FIG. 5b, little body weight loss (less than 10%) was observed in the combination groups during the study and the body weight was recovered at the end of study.

In conclusion, in A549 NSCLC xenograft, Compound 5 single agent or MEK inhibitor (trametinib or selumetinib) showed minor antitumor activity, while combination treatment of Compound 5 and MEK inhibitor (trametinib or selumetinib) showed significantly enhanced antitumor activity and synergistic antitumor effect in A549 NSCLC xenograft.

TABLE 4
Synergistic anti-tumor effect of Compound 5 in combination with trametinib/selumetinib
in A549 NSCLC mouse xenograft tumor model

| | RTV on Day | | | | |
|---|---|---|---|---|---|
| | 19 after | | Synergistic | | |
| | administration | T/C on | factor | | |
| | (mean ± | Day 19 after | on Day | | mRECIST on |
| | standard | administration | 19 after | | Day 19 after |
| Treatment | error) | (%) | administration | mRECIST | administration |
| Vehicle control | 3.8 ± 0.3 | — | — | 5/5 PD | 5/5 PD |
| Compound 5 | 2.7 ± 0.2 | 72.2 | — | 1/5 SD, 4/5 PD | 5/5 PD |
| Trametinib | 3.5 ± 1.0 | 93.0 | — | 5/5 PD | 5/5 PD |
| Compound 5 + | 1.3 ± 0.2** | 34.9 | 1.91 | 5/5 SD | 2/5 SD, 3/5 PD |
| trametinib | | | | | |
| Selumetinib | 2.4 ± 0.2 | 62.5 | — | 1/5 SD, 4/5 PD | 5/5 PD |
| Compound 5 + | 1.3 ± 0.3* | 35.8 | 1.26 | 3/5 SD, 2/5 PD | 3/5 SD, 2/5 PD |
| selumetinib | | | | | |

*P < 0.05, **P < 0.01, compared to vehicle control group

### Example 7. Combination Treatment with Compound 5 and Carboplatin in s.c. PTK2high NSCLC PDX Model of LU-01-0604

In this experiment, a PTK2 high NSCLC PDX model of LU-01-0604 (Wuxi Pharma Tech) was established to evaluate the anti-tumor effect of Compound 5 in combination with chemotherapeutic agent carboplatin (Selleck). The dosing regimen was as follows:
Compound 5: 50 mg/kg, orally, once per day, for a total of 29 days;
Carboplatin: 30 mg/kg, intraperitoneal injection, once per week, for a total of 29 days;

The dosing regimen of each drug in the dosing regimen for the combination is the same as the dosing regimen for the single drug.

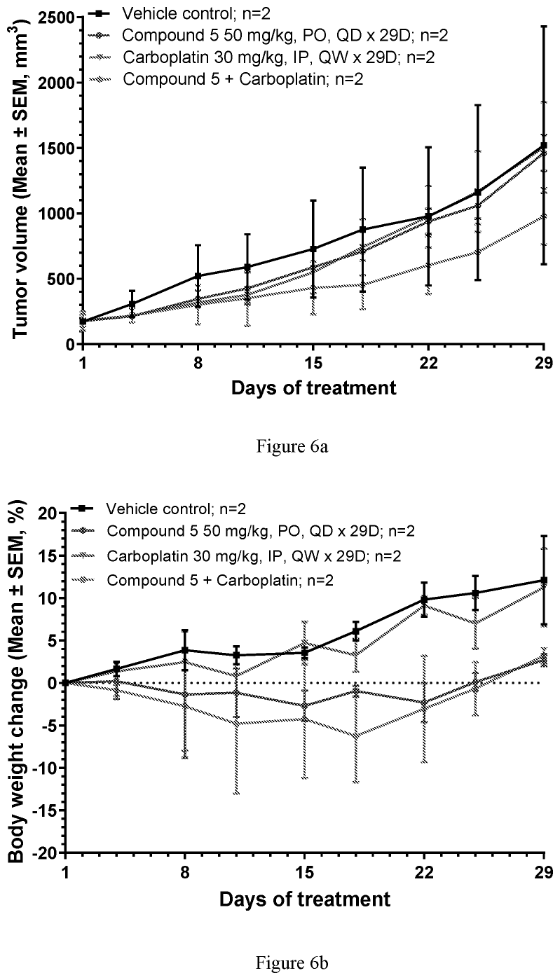

As shown in Table 5 and FIG. 6a, after 29 days of administration, the T/C (%) values of Compound 5, carboplatin and the combination of carboplatin with Compound 5 were 94.50%, 127.72% and 62.77% respectively.

After 29 days of administration, the combination group had a synergistic factor of 1.92, indicating synergistic effects.

In addition, as shown in FIG. 6b, little body weight loss (less than 10%) was observed in the combination groups during the study and the body weight was recovered at the end of study.

In conclusion, Compound 5 single agent and carboplatin single agent showed no antitumor activity, while the combination treatment showed enhanced antitumor activity and synergistic antitumor effect in s.c. NSCLC PDX.

TABLE 5
Synergistic anti-tumor effect of Compound 5 in combination
with carboplatin in PTK2 high NSCLC PDX mouse xenograft tumor
model

| | RTV on | T/C on | Synergistic | |
|---|---|---|---|---|
| | Day 29 after | Day 29 | factor on | |
| | administration | after admin- | Day 29 after | |
| | (mean ± | istration | admin- | |
| Treatment | standard error) | (%) | istration | mRECIST |
| Vehicle | 8.73 ± 5.18 | — | — | 2/2 PD |
| control | | | | |
| Compound 5 | 8.25 ± 1.03 | 94.50 | — | 2/2 PD |
| Carboplatin | 11.15 ± 6.55 | 127.72 | — | 1/2 SD, |
| | | | | 1/2 PD |
| Compound 5 + | 5.48 ± 0.26 | 62.77 | 1.92 | 1/2 SD, |
| carboplatin | | | | 1/2 PD |

n < 3, no statistical analysis applied

### Example 8. Combination Treatment with Compound 5 and Carboplatin in s.c. Ovarian PA-1 Xenograft Tumor Model

In this experiment, an ovarian PA-1 xenograft tumor model was established to evaluate the anti-tumor effect of Compound 5 in combination with chemotherapeutic agent carboplatin (Selleck). The dosing regimen was as follows:
Compound 5: 100 mg/kg, orally, once per day, for a total of 21 days;
Carboplatin: 50 mg/kg, intraperitoneal injection once on Day 1;

The dosing regimen of each drug in the dosing regimen for the combination is the same as the dosing regimen for the single drug.

The cell source is Cobioer, and the cell culture is MEM medium with fetal bovine serum of 10% and P/S of 1%.

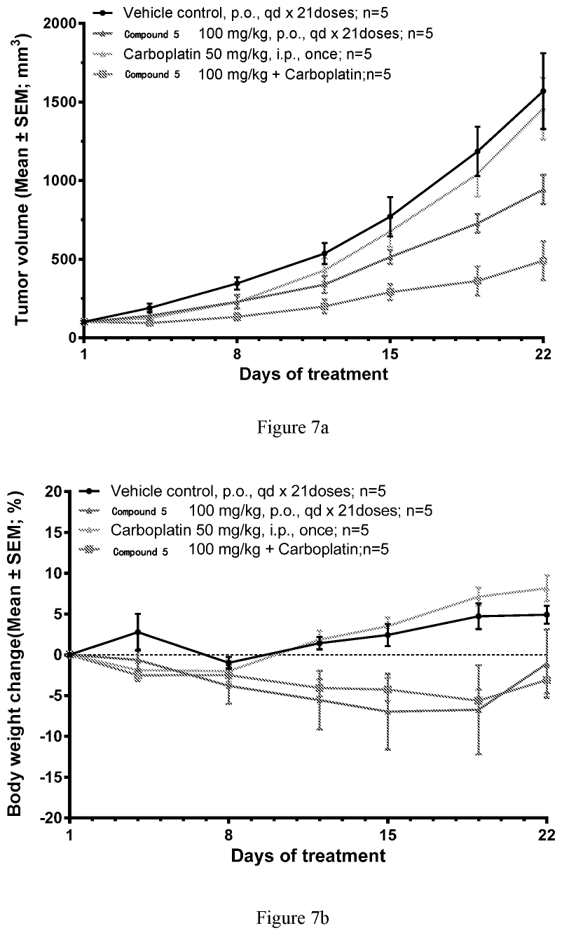

As shown in Table 6 and FIG. 7a, on Day 22, the T/C (%) values of Compound 5, carboplatin and the combination of carboplatin with Compound 5 were 62.89%, 94.97% and 30.82% respectively, and the synergy factor for the combination group was 1.94, indicating synergistic effects.

In addition, as shown in FIG. 7b, little body weight loss (not more than 5%) was observed in the combination groups during the study.

In conclusion, Compound 5 single agent showed moderate antitumor activity, while the combination treatment showed synergistic antitumor effect in s.c. PA-1 ovarian cancer xenografts.

TABLE 6
Synergistic anti-tumor effect of Compound 5 in combination
with carboplatin in ovarian PA-1 mouse xenograft tumor model

| | RTV on Day 22 | | |
|---|---|---|---|
| | after | T/C on Day 22 | Synergistic |
| | administration | after | factor on Day |
| | (mean ± | administration | 22 after |
| Treatment | standard error) | (%) | administration |
| Vehicle | 15.9 ± 2.0 | — | — |
| control | | | |
| Compound 5 | 10.0 ± 1.0 | 62.89 | — |
| Carboplatin | 15.1 ± 2.3 | 94.97 | — |
| Compound 5 + | 4.9 ± 1.2 ***^{+#} | 30.82 | 1.94 |
| carboplatin | | | |

***p < 0.001 compared to vehicle control group;
⁺p < 0.05 compared to Compound 5 group;
^{#}p < 0.05 compared to carboplatin group

### Example 9. Combination Treatment with Compound 5 and Chemotherapeutic Agent in PTK2 High Ovarian PDX Model of OV2423

In this experiment, a PTK2 high ovarian PDX model of OV2423 was established to evaluate the anti-tumor effect of Compound 5 in combination with chemotherapeutic agent carboplatin and/or paclitaxel (Harbin Pharmaceutical Group). The dosing regimen was as follows:
Compound 5: 50 mg/kg, orally, once per day, for a total of 28 days;
Carboplatin: 30 mg/kg, intraperitoneal injection, once a week, for a total of 28 days;
Paclitaxel: 10 mg/kg, intravenous injection, once a week, for a total of 28 days;

The dosing regimen of each drug in the dosing regimen for the combination is the same as the dosing regimen for the single drug.

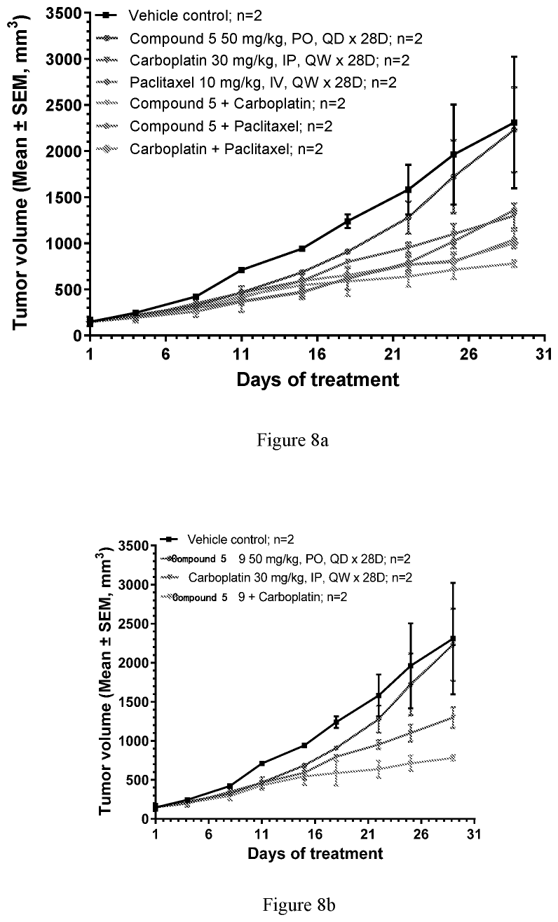

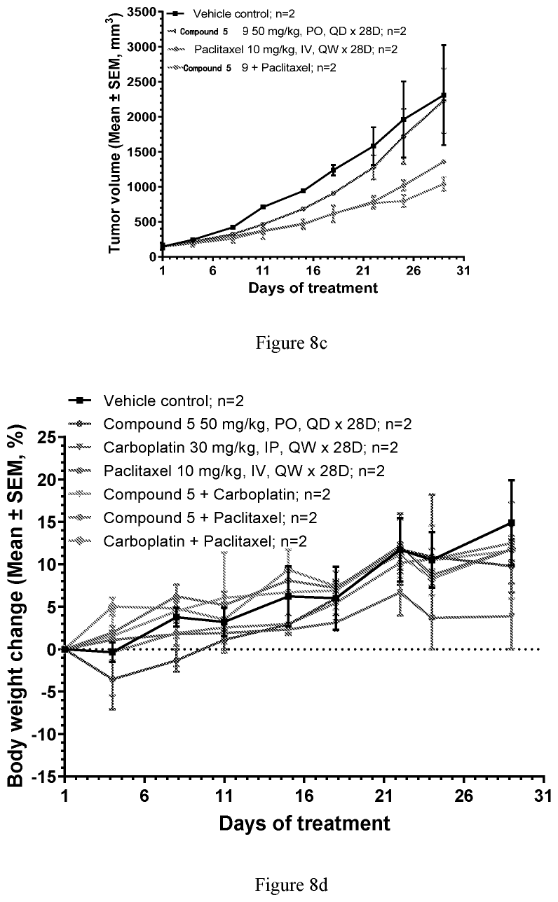

As shown in Table 7, FIGS. 8a and 8b, after 28 days of administration, the T/C (%) values of Compound 5, carboplatin and the combination of carboplatin with Compound 5 were 78.97%, 47.76% and 28.06% respectively, and the synergy factor was 1.34, indicating synergistic effects.

As shown in Table 7, FIGS. 8b and 8c, after 28 days of administration, the T/C (%) values of Compound 5, paclitaxel and the combination of paclitaxel with Compound 5 were 78.97%, 50.80% and 38.07% respectively, and the combination group had a synergy factor of 1.05, indicating synergistic effects.

In addition, as shown in FIG. 8d, little body weight loss (less than 5%) was observed in the combination groups during the study.

In conclusion, Compound 5 single agent showed minor antitumor activity, carboplatin single agent or paclitaxel single agent showed moderate antitumor activity, while combination treatment of Compound 5 and paclitaxel or combination treatment of Compound 5 and carboplatin achieved synergistic antitumor effects in s.c. ovarian cancer PDX model.

TABLE 7
Synergistic anti-tumor effect of Compound 5 in combination
with paclitaxel or Compound 5 in combination with carboplatin in
PTK2 high ovarian PDX OV2423 mouse xenograft tumor model

| | RTV on Day | T/C on Day | Synergistic | |
|---|---|---|---|---|
| | 28 after | 28 after | factor on | |
| | administration | admin- | Day 28 after | |
| | (mean ± | istration | admin- | |
| Treatment | standard error) | (%) | istration | mRECIST |
| Vehicle | 18.78 ± 10.48 | — | — | 2/2 PD |
| control | | | | |
| Compound 5 | 14.83 ± 3.19 | 78.97 | — | 2/2 PD |
| Carboplatin | 8.97 ± 1.24 | 47.76 | — | 2/2 PD |
| Paclitaxel | 9.54 ± 1.73 | 50.80 | — | 2/2 PD |
| Compound 5 + | 5.27 ± 0.72 | 28.06 | 1.34 | 2/2 PD |
| carboplatin | | | | |
| Compound 5 + | 7.15 ± 0.30 | 38.07 | 1.05 | 2/2 PD |
| paclitaxel | | | | |
| Carboplatin + | 6.51 ± 0.34 | 34.66 | 0.70 | 2/2 PD |
| paclitaxel | | | | |

n < 3, no statistical analysis applied

### Example 10. Combination Treatment with Compound 5 and Chemotherapeutic Agent in Ovarian PDX Model of OV1658

In this experiment, an ovarian PDX model of OV1658 was established to evaluate the anti-tumor effect of Compound 5 in combination with chemotherapeutic agent carboplatin and/or paclitaxel (Harbin Pharmaceutical Group). The dosing regimen was as follows:
Compound 5: 50 mg/kg, orally, once per day, for a total of 19 days;
Carboplatin: 30 mg/kg, intraperitoneal injection, once a week, for a total of 19 days;
Paclitaxel: 10 mg/kg, intravenous injection, once a week, for a total of 19 days;

The dosing regimen of each drug in the dosing regimen for the combination is the same as the dosing regimen for the single drug.

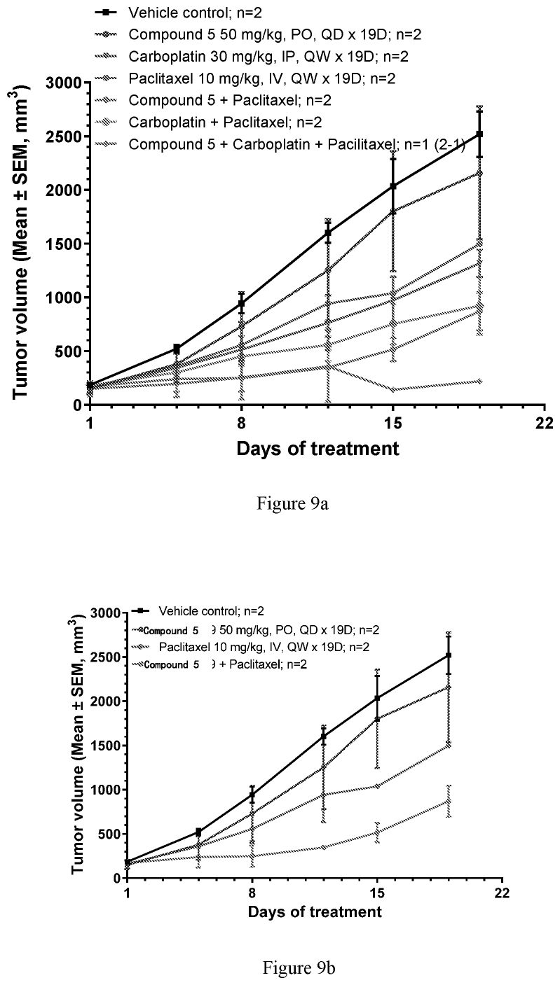

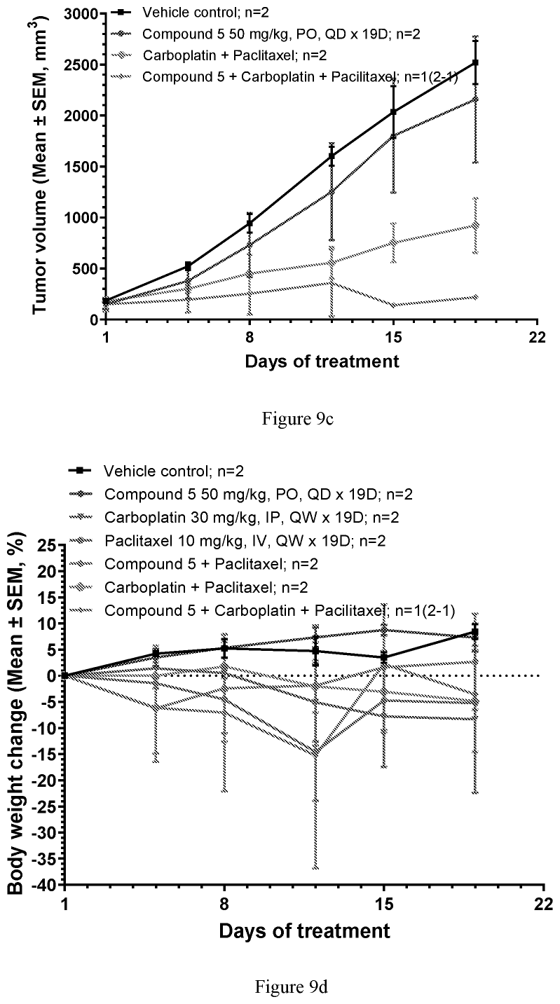

As shown in Table 8, FIGS. 9a and 9b, after 19 days of administration, the T/C (%) values of Compound 5, paclitaxel and the combination of paclitaxel with Compound 5 were 106.67%, 86.31% and 38.10% respectively, and the combination group had a synergistic factor of 2.40, indicating synergistic effects.

As shown in Table 8, FIGS. 9a and 9c, after 19 days of administration, the T/C (%) value of the combination of Compound 5 with paclitaxel and carboplatin was 15.81%, and the synergy factor was 2.52, indicating synergistic effects.

In conclusion, carboplatin single agent or paclitaxel single agent showed minor antitumor activity, while combination treatment of Compound 5 and paclitaxel or combination treatment of Compound 5 and paclitaxel plus carboplatin achieved synergistic antitumor effects in s.c. ovarian cancer PDX model.

TABLE 8
Synergistic anti-tumor effect of Compound 5 in combination
with paclitaxel or Compound 5 in combination with paclitaxel plus
carboplatin in ovarian PDX OV1658 mouse xenograft tumor model

| | RTV on Day 19 | | | | |
|---|---|---|---|---|---|
| | after | T/C on Day 19 | Synergistic | | mRECIST |
| | administration | after | factor on Day | | on Day |
| | (mean ± | administration | 19 after | | 19 after |
| Treatment | standard error) | (%) | administration | mRECIST | administration |
| Vehicle | 13.66 ± 0.96 | — | — | 2/2 PD | 2/2 PD |
| control | | | | | |
| Compound 5 | 14.57 ± 0.47 | 106.67 | — | 2/2 PD | 2/2 PD |
| Carboplatin | 9.34 ± 2.01 | 68.37 | — | 2/2 PD | 2/2 PD |
| Paclitaxel | 11.79 | 86.31 | — | 2/2 PD | 2/2 PD |
| Compound 5 + | 5.25 ± 1.44 | 38.10 | 2.40 | 2/2 PD | 2/2 PD |
| paclitaxel | | | | | |
| Carboplatin + | 5.10 ± 0.63 | 37.34 | 1.58 | 2/2 PD | 2/2 PD |
| paclitaxel | | | | | |
| Compound 5 + | 2.16 | 15.81 | 2.52 | 1/2 SD, | 2/2 PD |
| carboplatin + | | | | 1/2 PD | |
| paclitaxel | | | | | |

n < 3, no statistical analysis applied

### Example 11. Combination Treatment with Compound 5 and Chemotherapeutic Agent in PTK2 High Ovarian PDX Model of OV1385

In this experiment, a PTK2 high ovarian PDX model of OV1385 was established to evaluate the anti-tumor effect of Compound 5 in combination with chemotherapeutic agent carboplatin and/or paclitaxel (Harbin Pharmaceutical Group). The dosing regimen was as follows:
Compound 5: 50 mg/kg, orally, once per day, for a total of 35 days;
Carboplatin: 30 mg/kg, intraperitoneal injection, once a week, for a total of 35 days;
Paclitaxel: 10 mg/kg, intravenous injection, once a week, for a total of 35 days;

The dosing regimen of each drug in the dosing regimen for the combination is the same as the dosing regimen for the single drug.

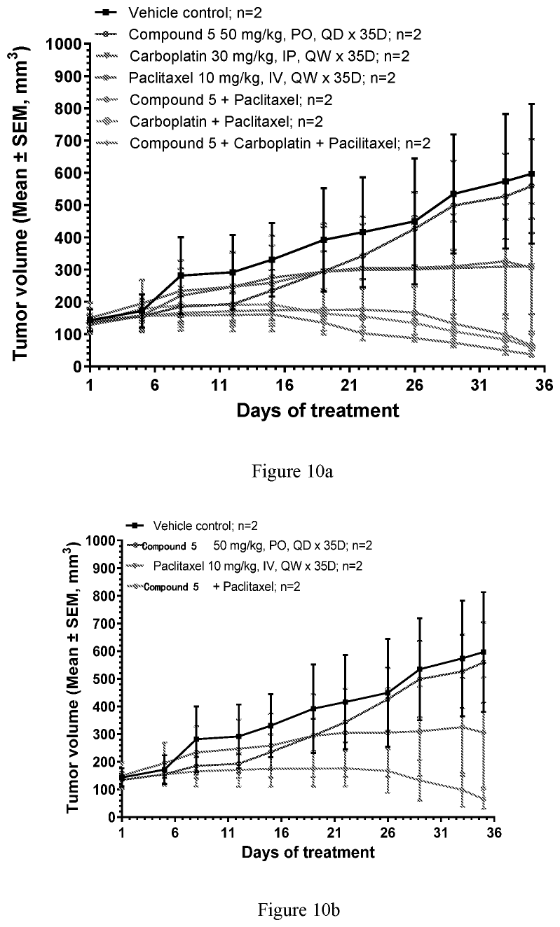

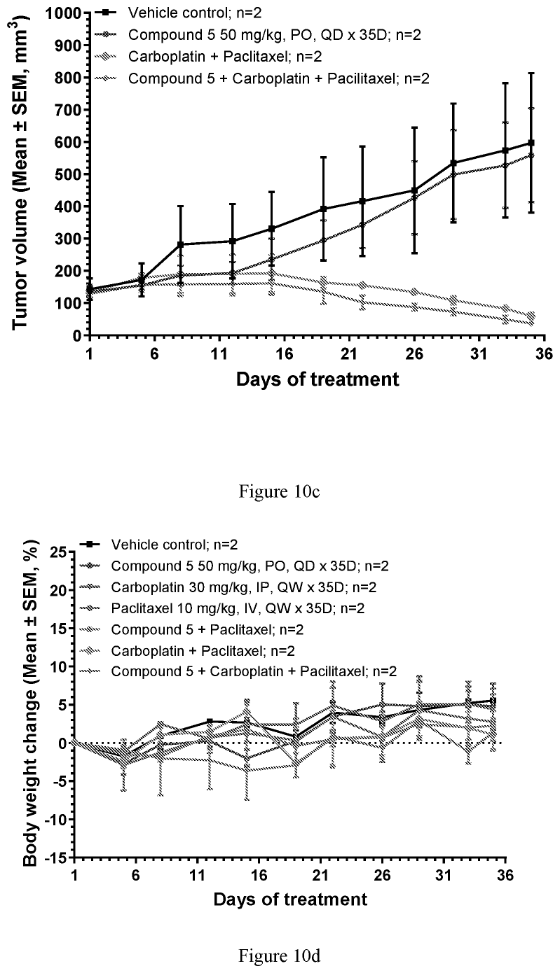

As shown in Table 9, FIGS. 10a and 10b, after 35 days of administration, the T/C (%) values of Compound 5, paclitaxel and the combination of paclitaxel with Compound 5 were 101.49%, 44.55% and 11.34% respectively, and the combination group had a synergistic factor of 3.97, indicating synergistic effects.

As shown in Table 9, FIGS. 10a and 10c, after 35 days of administration, the T/C (%) value of the combination of Compound 5 with paclitaxel and carboplatin was 7.18%, and the synergy factor was 1.57, indicating synergistic effects.

In addition, as shown in FIG. 10d, little body weight loss (less than 5%) was observed in the combination groups during the study.

In conclusion, carboplatin single agent or paclitaxel single agent showed moderate antitumor activity, while combination treatment of Compound 5 and paclitaxel or combination treatment of Compound 5 and paclitaxel plus carboplatin achieved synergistic antitumor effects in s.c. ovarian cancer PDX model.

TABLE 9
Synergistic anti-tumor effect of Compound 5 in combination
with paclitaxel or Compound 5 in combination with paclitaxel plus
carboplatin in PTK2 high ovarian PDX OV1385 mouse xenograft tumor model

| | RTV on Day 35 | | | | |
|---|---|---|---|---|---|
| | after | T/C on Day 35 | Synergistic | | mRECIST |
| | administration | after | factor on Day | | on Day |
| | (mean ± | administration | 35 after | | 35 after |
| Treatment | standard error) | (%) | administration | mRECIST | administration |
| Vehicle | 4.04 ± 0.56 | — | — | 2/2 PD | 2/2 PD |
| control | | | | | |
| Compound 5 | 4.10 ± 0.42 | 101.49 | — | 2/2 PD | 2/2 PD |
| Carboplatin | 2.08 ± 0.49 | 51.49 | — | 1/2 SD, | 2/2 PD |
| | | | | 1/2 PD | |
| Paclitaxel | 1.80 ± 0.75 | 44.55 | — | 2/2 PD | 2/2 PD |
| Compound 5 + | 0.46 ± 0.11 | 11.34 | 3.97 | 2/2 SD | 2/2 SD |
| paclitaxel | | | | | |
| Carboplatin + | 0.45 ± 0.09 | 11.14 | 2.06 | 1/2 SD, | 1/2 SD, |
| paclitaxel | | | | 1/2 PD | 1/2 PD |
| Compound 5 + | 0.29 ± 0.11 | 7.18 | 1.57 | 1/2 PR, | 1/2 PR, |
| carboplatin + | | | | 1/2 SD | 1/2 SD |
| paclitaxel | | | | | |

n < 3, no statistical analysis applied

### Example 12. Combination Treatment with Compound 5 and Chemotherapeutic Agent in PTK2 High Ovarian PDX Model of OV2018

In this experiment, a PTK2 high ovarian PDX model of OV2018 was established to evaluate the anti-tumor effect of Compound 5 in combination with chemotherapeutic agent paclitaxel (Harbin Pharmaceutical Group). The dosing regimen was as follows:
Compound 5: 50 mg/kg, orally, once per day, for a total of 35 days;
Paclitaxel: 10 mg/kg, intravenous injection, once a week, for a total of 35 days;

The dosing regimen of each drug in the dosing regimen for the combination is the same as the dosing regimen for the single drug.

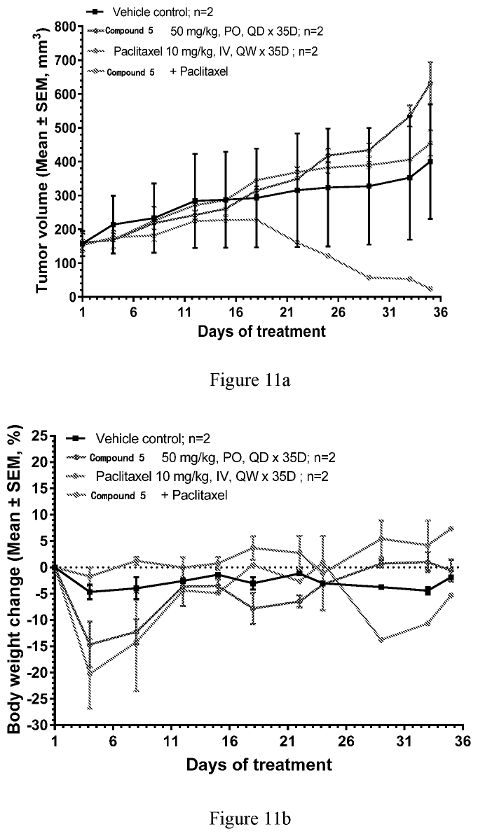

As shown in Table 10 and FIG. 11a, after 35 days of administration, the T/C (%) values of Compound 5, paclitaxel and the combination of paclitaxel with Compound 5 were 162.80%, 118.60% and 7.02% respectively, and the synergistic factor was 27.49, indicating strong synergistic effects.

In conclusion, Compound 5 singe agent and paclitaxel single agent showed no antitumor activity, while combination treatment of Compound 5 and paclitaxel achieved a synergistic antitumor effect in s.c. ovarian cancer PDX model.

TABLE 10
Synergistic anti-tumor effect of Compound 5 in
combination with paclitaxel in PTK2 high ovarian PDX model OV2018
mouse xenograft tumor model

| | RTV on Day 35 | | | | |
|---|---|---|---|---|---|
| | after | T/C on Day 35 | Synergistic | | mRECIST |
| | administration | after | factor on Day | | on Day |
| | (mean ± | administration | 35 after | | 35 after |
| Treatment | standard error) | (%) | administration | mRECIST | administration |
| Vehicle | 2.42 ± 0.51 | — | — | 1/2 SD, | 2/2 PD |
| control | | | | 1/2 PD | |
| Compound 5 | 3.94 ± 0.41 | 162.80 | — | 2/2 PD | 2/2 PD |
| Paclitaxel | 2.87 ± 0.24 | 118.60 | — | 2/2 PD | 2/2 PD |
| Compound 5 + | 0.17 | 7.02 | 27.49 | 1/2 SD | 1/2 SD |
| paclitaxel | | | | | |

n < 3, no statistical analysis applied

### Example 13. Compound 5 Combined with Paclitaxel Exert Synergistic Antitumor Activity in OVCAR3 s.c. Ovarian Xenograft Model

In this experiment, an OVCAR3 subcutaneous ovarian xenograft model was established to evaluate the anti-tumor effect of Compound 5 in combination with chemotherapeutic agent paclitaxel and carboplatin (Selleck). The dosing regimen was as follows:
Compound 5: 100 mg/kg, orally, once per day, for a total of 35 days;
Paclitaxel: 10 mg/kg, intravenous injection, once a week, for a total of 48 days;
Carboplatin: 30 mg/kg, intravenous injection, once a week, for a total of 57 days;

The dosing regimen of each drug in the dosing regimen for the combination is the same as the dosing regimen for the single drug.

The cell source is China Center for Type Culture Collection (CCTCC), and the cell culture is RPMI 1640 medium with 300 mg/L (2 mM) L-glutamine adjusted to contain 1.5 g/L sodium bicarbonate, 4.5 g/L glucose, 10 mM HEPES, and 1.0 mM sodium pyruvate, 80%; fetal bovine serum, 20%; P/S 1%.

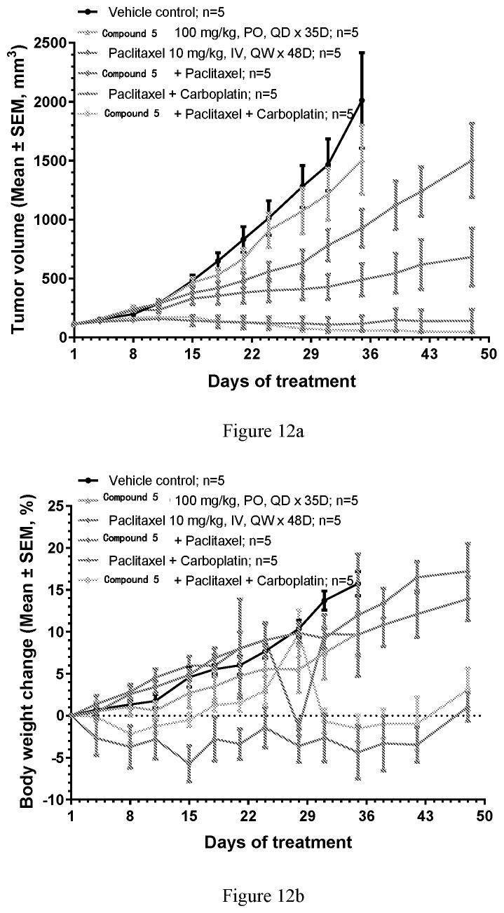

As shown in Table 11 and FIG. 12a, after 35 days of administration, the T/C (%) values of Compound 5, paclitaxel and the combination of paclitaxel and Compound 5 were 85.04%, 52.14% and 4.99% respectively, and the synergistic factor was 7.06, indicating strong synergistic effects; the T/C (%) values of Compound 5 combined with paclitaxel plus carboplatin was 2.89%, and the synergistic factor was 7.08, indicating synergistic effects. Animals from both the combination group of Compound 5 and paclitaxel or the combination group of Compound 5 and paclitaxel plus carboplatin achieved ⅖ PR, ORR=40%.

In addition, as shown in FIG. 12b, little body weight loss (less than 5%) was observed in the combination group of Compound 5 and paclitaxel or the combination group of Compound 5 and paclitaxel plus carboplatin during the study, indicating acceptable toxicity.

In conclusion, Compound 5 single agent showed minor antitumor activity, and paclitaxel single agent showed moderate antitumor activity, while the combination treatment of Compound 5 and paclitaxel or the combination treatment of Compound 5 and paclitaxel plus carboplatin achieved synergistic antitumor effects in s.c. ovarian cancer PDX model.

TABLE 11
Synergistic anti-tumor effect of Compound 5 in
combination with paclitaxel or Compound 5 in combination with
paclitaxel plus carboplatin in OVCAR3 s.c. ovarian mouse xenograft
tumor model

| | RTV on Day 35 | T/C on Day 35 | Synergistic | | Disease | Overall |
|---|---|---|---|---|---|---|
| | after administration | after | factor on Day | | control | regression |
| | (mean ± | administration | 35 after | | rate | rate |
| Treatment | standard error) | (%) | administration | mRECIST | (DCR), % | (ORR), % |
| Vehicle | 17.63 ± 3.14 | — | — | 5/5 PD | 0 | 0 |
| control | | | | | | |
| Compound 5 | 13.66 ± 2.88 | 85.04 | — | 5/5 PD | 0 | 0 |
| Paclitaxel | 8.02 ± 1.30 | 52.14 | — | 5/5 PD | 0 | 0 |
| Compound 5 + paclitaxel | 0.88 ± 0.38*§ | 4.99 | 7.06 | 2/5 PR, SD, 1/5 PD | 2/580% | 40% |
| Paclitaxel + | 4.66 ± 1.63 | 26.41 | — | 5/5 PD | 0 | 0 |
| carboplatin | | | | | | |
| Compound 5 + | 0.51 ± 0.16*§ | 2.89 | 7.08 | 2/5 PR, 3/5 SD | 100% | 40% |
| paclitaxel + | | | | | | |
| carboplatin | | | | | | |

*p < 0.05, compared to vehicle control group,
§p < 0.05, compared to paclitaxel group

### Example 14. Compound 5 May Overcome Resistance to Paclitaxel or Resistance to Paclitaxel and Carboplatin in OVCAR3 s.c. Ovarian Xenograft Model

In this experiment, an OVCAR3 subcutaneous ovarian xenograft model was established to evaluate the activity of Compound 5 in overcoming the resistance to chemotherapeutic agent paclitaxel and carboplatin (Selleck). The dosing regimen was as follows:

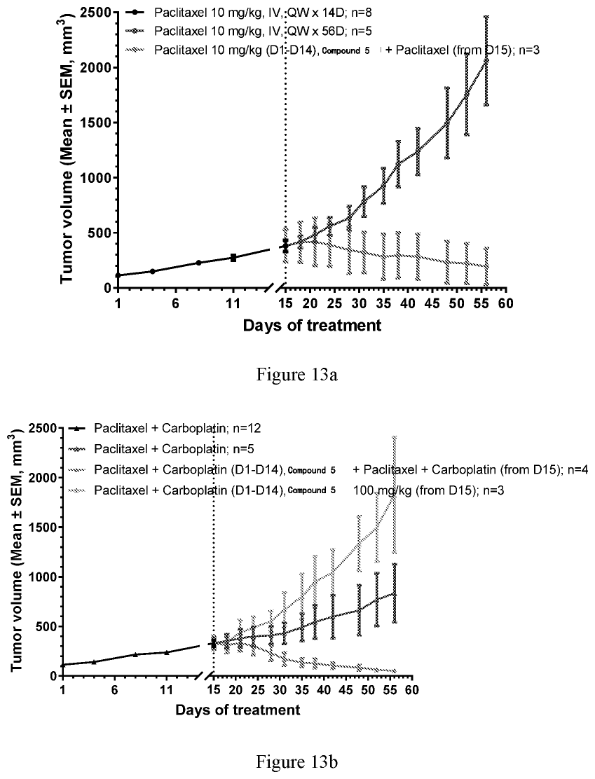

Paclitaxel (10 mg/kg) was intravenously injected to the mice once a week for the first 14 days (n=8). Randomize the mice into two groups on Day 15, one group (n=5) was continuously intravenously injected for paclitaxel single agent (10 mg/kg, once a week) until Day 56, and the other group (n=3) was treated with the combination of paclitaxel (10 mg/kg, once a week) and Compound 5 (100 mg/kg, orally, once per day) from Day 15 until Day 56. The results are shown in Table 12 and FIG. 13a.

Paclitaxel (10 mg/kg, intravenous injection, once a week) and carboplatin (30 mg/kg, intravenous injection, once a week) combination treatment was used for the mice for the first 14 days (n=12). Randomize the mice into three groups on Day 15, one group (n=5) continuously underwent the same amount of paclitaxel and carboplatin combination treatment until Day 56, another group (n=4) underwent paclitaxel plus carboplatin and Compound 5 (100 mg/kg, orally, once per day) combination treatment, and the third group (n=3) underwent Compound 5 single agent treatment. The results are shown in Table 12 and FIG. 13b.

The cell source and cell culture are the same with Example 13.

As shown in Table 12 and FIG. 13a, paclitaxel and compound 5 combination treatment showed enhanced antitumor activity compared with paclitaxel single agent treatment group, and achieved ⅔ PR, ORR=66.67% compared to 0 in paclitaxel single agent group.

As shown in Table 12 and FIG. 13b, paclitaxel plus carboplatin and Compound 5 combination treatment showed enhanced antitumor activity compared with paclitaxel and carboplatin combination treatment or Compound 5 single agent treatment group, and achieved 4/4 PR, ORR=100% compared to 0 in the other groups.

TABLE 12
Compound 5 may overcome resistance to chemotherapeutic
agents in OVCAR3 s.c. ovarian mouse xenograft tumor model

| | RTV on Day | | | |
|---|---|---|---|---|
| | 56 after | | | |
| | administration | | | |
| | (mean ± | | | |
| | standard | | DCR | ORR |
| Treatment | error) | mRECIST | (%) | (%) |
| Paclitaxel | 5.19 ± 0.62 | 1/5 SD, | 20 | 0 |
| | | 4/5 PD | | |
| Paclitaxel (D1-D14), | 0.34 ± 0.22 | 2/3 PR, | 100 | 66.67 |
| Compound 5 + | | 1/3 SD | | |
| paclitaxel (from D15) | | | | |
| Paclitaxel + carboplatin | 2.40 ± 0.50 | 4/5 SD, | 80 | 0 |
| | | 1/5 PD | | |
| Paclitaxel + carboplatin | 0.15 ± 0.03 | 4/4 PR | 100 | 100 |
| (D1-D14), Compound 5 + | | | | |
| paclitaxel + | | | | |
| carboplatin (from D15) | | | | |
| Paclitaxel + carboplatin | 5.32 ± 0.67 | 3/3 PD | 0 | 0 |

(D1-D14), Compound 5
from D15)

### Example 15. Combination treatment with Compound 5 and Compound 33 in s.c. LUAD A549 (KRAS, STK11, ATR mut) Zenograft Model

In this experiment, a s.c. LUAD A549 (KRAS, STK11, ATR mut) xenograft model was established to evaluate the anti-tumor effect of Compound 5 in combination with Compound 33. The dosing regimen was as follows:
Compound 5: 100 mg/kg, orally, once per day, for a total of 53 days;
Compound 33: 50 mg/kg, orally, once per day, from D1 to D14, D22 to D36 and from D44 to D53;

The dosing regimen of each drug in the dosing regimen for the combination is the same as the dosing regimen for the single drug.

The cell source is Cobioer, and the cell culture is RPMI 1640 medium with 300 mg/L (2 mM) L-glutamine adjusted to contain 2.0 g/L sodium bicarbonate, 90%; fetal bovine serum, 10%; P/S 1%.

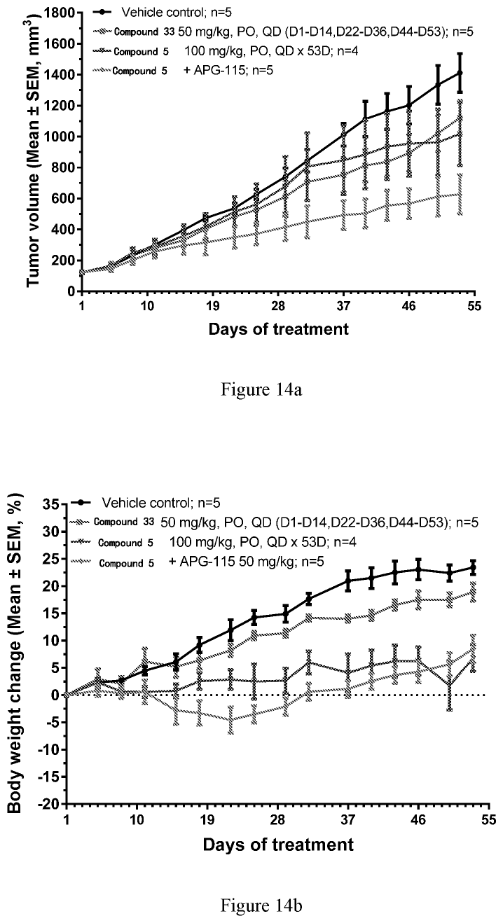

As shown in Table 13 and FIG. 14a, after 53 days of administration, the T/C (%) values of Compound 5, Compound 33 and the combination of Compound 33 with Compound 5 were 78.91%, 71.66% and 43.85% respectively, and the synergistic factor was 1.29, indicating synergistic effects.

In addition, as shown in FIG. 14b, little body weight loss (less than 5%) was observed in the combination group of Compound 5 and Compound 33 during the study, indicating acceptable toxicity.

In conclusion, Compound 5 single agent and Compound 33 single agent showed minor antitumor activity, while combination treatment of Compound 5 and Compound 33 achieved a synergistic antitumor effect in s.c. A549 NSCLC xenograft model.

TABLE 13
Synergistic anti-tumor effect of Compound 5 in combination
with Compound 33 in A549 NSCLC xenograft tumor model

| | RTV on Day 53 | | |
|---|---|---|---|
| | after | T/C on Day 53 | Synergistic |
| | administration | after | factor on Day |
| | (mean ± | administration | 53 after |
| Treatment | standard error) | (%) | administration |
| Vehicle | 11.75 ± 1.32 | — | — |
| control | | | |
| Compound 5 | 8.42 ± 1.63 | 78.91 | — |
| Compound 33 | 9.27 ± 0.63 | 71.66 | — |
| Compound 5 + | 5.15 ± 0.88** | 43.85 | 1.29 |
| Compound 33 | | | |

**p < 0.01 compared to vehicle control group

### Example 16. Combination Treatment with Compound 5 and Compound 33 in s.c. Neuroblastoma SH-SY5Y (ALK F1174L) Xenograft Model

In this experiment, a s.c. Neuroblastoma SH-SY5Y (ALK F1174L) xenograft model was established to evaluate the anti-tumor effect of Compound 5 in combination with Compound 33. The dosing regimen was as follows:
Compound 5: 50 mg/kg, orally, once per day, for a total of 21 days;
Compound 33: 50 mg/kg, orally, once per day, from D1 to D10, and orally administrated once on D21;

The dosing regimen of each drug in the dosing regimen for the combination is the same as the dosing regimen for the single drug.

The cell source is ATCC, and the cell culture is F12K: MEM=1:1 with sodium pyruvate (1×) and NEAA (1×), 90%; fetal bovine serum, 10%; P/S, 1%.

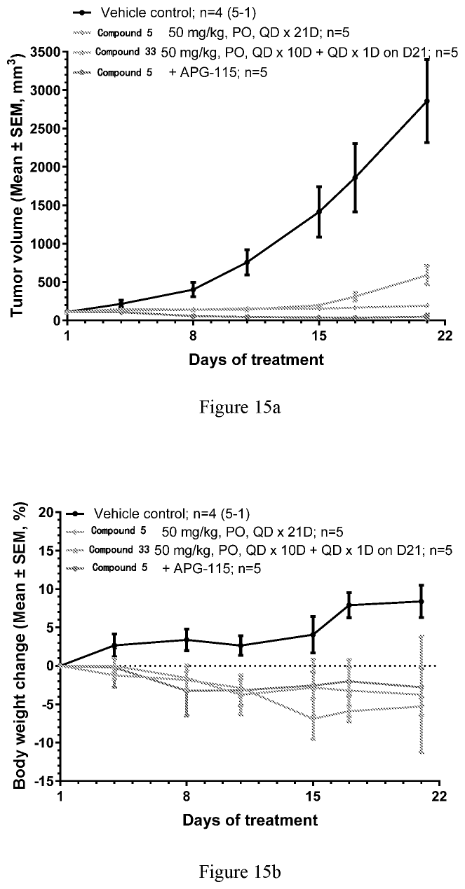

As shown in Table 14 and FIG. 15a, after 21 days of administration, the T/C (%) values of Compound 5, Compound 33 and the combination of Compound 33 with Compound 5 were 6.74%, 19.38% and 1.49% respectively. Animals from the combination group achieved ⅖ CR, ⅖ PR, ORR=80%.

In addition, as shown in FIG. 15b, little body weight loss (less than 10%) was observed in the combination group of Compound 5 and Compound 33 during the study, indicating acceptable toxicity.

In conclusion, Compound 5 single agent and Compound 33 single agent showed strong antitumor activity, and combination treatment of Compound 5 and Compound 33 achieved an enhanced antitumor effect in s.c. SH-SYSY Neuroblastoma xenograft model, achieved ORR 80% compared to 0 in other groups.

TABLE 14
Synergistic anti-tumor effect of Compound 5 in
combination with Compound 33 in Neuroblastoma SH-SY5Y xenograft
tumor model

| | RTV on Day | T/C on | Synergistic | | | | |
|---|---|---|---|---|---|---|---|
| | 21 after | Day 21 | factor | | | | |
| | administrati | after | on Day | | | | |
| | on (mean + | admini- | 21 after | | mRECIST on | | |
| | standard | stration | administ- | | Day 21 after | | ORR |
| Treatment | error) | (%) | ration | mRECIST | administration | DCR | (%) |
| Vehicle | 26.51 ± 4.39 | — | — | 5/5 PD | 5/5 PD | 0 | 0 |
| control | | | | | | | |
| Compound | 1.79 ± 0.22 | 6.74 | — | 3/5 SD, 2/52/5 PD | SD, 3/560 PD | 60 | 0 |
| 5 | | | | | | | |
| Compound | 5.14 ± 0.95 | 19.38 | — | 3/5 SD, 2/55/5 PD | PD | 60 | 0 |
| 33 | | | | | | | |
| Compound | 0.39 ± 0.25* | 1.49 | Around 1 | PR, 1/5 SD 2/5 | PR, 1/5 SD CR, 2/5 | 100 | 80 |
| 5 + | | | | CR, 2/52/5 | | | |
| Compound | | | | | | | |
| 33 | | | | | | | |

*p < 0.05, compared to Compound 5 single agent group

### Example 17. Combination Treatment with Compound 5 and Panobinostat in s.c. A549 NSCLC Lung Cancer

In this experiment, a subcutaneous A549 lung cancer model was established to evaluate the anti-tumor effect of Compound 5 in combination with HDAC inhibitor panobinostat. The dosing regimen was as follows:
Compound 5: 100 mg/kg, orally, once per day, for a total of 14 days;
Panobinostat: 3 mg/kg, intraperitoneal injection, administrated for 5 days then take a break for 2 days, for a total of 14 days;

The dosing regimen of each drug in the dosing regimen for the combination is the same as the dosing regimen for the single drug.

The cell source is Cobioer, and the cell culture is RPMI 1640 medium with 300 mg/L (2 mM) L-glutamine adjusted to contain 2.0 g/L sodium bicarbonate, 90%; fetal bovine serum, 10%; P/S 1%.

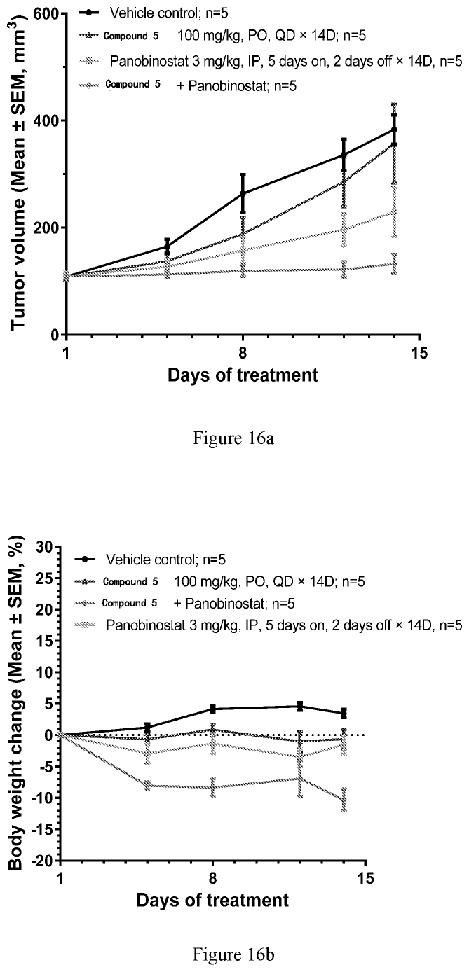

As shown in Table 15 and FIG. 16a, after 14 days of administration, the T/C (%) values of Compound 5, panobinostat and the combination of panobinostat with Compound 5 were 89.54%, 57.54% and 33.79% respectively, and the combination group had a synergy factor of 1.82, indicating synergistic effects.

In conclusion, Compound 5 single agent showed minor antitumor activity, panobinostat single agent showed moderate antitumor activity, while combination treatment of Compound 5 and panobinostat achieved a synergistic antitumor effect in s.c. A549 NSCLC xenograft model.

TABLE 15
Synergistic anti-tumor effect of Compound 5 in combination
with panobinostat in A549 NSCLC xenograft tumor model

| | RTV on Day | T/C on | Synergistic | |
|---|---|---|---|---|
| | 14 after | Day 14 | factor on | |
| | administration | after | Day 14 | |
| | (mean ± | admin- | after | |
| | standard | istration | admin- | |
| Treatment | error) | (%) | istration | mRECIST |
| Vehicle | 3.58 ± 0.31 | — | — | |
| control | | | | |
| Compound 5 | 3.21 ± 0.46 | 89.54 | — | |
| panobinostat | 2.06 ± 0.29 | 57.54 | — | |
| Compound 5 + | 1.21 ± 0.14** | 33.79 | 1.82 | 4/5 SD, |
| anobinostat | | | | 1/5 PD |

**p < 0.01 compared to vehicle control group

### Example 18. Combination Treatment with Compound 5 and Anti-PD-1 Antibody in s.c. Syngeneic Tumor Model of Colon CT26

In this experiment, a s.c. syngeneic tumor model of colon CT26 was established to evaluate the anti-tumor effect of Compound 5 in combination with anti-PD-1 antibody (Bioxcell, Item No. BE0146, Clone No: RMP1-14). The dosing regimen was as follows:
Compound 5: 30 mg/kg, orally, once per day, for a total of 12 days;
Compound 5: 100 mg/kg, orally, once per day, for a total of 12 days;
Anti-PD-1 antibody: 10 mg/kg, intraperitoneal injection, twice weekly, for a total of 12 days;

The dosing regimen of each drug in the dosing regimen for the combination is the same as the dosing regimen for the single drug.

The cell source is Cobioer, and the cell culture is RPMI 1640+10% FBS+1% P/S.

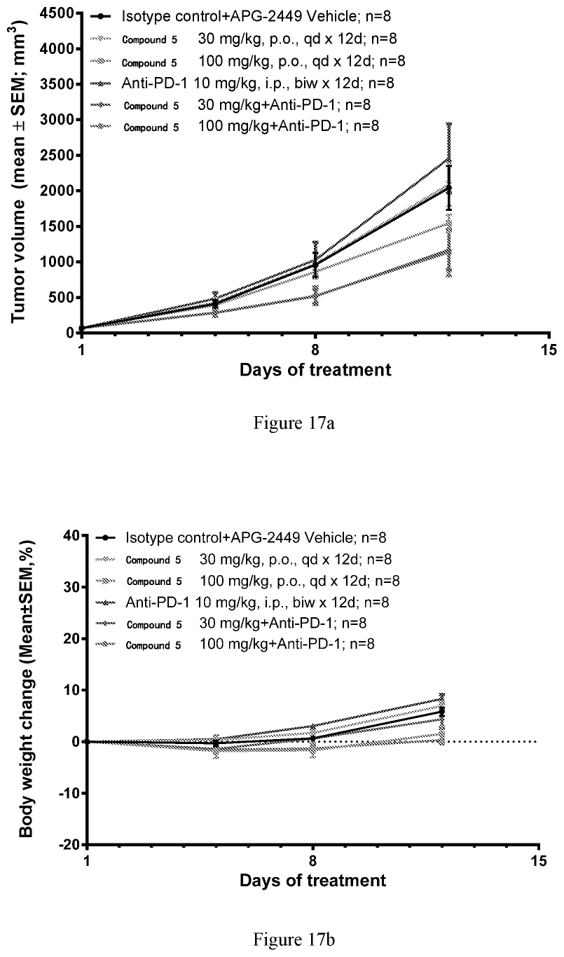

As shown in Table 16 and FIG. 17a, on Day 12, the T/C (%) values of the combination of anti-PD-1 antibody with 30 mg/kg Compound 5 and the combination of anti-PD-1 antibody with 100 mg/kg Compound 5 were 58.76% and 56.36% respectively, and the synergistic factors were 2.10 and 1.64 respectively, indicating synergistic effects.

In addition, as shown in FIG. 17b, little body weight loss (less than 5%) was observed in the combination group of Compound 5 and anti-PD-1 during the study, indicating acceptable toxicity.

In conclusion, Compound 5 single agent or anti-PD-1 single agent showed no antitumor activity, while the combination treatments achieved enhanced antitumor activities and synergistic antitumor effect in s.c. CT26 syngeneic colon cancer tumor model.

TABLE 16
Synergistic anti-tumor effect of Compound 5 in combination
with anti-PD-1 antibody in syngeneic tumor model of colon
CT26

| | RTV on Day | | |
|---|---|---|---|
| | 12 after | | |
| | administration | T/C on Day 12 | |
| | (mean ± | after | Synergistic factor |
| | standard | administration | on Day 12 after |
| Treatment | error) | (%) | administration |
| Vehicle control | 29.1 ± 4.0 | — | — |
| Compound 5, 30 | 29.9 ± 4.2 | 102.75 | — |
| mg/kg | | | |
| Compound 5, 100 | 22.5 ± 2.2 | 77.32 | — |
| mg/kg | | | |
| Anti-PD-1 | 34.9 ± 6.4 | 119.93 | |
| Anti-PD-1 + | 17.1 ± 4.2 | 58.76 | 2.10 |
| Compound 5, 30 | | | |
| mg/kg | | | |
| Anti-PD-1 + | 16.4 ± 4.9 | 56.36 | 1.64 |
| Compound 5, 100 | | | |
| mg/kg | | | |

### Example 19. Combination Treatment with Compound 5 and Olaparib in s.c. FAK-high NSCLC LU-01-0751 PDX (BRCA2 mut)

In this experiment, a s.c. FAK-high NSCLC LU-01-0751 PDX (BRCA2 mut) model (Wuxi Pharma Tech) was established to evaluate the anti-tumor effect of Compound 5 in combination with PARP inhibitor Olaparib (Selleck). The dosing regimen was as follows:
Compound 5: 50 mg/kg, orally, once per day, for a total of 21 days, (dosing holiday is from D7 to D13);
Olaparib: 50 mg/kg, orally, twice a day, for a total of 21 days;

The dosing regimen of each drug in the dosing regimen for the combination is the same as the dosing regimen for the single drug.

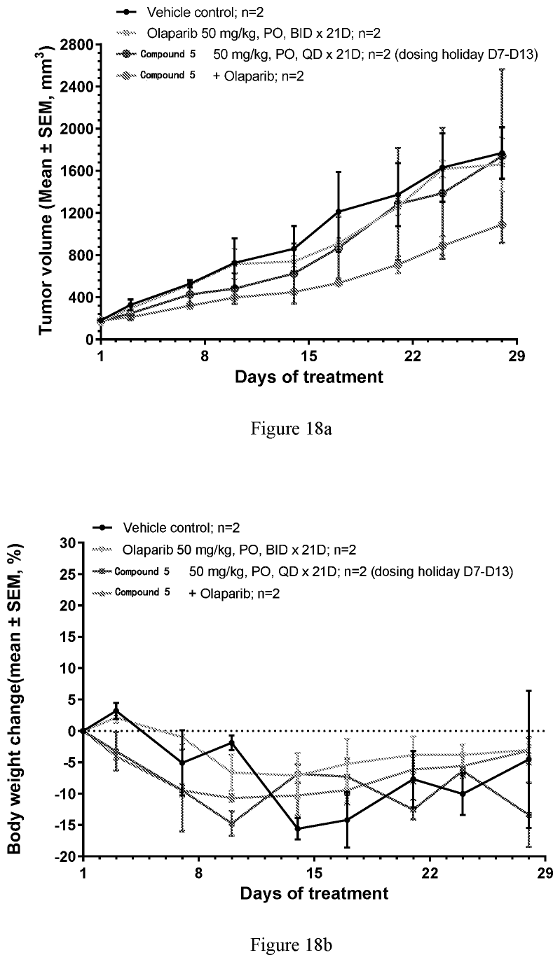

As shown in Table 17 and FIG. 18a, on Day 28, the T/C (%) values of Compound 5, Olaparib and the combination of Olaparib with Compound 5 were 104.83%, 96.37% and 62.25% respectively, and the synergistic factor was 1.62, indicating synergistic effects.

In conclusion, Compound 5 single agent or Olaparib single agent showed no antitumor activity, while the combination treatment with Compound 5 and Olaparib achieved a synergistic antitumor effect in s.c. NSCLC PDX model.

TABLE 17
Synergistic anti-tumor effect of Compound 5 in combination with
Olaparib in NSCLC LU-01-0751 PDX xenograft tumor
model

| | RTV on Day | T/C on Day | Synergistic | |
|---|---|---|---|---|
| | 28 after | 28 after | factor on | |
| | administration | admin- | Day 28 after | |
| | (mean ± | istration | admin- | |
| Treatment | standard error) | (%) | istration | mRECIST |
| Vehicle | 9.88 ± 1.87 | — | — | 2/2 PD |
| control | | | | |
| Compound 5 | 10.38 ± 3.46 | 104.83 | — | 2/2 PD |
| Olaparib | 9.51 ± 3.78 | 96.37 | — | 2/2 PD |
| Compound 5 + | 6.15 ± 0.67 | 62.25 | 1.62 | 2/2 PD |
| Olaparib | | | | |

### Example 20. Combination Treatment with Compound 5 and Lenvatinib in PTK2-High Liver PDX Model LI-03-1140

In this experiment, a PTK2-high liver PDX model of LI-03-1140 (Wuxi Pharma Tech) was established to evaluate the anti-tumor effect of Compound 5 in combination with VEGF inhibitor Lenvatinib (Selleck). The dosing regimen was as follows:
Compound 5: 50 mg/kg, orally, once per day, for a total of 28 days;
Lenvatinib: 20 mg/kg, orally, once per day, for a total of 28 days;

The dosing regimen of each drug in the dosing regimen for the combination is the same as the dosing regimen for the single drug.

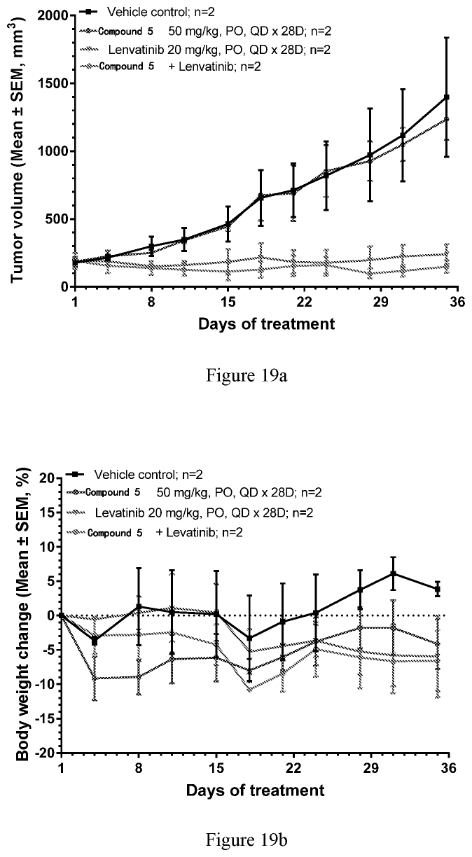

As shown in Table 18 and FIG. 19a, on Day 28, the T/C (%) values of Compound 5, Lenvatinib and the combination of Lenvatinib with Compound 5 were 92.08%, 23.40% and 9.21% respectively, and the synergistic factor was 2.33, indicating synergistic effects.

In conclusion, Compound 5 single agent showed no antitumor activity, Lenvatinib single agent showed strong antitumor activity, and the combination treatment achieved a synergistic antitumor effect in s.c. liver cancer PDX model.

TABLE 18
Synergistic anti-tumor effect of Compound 5 in
combination with Lenvatinib in PTK2-high liver PDX model of LI-03-1140

| | RTV on Day | | | | |
|---|---|---|---|---|---|
| | 28 after | | | | |
| | administration | T/C on Day 28 | Synergistic | | |
| | (mean ± | after | factor on Day | | mRECIST on |
| | standard | administration | 28 after | | Day 28 after |
| Treatment | error) | (%) | administration | mRECIST | administration |
| Vehicle | 5.43 ± 1.78 | — | — | 2/2 PD | 2/2 PD |
| control | | | | | |
| Compound | 5.00 ± 0.17 | 92.08 | — | 2/2 PD | 2/2 PD |
| 5 | | | | | |
| Lenvatinib | 1.27 ± 0.88 | 23.40 | — | 1/2 PR, | 1/2 PR, |
| | | | | 1/2 PD | 1/2 PD |
| Compound | 0.50 ± 0.03 | 9.21 | 2.33 | 1/2 PR, | 2/2 SD |
| 5 + | | | | 1/2 SD | |
| Lenvatinib | | | | | |

n < 3, no statistical analysis applied

### Example 21 Combination Treatment with Compound 33 or Compound 5 and BRAFi+MEKi in TP53^{wt,} BRAF^{v600E,} NRAS^{wt}, PTEN^{wt}, CDKN2A^{mut} Cutaneous Melanoma Model

In this experiment, a TP53wt , BRAFv600E , NRASwt, PTENwt, CDKN2Amut C32 cutaneous melanoma model (Wuxi Pharma Tech) was established to evaluate the anti-tumor effect of Compound 5 in combination with BRAFi (Dabrafenib)+MEKi (trametinib) (Selleck). The dosing regimen was as follows:
Compound 5: 50 mg/kg, PO QD 21 D, for a total of 25 days;
Dabrafenib +trametinib: Dabrafenib 30mg/kg,QD QD 21 D for a total of 25 days, trametinib 1 mg/kg

The dosing regimen of each drug in the dosing regimen for the combination is the same as the dosing regimen for the single drug.

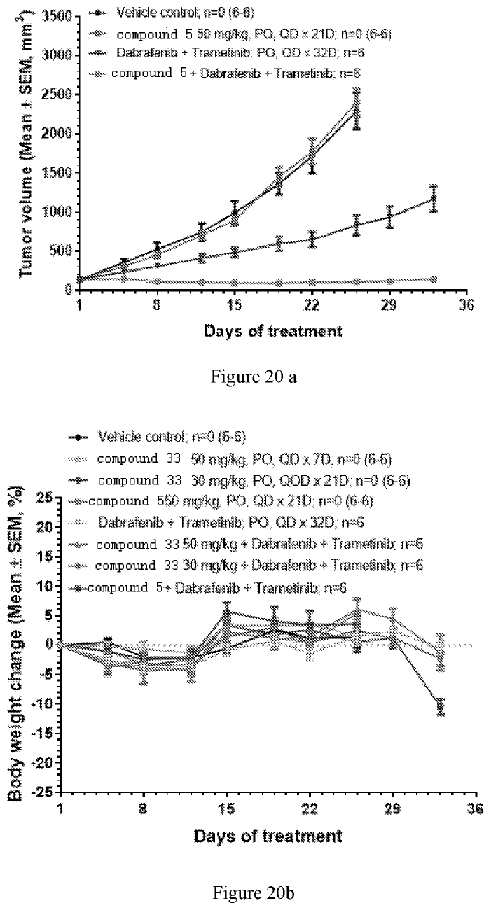

As shown in Table19 and FIG. 20, on Day 25, the T/C (%) values of Compound 5, Dabrafenib+trametinib, and the combination of Dabrafenib+trametinib with Compound 5 were 106.09%, 35.39% and 4.55% respectively, and the synergistic factor was 8.25, indicating synergistic effects.

TABLE 19

| | RTV % @ | T/C % @ | | Synergy@ |
|---|---|---|---|---|
| Group | D25 | D25 | mRECIST | D25 |
| Vehicle control | — | — | — | — |
| COMPOUND 5 | 17.80 ± 1.3 | 106.09 | — | — |
| 50 mg/kg | | | | |
| Dabrafenib + | 5.94 ± | 35.39 | 6/6 PD | |
| Trametinib | 0.64** | | | |
| COMPOUND 5 + | 0.76 ± | 4.55 | 2/6 PR, | 8.25 |
| Dabrafenib + | 0.08***§§\|\| | | 4/6 SD | |
| Trametinib | | | | |

DCR: Disease control rate, DCR is calculated with the proportion of animals demonstrating CR, PR, or SD based on mRECIST; ORR: Objective response rate, ORR is calculated with the proportion of animals demonstrating CR or PR based on mRECIST.

In conclusion, Compound 5 single agent showed no antitumor activity, Dabrafenib +trametinib showed strong antitumor activity, and the combination treatment achieved a synergistic antitumor effect in TP53^{wt,} BRAF^{v600E,} NRAS^{wt}, PTEN^{wt}, CDKN2A^{mut} C32 cutaneous melanoma model.

### Example 22 Combination Treatment with COMPOUND 5 and Palbociclib/Trametinib/TMZ in U-87-MG Subcutaneous Glioblastoma

In this experiment, a U-87-MG subcutaneous glioblastoma model (Wuxi Pharma Tech) was established to evaluate the anti-tumor effect of Compound 5 in combination with Palbociclib/Trametinib/TMZ. The dosing regimen was as follows:
Compound 5: 100 mg/kg, PO QD 21 D;
Palbociclib: 20mg/kg,QD QD 21 D,
Trametinib: 0.5 mg/kg QD QD 21 D,
TMZ 50 mg/kg QD QD 21 D,

The dosing regimen of each drug in the dosing regimen for the combination is the same as the dosing regimen for the single drug.

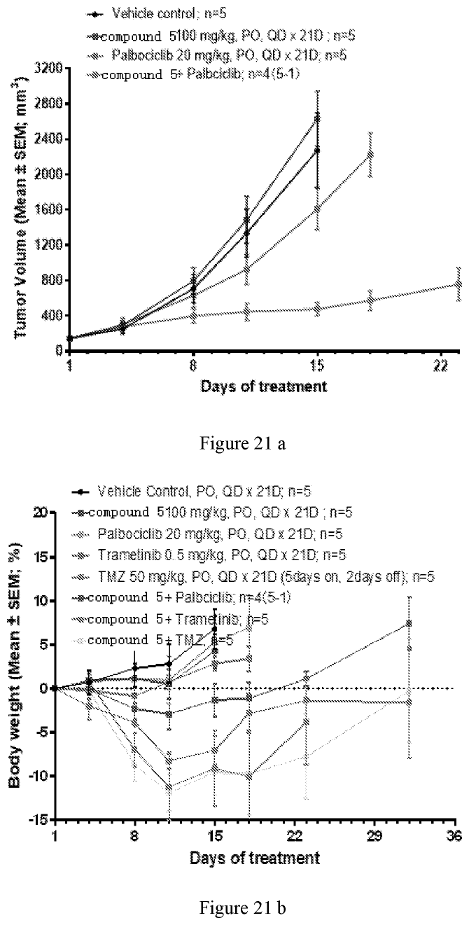

As shown in Table 20 and FIG. 21, on Day 15, the T/C (%) values of Compound 5, Palbociclib , and the combination of Palbociclib with Compound 5 were 120.7%, 72.6% and 22.0% respectively, and the synergistic factor was 3.988, indicating synergistic effects.

TABLE 20

| | | T/C | | |
|---|---|---|---|---|
| | | (%) | Synergy | |
| | | @ | ratio | |
| Group | RTV @ D15 | D15 | @ D15 | mRECIST |
| Vehicle Control | 15.0 ± 1.3 | — | — | 5/5 PD |
| COMPOUND 5, | 18.1 ± 1.1 | 120.7 | — | 5/5 PD |
| 100 mg/kg, QD | | | | |
| Palbociclib, 20 | 10.9 ± 0.6 | 72.6 | — | 5/5 PD |
| mg/kg, QD | | | | |
| COMPOUND 5 + | 3.3 ± 0.5***^{###&&&} | 22.0 | 3.988 | 5/5 PD |
| Palbociclib | | | | |

*P < 0.05, vs. vehicle control;
**P < 0.01, vs. vehicle control;
***P < 0.001, vs. vehicle control;
^{###}P < 0.001, vs. COMPOUND 5;
^{&&&}P < 0.001, vs. Palbociclib;
^{$}P < 0.05, vs. Trametinib;
Synergy: Ratio > 1, synergistic; Ratio = 1, additive; Ratio < 1, antagonistic.

In conclusion, Compound 5 single agent showed no antitumor activity, and the combination treatment achieved a synergistic antitumor effect in U-87-MG subcutaneous glioblastoma model.

ABBREVIATIONS AND SPECIALIST TERMS

| | |
|---|---|
| Mean ± SD | Mean ± standard deviation |
| Mean ± SEM | Mean ± Standard error mean |
| MDSC | Myeloid-derived suppressor cell |
| mg/kg | Milligram per kilogram |
| MSD | Meso Scale Discovery |
| n | Number |
| PK | Pharmacokinetic/Pharmacokinetics |
| PO or p.o. | Oral administration |
| i.p. | Intraperitoneal injection |
| i.v. | Intravenous injection |
| PBS | Phosphate buffer saline |
| PR | Partial (tumor) regression (i.e., tumor volumes become |
| | smaller compared to before treatment) |
| CR | Complete regression |
| SD | Stable disease |
| PD | Progression disease |
| DCR | Disease control rate, which is calculated with the |
| | proportion of animals demonstrating CR, PR and SD |
| ORR | Overall response rate, which is calculated with the |
| | proportion of animals demonstrating CR and PR |
| PD-1 | programmed death 1 |
| PD-L1 | programmed death-ligand 1 |
| qd | Once a day |
| q2d | Once every two days |
| QOD | Every other day |
| QW | Once a week |
| BIW | Twice weekly |
| BID | Twice a day |
| Response rate | % of responsive animals in each treatment group, |
| | including CR, PR and SD |
| RTV | Relative Tumor Volume (RTV = Vₜ/V₁; V₁ and Vₜ are |
| | the average tumor volumes on the first day of treatment |
| | (day 1) and the average tumor volumes on a certain |
| | time point (day T) |
| SD | Stable disease |
| SEM | Standard error of mean |
| SPF | Specific-pathogen-free |
| SPSS | Statistical Product and Service Solutions |
| Synergy | Synergy score = ((A/C) [symbol] (B/C))/(AB/C); A = |
| score/ratio/ | response to treatment A; B = response to treatment B; |
| factor | C = response to vehicle control; AB = combination of |
| | treatment A and B. |
| T/C (%) | T/C (%) = (T_{RTV}/C_{RTV}) [symbol] 100%; T_{RTV} is RTV of the |
| | treatment group and C_{RTV} is RTV of the control group. |
| TIL | Tumor infiltrating lymphocyte |
| TV | Tumor volume |
| W, WK or wk | Week |

What is claimed is:

1. A pharmaceutical composition comprising an ALK inhibitor, and one or more anticancer reagents selected from a CDK4/6 inhibitor, an Mek inhibitor, a BRAF inhibitor, a chemotherapeutic agent, an MDM2 inhibitor, an HDAC inhibitor, a PD-1 inhibitor, a PARP inhibitor, a VEGF inhibitor and a BCR-ABL inhibitor.

2. The pharmaceutical composition according to claim 1, wherein the ALK inhibitor is a compound of Formula I or a pharmaceutically acceptable salt or solvate thereof: 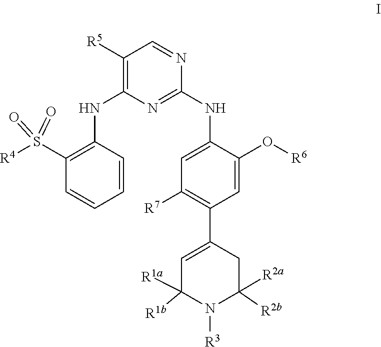
wherein:
R^{1a} and R^{1b} are independently selected from the group consisting of hydrogen, C₁₋₆ alkyl, and C₃₋₈ cycloalkyl;
R^{1a} and R^{2b} are independently selected from the group consisting of hydrogen, C₁₋₆ alkyl, and C₃₋₈ cycloalkyl;
R³ is selected from the group consisting of hydrogen, C₁₋₆ alkyl, C₃₋₆ cycloalkyl, and 4- to 8-membered heterocyclyl,
R⁴ is selected from the group consisting of C₁₋₄ alkyl and C₃₋₆ cycloalkyl;
R⁵ is halo;
R⁶ is selected from the group consisting of C₁₋₄ alkyl and C₃₋₆ cycloalkyl; and
R⁷ is selected from the group consisting of hydrogen, C₁₋₄ alkyl, and C₃₋₆ cycloalkyl,
with proviso that when R^{1a}, R^{1b}, R^{2a}, and R^{2b} are each hydrogen, then R³ is selected from the group consisting of C₃₋₆ cycloalkyl and 4- to 8-membered heterocyclyl.

3. The pharmaceutical composition according claim 1, wherein the ALK inhibitor is a compound of any one of Formula (II) to Formula (VI) or a pharmaceutically acceptable salt or solvate thereof: 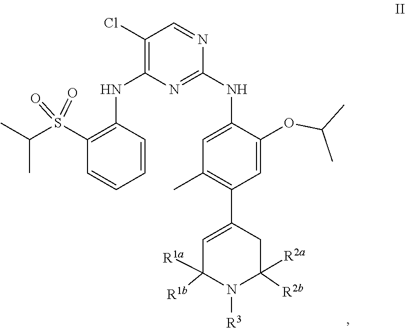
wherein:
R^{1a} and R^{1b} are independently selected from the group consisting of hydrogen, C₁₋₄ alkyl, and C₃₋₆ cycloalkyl;
R^{2a} and R^{2b} are independently selected from the group consisting of hydrogen, C₁₋₄ alkyl, and C₃₋₆ cycloalkyl; and
R³ is selected from the group consisting of hydrogen, C₁₋₄ alkyl, C₃₋₆ cycloalkyl, and 4- to 8-membered heterocyclyl. or 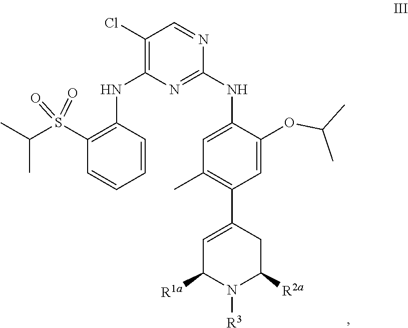
wherein:
R^{1a} and R^{2a} are each independently selected from the group consisting of C₁₋₄ alkyl, and C₃₋₆ cycloalkyl; and
the compound has an enantiomeric excess of about 90% or more; or 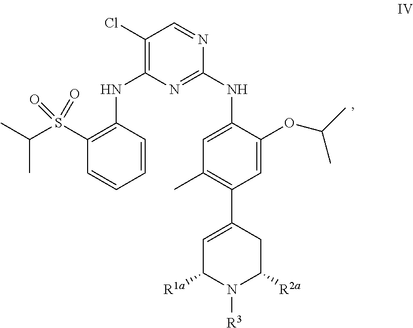
wherein:
R^{1a} and R^{2a} are each independently selected from the group consisting of C₁₋₄ alkyl, and C₃₋₆ cycloalkyl; and
the compound has an enantiomeric excess of about 90% or more., or 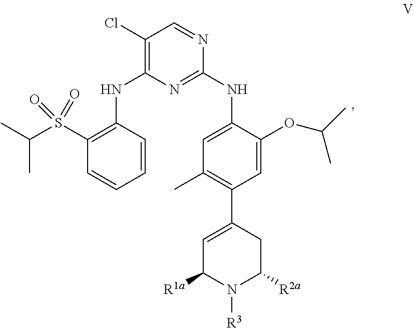
wherein:
R^{1a} and R^{2a} are each independently selected from the group consisting of C₁₋₄ alkyl, and C₃₋₆ cycloalkyl; and
the compound has an enantiomeric excess of about 90% or more; or 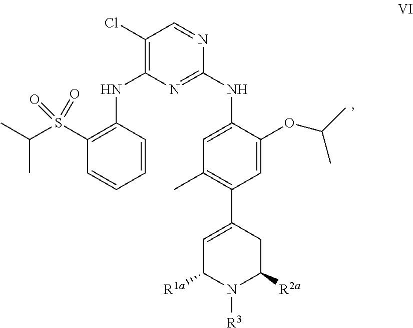
wherein:
R^{1a} and R^{2a} are each independently selected from the group consisting of C₁₋₄ alkyl, and C₃₋₆ cycloalkyl; and
the compound has an enantiomeric excess of about 90% or more.

4-7. (canceled)

8. The pharmaceutical composition according to claim 1, wherein the ALK inhibitor is a compound in the following table or a pharmaceutically acceptable salt or solvate thereof:

| No. | Structure | Name |
|---|---|---|
| 1 | 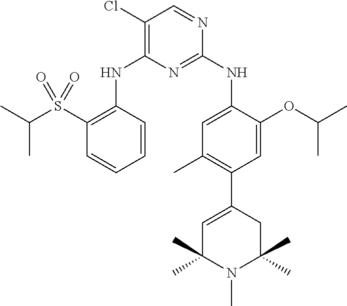 | 5-chloro-N²-(2-isopropoxy-5-methyl-4-(1,2,2,6,6- pentamethyl-1,2,3,6-tetrahydropyridin-4- yl)phenyl)-N⁴-(2- (isopropylsulfonyl)phenyl)pyrimidine-2,4- diamine; |
| 2 | 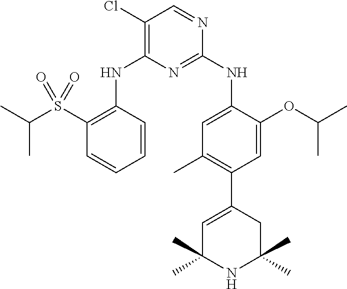 | 5-chloro-N²-(2-isopropoxy-5-methyl-4-(2,2,6,6- tetramethyl-1,2,3,6-tetrahydropyridin-4- yl)phenyl)-N⁴-(2- (isopropylsulfonyl)phenyl)pyrimidine-2,4- diamine; |
| 3 | 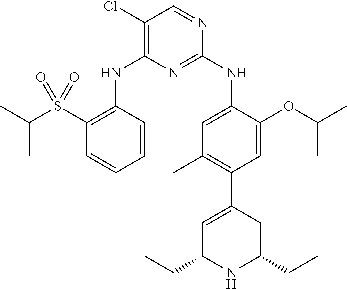 [SMILES: CC[C@H]1CC(c2cc(OC(C)C)c(Nc3ncc(Cl)c(Nc4ccccc4S(=O)(=O)C(C)C)n3)cc2C)=C[C@@H](CC)N1] | 5-chloro-N²-(4-((cis)-2,6-diethyl-1,2,3,6- tetrahydropyridin-4-yl)-2-isopropoxy-5- methylphenyl)-N⁴-(2- (isopropylsulfonyl)phenyl)pyrimidine-2,4- diamine; |
| 4 | 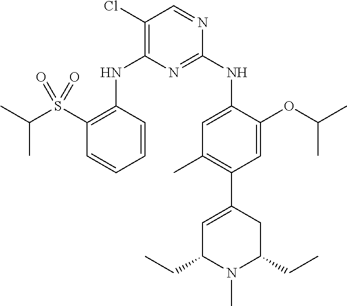 [SMILES: CC[C@H]1CC(c2cc(OC(C)C)c(Nc3ncc(Cl)c(Nc4ccccc4S(=O)(=O)C(C)C)n3)cc2C)=C[C@@H](CC)N1C] | 5-chloro-N²-(4-((cis)-2,6-diethyl-1-methyl-1,2,3,6- tetrahydropyridin-4-yl)-2-isopropoxy-5- methylphenyl)-N⁴-(2- (isopropylsulfonyl)phenyl)pyrimidine-2,4- diamine; |
| 5 | 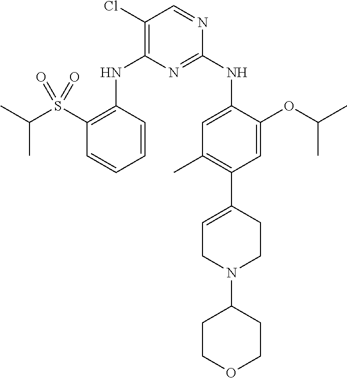 [SMILES: Cc1cc(Nc2ncc(Cl)c(Nc3ccccc3S(=O)(=O)C(C)C)n2)c(OC(C)C)cc1C1=CCN(C2CCOCC2)CC1] | 5-chloro-N²-(2-isopropoxy-5-methyl-4-(1- (tetrahydro-2H-pyran-4-yl)-1,2,3,6- tetrahydropyridin-4-yl)phenyl)-N⁴-(2- (isopropylsulfonyl)phenyl)pyrimidine-2,4- diamine; |
| 6 | 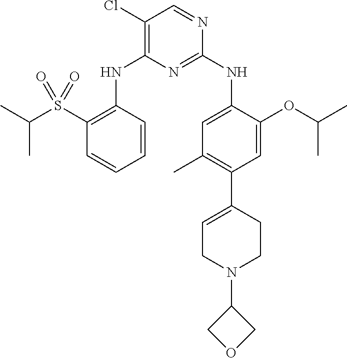 [SMILES: Cc1cc(Nc2ncc(Cl)c(Nc3ccccc3S(=O)(=O)C(C)C)n2)c(OC(C)C)cc1C1=CCN(C2COC2)CC1] | 5-chloro-N²-(2-isopropoxy-5-methyl-4-(1-(oxetan- 3-yl)-1,2,3,6-tetrahydropyridin-4-yl)phenyl)-N⁴- (2-(isopropylsulfonyl)phenyl)pyrimidine-2,4- diamine; |
| 7 | 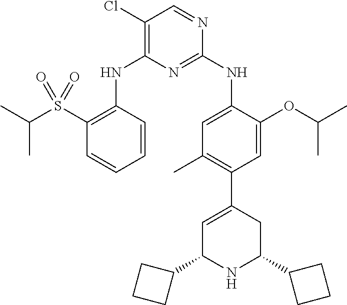 [SMILES: Cc1cc(Nc2ncc(Cl)c(Nc3ccccc3S(=O)(=O)C(C)C)n2)c(OC(C)C)cc1C1=C[C@@H](C2CCC2)N[C@@H](C2CCC2)C1] | 5-chloro-N²-(4-((cis)-2,6-dicyclobutyl-1,2,3,6- tetrahydropyridin-4-yl)-2-isopropoxy-5- methylphenyl)-N⁴-(2- (isopropylsulfonyl)phenyl)pyrimidine-2,4- diamine; |
| 8 | 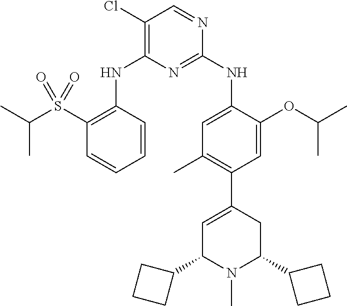 [SMILES: Cc1cc(Nc2ncc(Cl)c(Nc3ccccc3S(=O)(=O)C(C)C)n2)c(OC(C)C)cc1C1=C[C@@H](C2CCC2)N(C)[C@@H](C2CCC2)C1] | 5-chloro-N²-(4-((cis)-2,6-dicyclobutyl-1-methyl- 1,2,3,6-tetrahydropyridin-4-yl)-2-isopropoxy-5- methylphenyl)-N⁴-(2- (isopropylsulfonyl)phenyl)pyrimidine-2,4- diamine; |
| 9 | 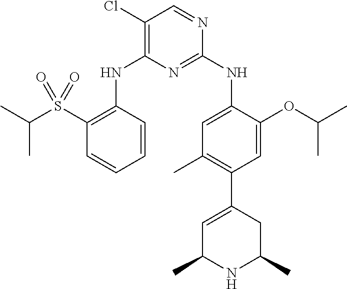 [SMILES: Cc1cc(Nc2ncc(Cl)c(Nc3ccccc3S(=O)(=O)C(C)C)n2)c(OC(C)C)cc1C1=C[C@H](C)N[C@H](C)C1] | 5-chloro-N²-(4-((cis)-2,6-dimethyl-1,2,3,6- tetrahydropyridin-4-yl)-2-isopropoxy-5- methylphenyl)-N⁴-(2- (isopropylsulfonyl)phenyl)pyrimidine-2,4- diamine; |
| 10 | 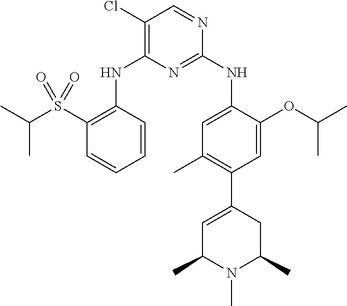 [SMILES: Cc1cc(Nc2ncc(Cl)c(Nc3ccccc3S(=O)(=O)C(C)C)n2)c(OC(C)C)cc1C1=C[C@H](C)N(C)[C@H](C)C1] | 5-chloro-N²-(2-isopropoxy-5-methyl-4-((cis)- 1,2,6-trimethyl-1,2,3,6-tetrahydropyridin-4- yl)phenyl)-N⁴-(2- (isopropylsulfonyl)phenyl)pyrimidine-2,4- diamine; |
| 11 | 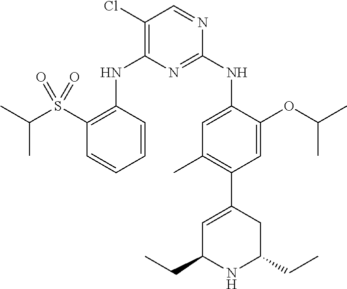 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1] | 5-chloro-N²-(4-((trans)-2,6-diethyl-1,2,3,6- tetrahydropyridin-4-yl)-2-isopropoxy-5- methylphenyl)-N⁴-(2- (isopropylsulfonyl)phenyl)pyrimidine-2,4- diamine; |
| 12 | 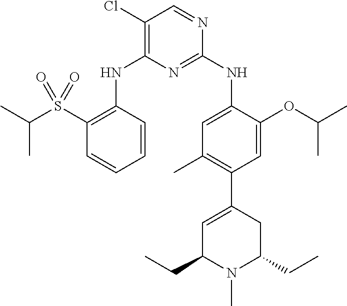 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1C] | 5-chloro-N²-(4-((trans)-2,6-diethyl-1-methyl- 1,2,3,6-tetrahydropyridin-4-yl)-2-isopropoxy-5- methylphenyl)-N⁴-(2- (isopropylsulfonyl)phenyl)pyrimidine-2,4- diamine; |
| 13 | 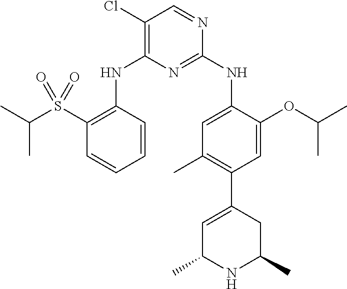 [SMILES: Cc1cc(Nc2ncc(Cl)c(Nc3ccccc3S(=O)(=O)C(C)C)n2)c(OC(C)C)cc1C1=C[C@@H](C)N[C@H](C)C1] | 5-chloro-N²-(4-((trans)-2,6-dimethyl-1,2,3,6- tetrahydropyridin-4-yl)-2-isopropoxy-5- methylphenyl)-N⁴-(2- (isopropylsulfonyl)phenyl)pyrimidine-2,4- diamine; |
| 14 | 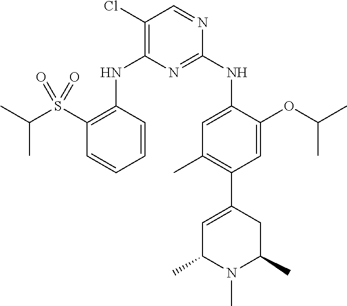 [SMILES: Cc1cc(Nc2ncc(Cl)c(Nc3ccccc3S(=O)(=O)C(C)C)n2)c(OC(C)C)cc1C1=C[C@@H](C)N(C)[C@H](C)C1] | 5-chloro-N²-(2-isopropoxy-5-methyl-4-((trans)- 1,2,6-trimethyl-1,2,3,6-tetrahydropyridin-4- yl)phenyl)-N⁴-(2- (isopropylsulfonyl)phenyl)pyrimidine-2,4- diamine; |
| 15 | 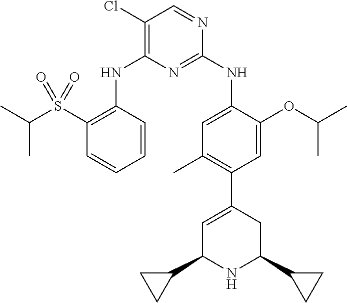 [SMILES: Cc1cc(Nc2ncc(Cl)c(Nc3ccccc3S(=O)(=O)C(C)C)n2)c(OC(C)C)cc1C1=C[C@H](C2CC2)N[C@H](C2CC2)C1] | 5-chloro-N²-(4-((cis)-2,6-dicyclopropyl-1,2,3,6- tetrahydropyridin-4-yl)-2-isopropoxy-5- methylphenyl)-N⁴-(2- (isopropylsulfonyl)phenyl)pyrimidine-2,4- diamine; |
| 16 | 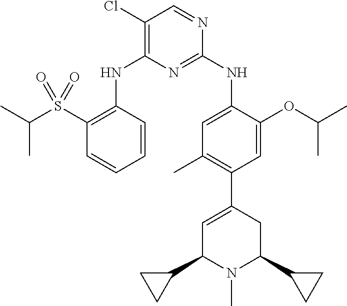 [SMILES: Cc1cc(Nc2ncc(Cl)c(Nc3ccccc3S(=O)(=O)C(C)C)n2)c(OC(C)C)cc1C1=C[C@H](C2CC2)N(C)[C@H](C2CC2)C1] | 5-chloro-N²-(4-((cis)-2,6-dicyclopropyl-1-methyl- 1,2,3,6-tetrahydropyridin-4-yl)-2-isopropoxy-5- methylphenyl)-N⁴-(2- (isopropylsulfonyl)phenyl)pyrimidine-2,4- diamine; |
| 17 | 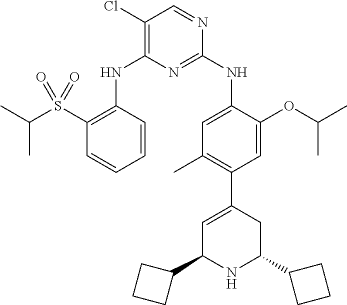 [SMILES: Cc1cc(Nc2ncc(Cl)c(Nc3ccccc3S(=O)(=O)C(C)C)n2)c(OC(C)C)cc1C1=C[C@H](C2CCC2)N[C@@H](C2CCC2)C1] | 5-chloro-N²-(4-((trans)-2,6-dicyclobutyl-1,2,3,6- tetrahydropyridin-4-yl)-2-isopropoxy-5- methylphenyl)-N⁴-(2- (isopropylsulfonyl)phenyl)pyrimidine-2,4- diamine; |
| 18 | 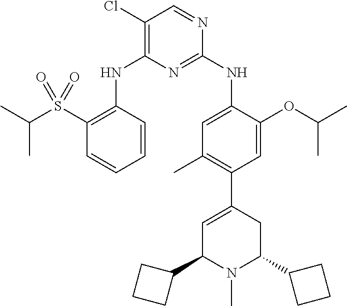 [SMILES: Cc1cc(Nc2ncc(Cl)c(Nc3ccccc3S(=O)(=O)C(C)C)n2)c(OC(C)C)cc1C1=C[C@H](C2CCC2)N(C)[C@@H](C2CCC2)C1] | 5-chloro-N²-(4-((trans)-2,6-dicyclobutyl-1-methyl- 1,2,3,6-tetrahydropyridin-4-yl)-2-isopropoxy-5- methylphenyl)-N⁴-(2- (isopropylsulfonyl)phenyl)pyrimidine-2,4- diamine; |
| 19 | 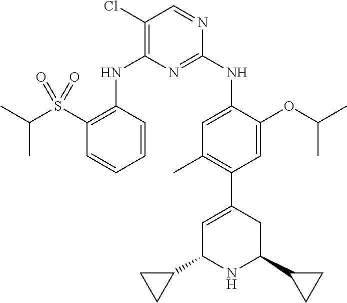 [SMILES: Cc1cc(Nc2ncc(Cl)c(Nc3ccccc3S(=O)(=O)C(C)C)n2)c(OC(C)C)cc1C1=C[C@@H](C2CC2)N[C@H](C2CC2)C1] | 5-chloro-N²-(4-((trans)-2,6-dicyclopropyl-1,2,3,6- tetrahydropyridin-4-yl)-2-isopropoxy-5- methylphenyl)-N⁴-(2- (isopropylsulfonyl)phenyl)pyrimidine-2,4- diamine; |
| 20 | 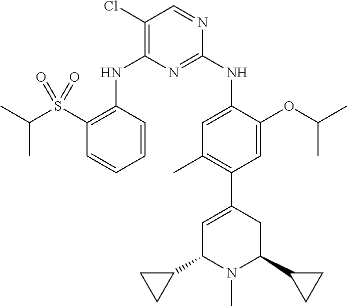 [SMILES: Cc1cc(Nc2ncc(Cl)c(Nc3ccccc3S(=O)(=O)C(C)C)n2)c(OC(C)C)cc1C1=C[C@@H](C2CC2)N(C)[C@H](C2CC2)C1] | 5-chloro-N²-(4-((trans)-2,6-dicyclopropyl-1- methyl-1,2,3,6-tetrahydropyridin-4-yl)-2- isopropoxy-5-methylphenyl)-N⁴-(2- (isopropylsulfonyl)phenyl)pyrimidine-2,4- diamine; |
| 21 | 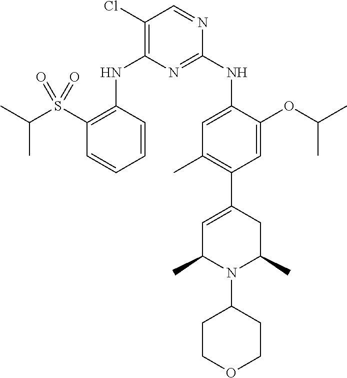 [SMILES: Cc1cc(Nc2ncc(Cl)c(Nc3ccccc3S(=O)(=O)C(C)C)n2)c(OC(C)C)cc1C1=C[C@H](C)N(C2CCOCC2)[C@H](C)C1] | 5-chloro-N²-(4-((cis)-2,6-dimethyl-1-(tetrahydro- 2H-pyran-4-yl)-1,2,3,6-tetrahydropyridin-4-yl)-2- isopropoxy-5-methylphenyl)-N⁴-(2- (isopropylsulfonyl)phenyl)pyrimidine-2,4- diamine; |
| 22 | 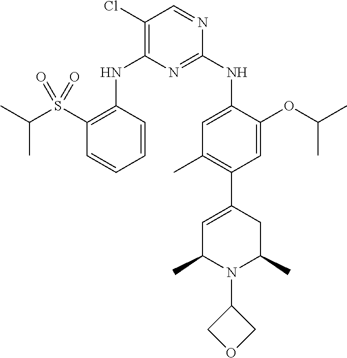 [SMILES: Cc1cc(Nc2ncc(Cl)c(Nc3ccccc3S(=O)(=O)C(C)C)n2)c(OC(C)C)cc1C1=C[C@H](C)N(C2COC2)[C@H](C)C1] | 5-chloro-N²-(4-((cis)-2,6-dimethyl-1-(oxetan-3- yl)-1,2,3,6-tetrahydropyridin-4-yl)-2-isopropoxy- 5-methylphenyl)-N⁴-(2- (isopropylsulfonyl)phenyl)pyrimidine-2,4- diamine; |
| 23 | 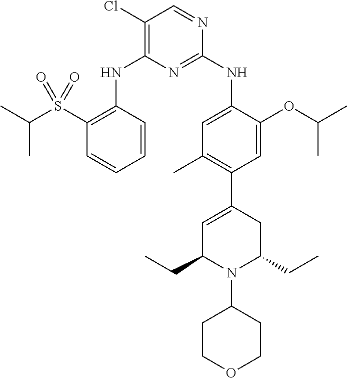 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1C1CCOCC1] | 5-chloro-N²-(4-((trans)-2,6-diethyl-1-(tetrahydro- 2H-pyran-4-yl)-1,2,3,6-tetrahydropyridin-4-yl)-2- isopropoxy-5-methylphenyl)-N⁴-(2- (isopropylsulfonyl)phenyl)pyrimidine-2,4- diamine; |
| 24 | 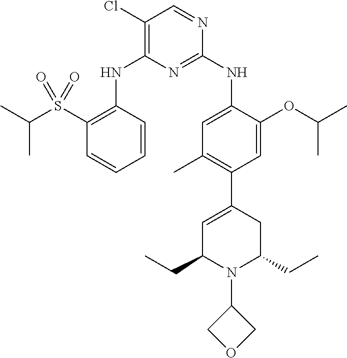 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1C1COC1] | 5-chloro-N²-(4-((2S,6S)-2,6-diethyl-1-(oxetan-3- yl)-1,2,3,6-tetrahydropyridin-4-yl)-2-isopropoxy- 5-methylphenyl)-N⁴-(2- (isopropylsulfonyl)phenyl)pyrimidine-2,4- diamine; |
| 25 | 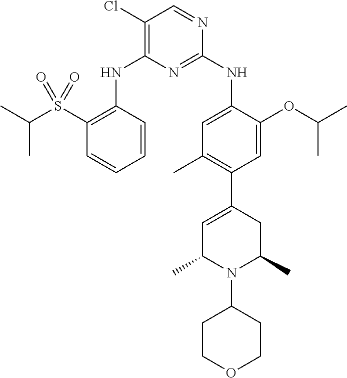 [SMILES: Cc1cc(Nc2ncc(Cl)c(Nc3ccccc3S(=O)(=O)C(C)C)n2)c(OC(C)C)cc1C1=C[C@@H](C)N(C2CCOCC2)[C@H](C)C1] | 5-chloro-N²-(4-((trans)-2,6-dimethyl-1- (tetrahydro-2H-pyran-4-yl)-1,2,3,6- tetrahydropyridin-4-yl)-2-isopropoxy-5- methylphenyl)-N⁴-(2- (isopropylsulfonyl)phenyl)pyrimidine-2,4- diamine; |
| 26 | 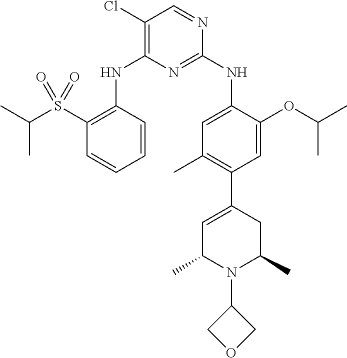 [SMILES: Cc1cc(Nc2ncc(Cl)c(Nc3ccccc3S(=O)(=O)C(C)C)n2)c(OC(C)C)cc1C1=C[C@@H](C)N(C2COC2)[C@H](C)C1] | 5-chloro-N²-(4-((trans)-2,6-dimethyl-1-(oxetan-3- yl)-1,2,3,6-tetrahydropyridin-4-yl)-2-isopropoxy- 5-methylphenyl)-N⁴-(2- (isopropylsulfonyl)phenyl)pyrimidine-2,4- diamine; |
| 27 | 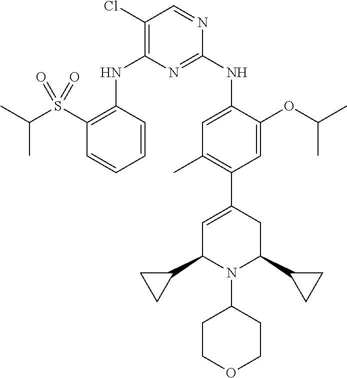 [SMILES: Cc1cc(Nc2ncc(Cl)c(Nc3ccccc3S(=O)(=O)C(C)C)n2)c(OC(C)C)cc1C1=C[C@H](C2CC2)N(C2CCOCC2)[C@H](C2CC2)C1] | 5-chloro-N²-(4-((cis)-2,6-dicyclopropyl-1- (tetrahydro-2H-pyran-4-yl)-1,2,3,6- tetrahydropyridin-4-yl)-2-isopropoxy-5- methylphenyl)-N⁴-(2- (isopropylsulfonyl)phenyl)pyrimidine-2,4- diamine; |
| 28 | 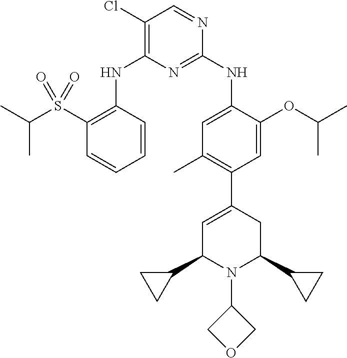 [SMILES: Cc1cc(Nc2ncc(Cl)c(Nc3ccccc3S(=O)(=O)C(C)C)n2)c(OC(C)C)cc1C1=C[C@H](C2CC2)N(C2COC2)[C@H](C2CC2)C1] | 5-chloro-N²-(4-((cis)-2,6-dicyclopropyl-1-(oxetan- 3-yl)-1,2,3,6-tetrahydropyridin-4-yl)-2- isopropoxy-5-methylphenyl)-N⁴-(2- (isopropylsulfonyl)phenyl)pyrimidine-2,4- diamine; |
| 29 | 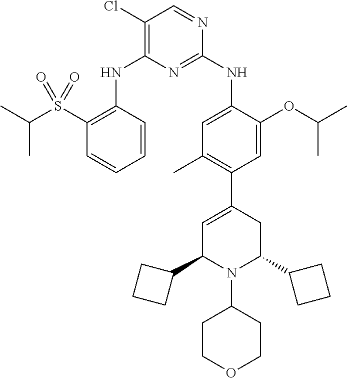 [SMILES: Cc1cc(Nc2ncc(Cl)c(Nc3ccccc3S(=O)(=O)C(C)C)n2)c(OC(C)C)cc1C1=C[C@H](C2CCC2)N(C2CCOCC2)[C@@H](C2CCC2)C1] | 5-chloro-N²-(4-((trans)-2,6-dicyclobutyl-1- (tetrahydro-2H-pyran-4-yl)-1,2,3,6- tetrahydropyridin-4-yl)-2-isopropoxy-5- methylphenyl)-N⁴-(2- (isopropylsulfonyl)phenyl)pyrimidine-2,4- diamine; |
| 30 | 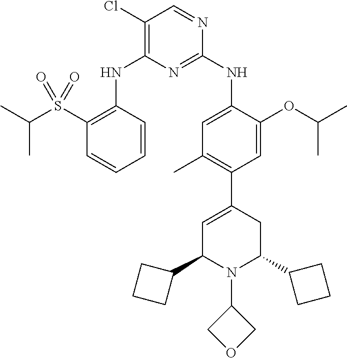 [SMILES: Cc1cc(Nc2ncc(Cl)c(Nc3ccccc3S(=O)(=O)C(C)C)n2)c(OC(C)C)cc1C1=C[C@H](C2CCC2)N(C2COC2)[C@@H](C2CCC2)C1] | 5-chloro-N²-(4-((trans)-2,6-dicyclobutyl-1- (oxetan-3-yl)-1,2,3,6-tetrahydropyridin-4-yl)-2- isopropoxy-5-methylphenyl)-N⁴-(2- (isopropylsulfonyl)phenyl)pyrimidine-2,4- diamine; |
| 31 | 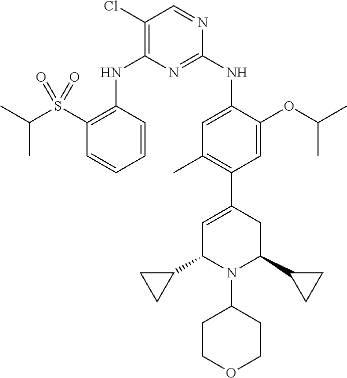 [SMILES: Cc1cc(Nc2ncc(Cl)c(Nc3ccccc3S(=O)(=O)C(C)C)n2)c(OC(C)C)cc1C1=C[C@@H](C2CC2)N(C2CCOCC2)[C@H](C2CC2)C1] | 5-chloro-N²-(4-((trans)-2,6-dicyclopropyl-1- (tetrahydro-2H-pyran-4-yl)-1,2,3,6- tetrahydropyridin-4-yl)-2-isopropoxy-5- methylphenyl)-N⁴-(2- (isopropylsulfonyl)phenyl)pyrimidine-2,4- diamine; or |
| 32 | 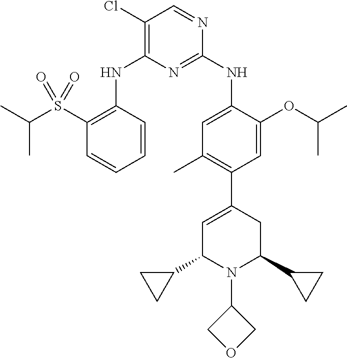 [SMILES: Cc1cc(Nc2ncc(Cl)c(Nc3ccccc3S(=O)(=O)C(C)C)n2)c(OC(C)C)cc1C1=C[C@@H](C2CC2)N(C2COC2)[C@H](C2CC2)C1] | 5-chloro-N²-(4-((trans)-2,6-dicyclopropyl-1- (oxetan-3-yl)-1,2,3,6-tetrahydropyridin-4-yl)-2- isopropoxy-5-methylphenyl)-N⁴-(2- (isopropylsulfonyl)phenyl)pyrimidine-2,4-diamine |

9. The pharmaceutical composition according to claim 1, wherein the ALK inhibitor is 5-chloro-N²-(2-isopropoxy-5-methyl-4-(1-(tetrahydro-2H-pyran-4-yl)-1,2,3,6-tetrahydropyridin-4-yl)phenyl)-N⁴-(2-(isopropylsulfonyl)phenyl)pyrimidine-2,4-diamine, or a pharmaceutically acceptable salt or hydrate thereof.

10. The pharmaceutical composition according to claim 1, wherein the CDK4/6 inhibitor is compound of the formulae (VII) or (VIII), or pharmaceutically acceptable salt thereof: 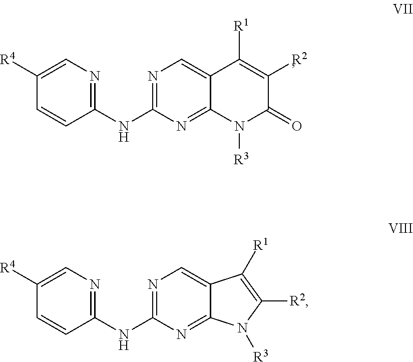
wherein in formulae (VII) or (VIII),
R¹ is selected from the group consisting of hydrogen, C₁₋₄ alkyl, and C₃₋₆ cycloalkyl;
R² is selected from the group consisting of hydrogen, C₁₋₄ alkyl, COR⁵, and CONR⁵R⁶;
R³ is C₃-C₆ cycloalkyl;
R⁴ is 4- to 6-membered heterocyclyl;
R⁵ and R⁶ are independently selected from hydrogen and C₁-C₄ alkyl.

11. (canceled)

12. The pharmaceutical composition according to claim 1, wherein the Mek inhibitor is compound of the formulae (IX) or (X), or pharmaceutically acceptable salt thereof: 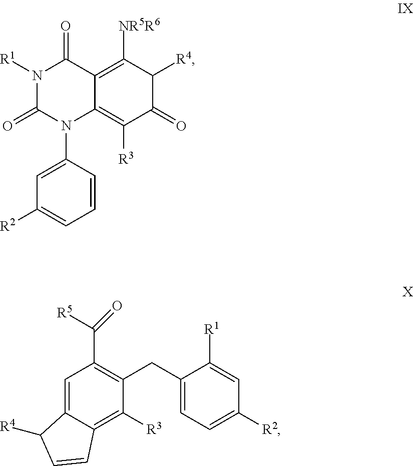
wherein in formulae (IX),
R¹ is selected from the group consisting of hydrogen, C₁-C₄ alkyl, and C₃-C₆ cycloalkyl;
R² is NHC(O)R⁷;
R³, R⁴, R⁵ and R⁷ are independently selected from the group consisting of hydrogen and C₁-C₄ alkyl;
R⁶ is selected from the group consisting of hydrogen, C₆-C₁₀ aryl group, wherein the aryl group is optionally substituted with one or two halogen groups;
wherein in formulae (X),
R¹ is selected from the group consisting of C₁-C₄ alkyl and halogen;
R² is selected from the group consisting of —OCF₃ and halogen;
R³ is selected from the group consisting of hydrogen and halogen;
R⁴ is selected from the group consisting of C₁-C₄ alkyl, and C₃-C₆ cycloalkyl;
R⁵ is NR⁶OR⁷, wherein R⁶ and R⁷ are independently selected from the group consisting of hydrogen and C₁-C₄ alkyl, and when R⁷ is C₁-C₄ alkyl, it is optionally substituted with hydroxy group.

13. (canceled)

14. The pharmaceutical composition according to claim 1, wherein the chemotherapeutic agent comprises platinum or belongs to terpenoid alkaloid.

15. (canceled)

16. The pharmaceutical composition according to claim 1, wherein the MDM2 inhibitor is compound of the formula (XI) or pharmaceutically acceptable salt thereof: 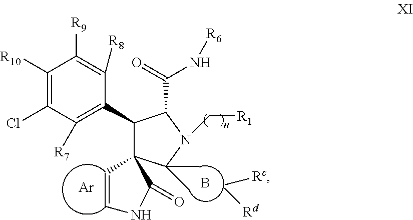
wherein, 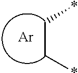
is 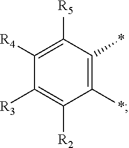
B is 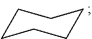 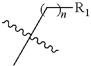
is hydrogen, CH₃, CH₂CH₃, C₃ alkyl or C₄ alkyl;
R₂ is hydrogen; R₃ is halogen; R₄ and R₅ are hydrogen;
R₆ is 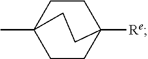
R₇ is halogen; each of R₈, R₉, and R₁₀ is H;
R^{e} is —C(O)OH, —C(O)NH₂, or —C(O)NHSO₂CH₃.

17. (canceled)

18. The pharmaceutical composition according to claim 1, wherein the HDAC inhibitor is compound of the formula (XII) or pharmaceutically acceptable salt thereof: 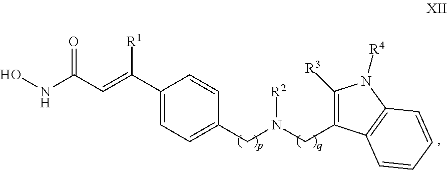
wherein,
R¹ and R⁴ are independently selected from the group consisting of hydrogen and C₁-C₄ alkyl;
R² is selected from the group consisting of hydrogen, —CH₂OH, —CH₂CH₂OH, and —CH₂CH₂CH₂OH;
R³ is selected from the group consisting of hydrogen, C₁-C₄ alkyl, and C₃-C₆ cycloalkyl;
p equals 1-3, and q equals 1-3.

19. (canceled)

20. The pharmaceutical composition according to claim 1, wherein the PD-1 inhibitor is anti-PD-1 antibody.

21. The pharmaceutical composition according to claim 1, wherein the PARP inhibitor is compound of the formula (XIII) or pharmaceutically acceptable salt thereof: 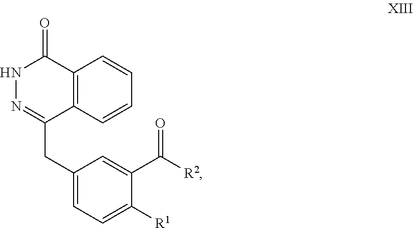
wherein,
R¹ is selected from the group consisting of hydrogen, C₁-C₄ alkyl, and halogen;
R² is 4- to 8-membered heterocyclyl, which is optionally substituted with —C(O)R³, wherein
R³ is C₃-C₆ cycloalkyl.

22. (canceled)

23. The pharmaceutical composition according to claim 1, wherein the VEGF inhibitor is compound of the formula (XIV) or pharmaceutically acceptable salt thereof: 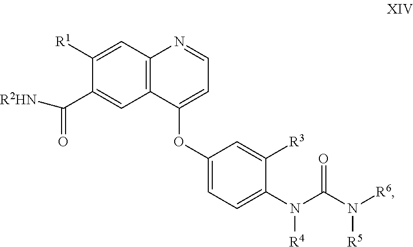
wherein,
R¹ is selected from the group consisting of C₁-C₄ alkyl, and C₁-C₄ alkyloxy;
R², R⁴ and R⁵ are independently selected from the group consisting of hydrogen, and C₁-C₄ alkyl;
R³ is selected from the group consisting of halogen, and C₁-C₄ alkyl;
R6 is selected from the group consisting of C1-C4 alkyl, and C3-C6 cycloalkyl.

24. (canceled)

25. The pharmaceutical composition according to claim 1, wherein the BCR-ABL inhibitor is compound of the formula (XV) or pharmaceutically acceptable salt thereof: 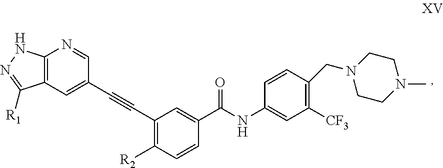
wherein,
R¹ is hydrogen, C₁-C₄ alkyl, C₃-C₆ cycloalkyl, C₁-C₄ alkyloxy, or phenyl; and R2 is hydrogen, C₁-C₄ alkyl, C₃-C₆ cycloalkyl, or halogen.

26-27. (canceled)

28. The pharmaceutical composition according to claim 1, wherein the weight ratio between the ALK inhibitor and the one or more anticancer reagents is 0.005-5000:0.005-5000, for example, 0.05-1500:0.005-5000, 0.1-6:0.005-4, 100:0.5-400, 100:1-350, 100:2-300, 100:5-200, 100:10-150, 100:20-100, 100:30-90, 100:20-80.

29. A method for treating or suppressing a cancer, reducing its severity, lowering its risk or inhibiting its metastasis in an individual, comprising administering to the individual a therapeutically effective amount of an ALK inhibitor, and a therapeutically effective amount of one or more anticancer reagents selected from a CDK4/6 inhibitor, an Mek inhibitor, a BRAF inhibitor, a chemotherapeutic agent, an MDM2 inhibitor, an HDAC inhibitor, a PD-1 inhibitor, a PARP inhibitor, a VEGF inhibitor and a BCR-ABL inhibitor.

30. The method according to claim 29, wherein the ALK inhibitor is administrated in an amount of from about 0.005 mg/day to about 5000 mg/day, such as an amount of about 0.005, 0.05, 0.5, 5, 10, 20, 30, 40, 50, 100, 150, 200, 250, 300, 350, 400, 450, 500, 550, 600, 650, 700, 750, 800, 850, 900, 950, 1000, 1500, 2000, 2500, 3000, 3500, 4000, 4500 or 5000 mg/day.

31. The method according to claim 29, wherein the ALK inhibitor is administrated in an amount of from about 1 ng/kg to about 200 mg/kg, about 1 μg/kg to about 100 mg/kg, or about 1 mg/kg to about 50 mg/kg per unit dose, for example, administrated in an amount of about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg per unit dose, and administrated with one or more (e.g., 1, 2, 3, 4, 5, 6, 7, 8, 9 or 10) unit doses per day.

32-34. (canceled)

35. The method according to claim 29, wherein the ALK inhibitor, and the one or more anticancer reagents are administered continuously for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days, or at least 50 days.

36-42. (canceled)

43. A kit, comprising:
a first component in a first container, the first component comprising an ALK inhibitor (as defined in claim 2), and optionally a pharmaceutically acceptable carrier;
a second component in a second container, the second component comprising an anticancer reagent selected from a CDK4/6 inhibitor, an Mek inhibitor, a BRAF inhibitor, a chemotherapeutic agent, an MDM2 inhibitor, an HDAC inhibitor, a PD-1 inhibitor, a PARP inhibitor, a VEGF inhibitor and a BCR-ABL inhibitor, and optionally a pharmaceutically acceptable carrier; and
an optional specification.
